(19) United States
(12) Patent Application Publication
(10) Pub. No.: US 2010/0298324 A1
(43) Pub. Date: Nov. 25, 2010

(54) Prolyl Hydroxylase Inhibitors

(75) Inventors: Dimitar B. Gotchev, Hatboro, PA (US); Jian Jin, West Chester, PA (US); Yonghui Wang, Collegeville, PA (US)

Correspondence Address: GlaxoSmithKline;GLOBAL PATENTS -US, UW2220, P. O. BOX 1539, KING OF PRUSSIA, PA, 19406-0939, US

(73) Assignee: SMITH KLINE BEECHAM CORPORATION, Philadelphia, PA (US)

(21) Appl. No.: 12/808,195

(22) PCT Filed: Dec. 19, 2008
(86) PCT No.: PCT/US2008/087580

§ 371 Date: Jun. 15, 2010

Related U.S. Application Data
(60) Provisional application No. 61/014,867, filed on Dec. 19, 2007.

(51) Int. Cl. A61K 31/517; C07D 401/04; C07D 413/14; A61K 31/5377; A61P 7/06

(52) U.S. Cl. 514/235.2; 544/284; 544/119; 514/266.21

(57) ABSTRACT

The invention described herein relates to certain 4-oxo-2-thioxo-1,2,3,4-tetrahydro-7-quinazolinecarboxamide derivatives of formula (I)

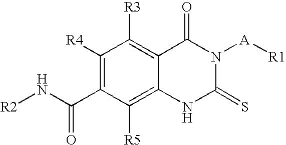

which are antagonists of HIF prolyl hydroxylases and are useful for treating diseases benefiting from the inhibition of this enzyme, anemia being one example.

### FIELD OF THE INVENTION

This invention relates to certain 4-oxo-2-thioxo-1,2,3,4-tetrahydro-7-quinazolinecarboxamide derivatives that are inhibitors of HIF prolyl hydroxylases, and thus have use in treating diseases benefiting from the inhibition of this enzyme, anemia being one example.

### BACKGROUND OF THE INVENTION

Anemia occurs when there is a decrease or abnormality in red blood cells, which leads to reduced oxygen levels in the blood. Anemia occurs often in cancer patients, particularly those receiving chemotherapy. Anemia is often seen in the elderly population, patients with renal disease, and in a wide variety of conditions associated with chronic disease.

Frequently, the cause of anemia is reduced erythropoietin (Epo) production resulting in prevention of erythropoiesis (maturation of red blood cells). Epo production can be increased by inhibition of prolyl hydroxylases that regulate hypoxia inducible factor (HIF).

One strategy to increase erythropoietin (Epo) production is to stabilize and thus increase the transcriptional activity of the HIF. HIF-alpha subunits (HIF-1alpha, HIF-2alpha, and HIF-3alpha) are rapidly degraded by proteosome under normoxic conditions upon hydroxylation of proline residues by prolyl hydroxylases (EGLN1, 2, 3). Proline hydroxylation allows interaction with the von Hippel Lindau (VHL) protein, a component of an E3 ubiquitin ligase. This leads to ubiquitination of HIF-alpha and subsequent degradation. Under hypoxic conditions, the inhibitory activity of the prolyl hydroxylases is suppressed, HIF-alpha subunits are therefore stabilized, and HIF-responsive genes, including Epo, are transcribed. Thus, inhibition of prolyl hydroxylases results in increased levels of HIF-alpha and thus increased Epo production.

The compounds of this invention provide a means for inhibiting these hydroxylases, increasing Epo production, and thereby treating anemia. Ischemia, stroke, and cytoprotection may also be treated by administering these compounds.

### SUMMARY OF THE INVENTION

In the first instance, this invention relates to a compound of formula (I):

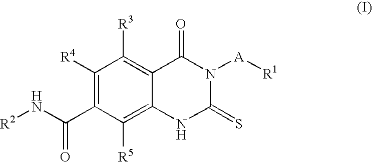

wherein:

R¹is an unsubstituted or substituted 4 to 8-membered mono-cyclic heteroaryl or a 9-11-membered bicyclic heteroaryl ring or an unsubstituted or substituted 4 to 8-membered heterocycloalkyl ring, each containing one or more hetero atoms selected from the group consisting of N, O and S; wherein a carbon atom in said rings can be substituted by one or more groups selected from the group consisting of C₁-C₆alkyl, halogen, —OR⁶, —CN, —C(O)R⁶, and —C(O)OR⁶;

A is a bond, CR⁷R⁸, or NR⁶;

R²is aryl, C₁-C₁₀alkyl-aryl, heteroaryl, C₁-C₁₀alkyl-heteroaryl, C₃-C₈-heterocyclyl, C₁-C₁₀alkyl-C₃-C₈-heterocyclyl, C₁-C₁₀alkyl, C₂-C₁₀alkenyl, C₂-C₁₀alkynyl, C₃-C₈cycloalkyl, C₃-C₈heterocycloalkyl, C₅-C₈cycloalkenyl, (C₂-C₁₀)alkyl-R⁹;

R³, R⁴and R⁵each independently selected from the group consisting of hydrogen, nitro, cyano, halogen, CF₃, —C(O)R⁶, —C(O)OR⁶, —OR⁶, —SR⁶, —S(O)R⁶, —S(O)₂R⁶, —NR⁷R⁸, —CONR⁷R⁸, —N(R⁷)C(O)R⁶, —N(R⁷)C(O)OR⁶, —OC(O)NR⁷R⁸, —N(R⁷)C(O)N⁷R⁸, —P(O)(OR⁶)₂, —SO₂NR⁷R⁸, —N(R⁷)SO₂R⁶, C₁-C₁₀alkyl, C₂-C₁₀alkenyl, C₂-C₁₀alkynyl, C₃-C₈cycloalkyl, C₃-C₈heterocycloalkyl, C₅-C₈cycloalkenyl, aryl, and heteroaryl;

each R⁶is independently selected from the group consisting of hydrogen, C₁-C₁₀alkyl, C₃-C₁₀cycloalkyl, C₃-C₁₀heterocycloalkyl, aryl, and heteroaryl;

R⁷and R⁸are each independently selected from the group consisting of hydrogen, C₁-C₁₀alkyl, C₃-C₁₀cycloalkyl, C₃-C₁₀heterocycloalkyl, aryl, and heteroaryl; or R⁷and R⁸taken together with the nitrogen to which they are attached form a 5- or 6- or 7-membered saturated ring optionally containing one other heteroatom which is oxygen, nitrogen or sulphur;

R⁹is selected from the group consisting of nitro, cyano, halogen, —C(O)R⁶, —C(O)OR⁶, —OR⁶—SR⁶, —S(O)R⁶—NR⁷R⁸, —CONR⁷R⁸, —N(R⁷)C(O)R⁶, —N(R⁷)C(O)OR⁶, —OC(O)NR⁷R⁸, —N(R⁷)C(O)N⁷R⁸, —P(O)(OR⁶)₂, —SO₂NR⁷R⁸, —N(R⁷)SO₂R⁸;

any carbon or heteroatom of R¹, R², R³, R⁴, R⁵, R⁶, R⁷, R⁸, R⁹is unsubstituted or is substituted with one or more substituents independently selected from the group consisting of C₁-C₆alkyl, aryl, heteroaryl, halogen, —OR⁶, —NR⁷R⁸, cyano, nitro, —C(O)R⁶, —C(O)OR⁶, —SR⁶, —S(O)R⁶, —S(O)₂R⁶, —CONR⁷R⁸, —N(R⁷)C(O)R⁶, —N(R⁷)C(O)OR⁶, —OC(O)NR⁷R⁸, —N(R⁷)C(O)NR⁷R⁸, —SO₂NR⁷R⁸, —N(R⁷)SO₂R⁶, C₂-C₁₀alkenyl, C₂-C₁₀alkynyl, C₃-C₈cycloalkyl, C₃-C₈heterocycloalkyl, C₅-C₈cycloalkenyl, aryl or heteroaryl, wherein R⁶, R⁷, and R⁸are the same as defined above;

or a pharmaceutically acceptable salt or solvate thereof.

In a second aspect of the present invention, there is provided a compound of formula (I) or a salt or solvate thereof for use in mammalian therapy, e.g. treating amenia. An example of this therapeutic approach is that of a method for treating anemia caused by increasing the production of erythropoietin (Epo) by inhibiting HIF prolyl hydroxylases comprising administering a compound of formula (I) to a patient in need thereof, neat or admixed with a pharmaceutically acceptable excipient, in an amount sufficient to increase production of Epo.

In a third aspect of the present invention, there is provided a pharmaceutical composition comprising a compound of formula (I) or a salt, solvate, or the like thereof, and one or more of pharmaceutically acceptable carriers, diluents and excipients.

In a fourth aspect, there is provided the use of a compound of formula (I) or a salt or solvate thereof in the preparation of a medicament for use in the treatment of a disorder mediated by inhibiting HIF prolyl hydroxylases, such as an anemia, that can be treated by inhibiting HIF prolyl hydroxylases.

### DETAILED DESCRIPTION OF THE INVENTION

For the avoidance of doubt, unless otherwise indicated, the term “substituted” means substituted by one or more defined groups. In the case where groups may be selected from a number of alternative groups, the selected groups may be the same or different.

The term “independently” means that where more than one substituent is selected from a number of possible substituents, those substituents may be the same or different.

An “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein the term “alkyl” refers to a straight- or branched-chain hydrocarbon radical having the specified number of carbon atoms, so for example, as used herein, the terms “C₁-C₁₀alkyl” refers to an alkyl group having at least 1 and up to 10 carbon atoms respectively. Examples of such branched or straight-chained alkyl groups useful in the present invention include, but are not limited to, methyl, ethyl, n-propyl, isopropyl, isobutyl, n-butyl, t-butyl, n-pentyl, isopentyl, n-hexyl, n-heptyl, n-octyl, n-nonyl, and n-decyl, and branched analogs of the latter 5 normal alkanes.

When the term “alkenyl” (or “alkenylene”) is used it refers to straight or branched hydrocarbon chains containing the specified number of carbon atoms and at least 1 and up to 5 carbon-carbon double bonds. Examples include ethenyl (or ethenylene) and propenyl (or propenylene).

When the term “alkynyl” (or “alkynylene”) is used it refers to straight or branched hydrocarbon chains containing the specified number of carbon atoms and at least 1 and up to 5 carbon-carbon triple bonds. Examples include ethynyl (or ethynylene) and propynyl (or propynylene).

When “cycloalkyl” is used it refers to a non-aromatic, saturated, cyclic hydrocarbon ring containing the specified number of carbon atoms. So, for example, the term “C₃-C₈cycloalkyl” refers to a non-aromatic cyclic hydrocarbon ring having from three to eight carbon atoms. Exemplary “C₃-C₈cycloalkyl” groups useful in the present invention include, but are not limited to, cyclopropyl, cyclobutyl, cyclopentyl, cyclohexyl, cycloheptyl and cyclooctyl.

The term “C₅-C₈cycloalkenyl” refers to a non-aromatic monocyclic carboxycyclic ring having the specified number of carbon atoms and up to 3 carbon-carbon double bonds. “Cycloalkenyl” includes by way of example cyclopentenyl and cyclohexenyl.

Where “C₃-C₈heterocycloalkyl” is used, it means a non-aromatic heterocyclic ring containing the specified number of ring atoms being, saturated or having one or more degrees of unsaturation and containing one or more heteroatom substitutions selected from O, S and/or N. Such a ring may be optionally fused to one or more other “heterocyclic” ring(s) or cycloalkyl ring(s). Examples of “heterocyclic” moieties include, but are not limited to, aziridine, thiirane, oxirane, azetidine, oxetane, thietane, tetrahydrofuran, pyran, 1,4-dioxane, 1,4-dithiane, 1,3-dioxane, 1,3-dioxolane, piperidine, piperazine, 2,4-piperazinedione, pyrrolidine, 2-imidazoline, imidazolidine, pyrazolidine, pyrazoline, morpholine, thiomorpholine, tetrahydrothiopyran, tetrahydrothiophene, and the like.

“Aryl” refers to optionally substituted monocyclic and polycarbocyclic unfused or fused groups having 6 to 14 carbon atoms and having at least one aromatic ring that complies with Hückel's Rule. Examples of aryl groups are phenyl, biphenyl, naphthyl, anthracenyl, phenanthrenyl and the like.

“Heteroaryl” means an optionally substituted aromatic monocyclic ring or polycarbocyclic fused ring system wherein at least one ring complies with Hückel's Rule, has the specified number of ring atoms, and that ring contains at least one heteratom selected from N, O, and/or S. Examples of “heteroaryl” groups include furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridinyl, pyridazinyl, pyrazinyl, pyrimidinyl, quinolinyl, isoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, benzofuranyl, benzothiophenyl, benzimidazolyl, benzthiazolyl, indolizinyl, indolyl, isoindolyl, and indazolyl.

The substituents on aryl or heteroaryl can be selected from the group consisting of hydrogen, nitro, cyano, halogen, CF₃, —C(O)R⁶, —C(O)OR⁶, —OR⁶, —SR⁶, —S(O)R⁶, —S(O)₂R⁶, —NR⁷R⁸, —CONR⁷R⁸, —N(R⁷)C(O)R⁶, —N(R⁷)C(O)OR⁶, —OC(O)NR⁷R⁸, —N(R⁷)C(O)N⁷R⁸, —P(O)(OR⁶)₂, —SO₂NR⁷R⁸, —N(R⁷)SO₂R⁶, C₁-C₁₀alkyl, C₂-C₁₀alkenyl, C₂-C₁₀alkynyl, C₃-C₈cycloalkyl, C₃-C₈heterocycloalkyl, C₅-C₈cycloalkenyl, aryl, and heteroaryl;

The term “optionally” means that the subsequently described event(s) may or may not occur, and includes both event(s), which occur, and events that do not occur.

The term “solvate” refers to a complex of variable stoichiometry formed by a solute and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

Herein, the term “pharmaceutically-acceptable salts” refers to salts that retain the desired biological activity of the subject compound and exhibit minimal undesired toxicological effects. These pharmaceutically-acceptable salts may be prepared in situ during the final isolation and purification of the compound, or by separately reacting the purified compound in its free acid or free base form with a suitable base or acid, respectively.

In certain embodiments, compounds according to Formula I may contain an acidic functional group, one acidic enough to form salts. Representative salts include pharmaceutically-acceptable metal salts such as sodium, potassium, lithium, calcium, magnesium, aluminum, and zinc salts; carbonates and bicarbonates of a pharmaceutically-acceptable metal cation such as sodium, potassium, lithium, calcium, magnesium, aluminum, and zinc; pharmaceutically-acceptable organic primary, secondary, and tertiary amines including aliphatic amines, aromatic amines, aliphatic diamines, and hydroxy alkylamines such as methylamine, ethylamine, 2-hydroxyethylamine, diethylamine, triethylamine, ethylenediamine, ethanolamine, diethanolamine, and cyclohexylamine.

In certain embodiments, compounds according to Formula (I) may contain a basic functional group and are therefore capable of forming pharmaceutically-acceptable acid addition salts by treatment with a suitable acid. Suitable acids include pharmaceutically-acceptable inorganic acids and pharmaceutically-acceptable organic acids. Representative pharmaceutically-acceptable acid addition salts include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), and napthalene-2-sulfonate.

Compounds of particular interest include those wherein:

R¹is an unsubstituted or substituted 4 to 8-membered mono-cyclic heteroaryl, a 9-11-membered bicyclic heteroaryl ring or an unsubstituted or substituted 4 to 8-membered heterocycloalkyl ring; each containing one or more hetero atoms selected from the group consisting of N, O and S; wherein a carbon atom in any of said rings can be substituted by one or more groups selected from the group consisting of C₁-C₆alkyl, halogen, —OR⁶, —CN, —C(O)R⁶, and —C(O)OR⁶;

A is a bond, CH₂, or NH;

R²is aryl, C₁-C₁₀alkyl-aryl, heteroaryl, C₁-C₁₀alkyl-heteroaryl, C₁-C₁₀alkyl heterocyclyl, C₁-C₁₀alkyl-heterocyclyl, hydrocarbyl, (C₂-C₁₀)alkyl-R⁹;

R³, R⁴and R⁵each independently selected from the group consisting of hydrogen, nitro, cyano, halogen, CF₃, —C(O)R⁶, —C(O)OR⁶, —OR⁶, —SR⁶, —S(O)R⁶, —S(O)₂R⁶, —NR⁷R⁸, —CONR⁷R⁸, —N(R⁷)C(O)R⁶, —N(R⁷)C(O)OR⁶, —OC(O)NR⁷R⁸, —N(R⁷)C(O)N⁷R⁸, —P(O)(OR⁶)₂, —SO₂NR⁷R⁸, —N(R⁷)SO₂R⁶, C₁-C₁₀alkyl, C₂-C₁₀alkenyl, C₂-C₁₀alkynyl, C₃-C₈cycloalkyl, C₃-C₈heterocycloalkyl, C₅-C₈cycloalkenyl, aryl, and heteroaryl;

each R⁶is independently selected from the group consisting of hydrogen, C₁-C₁₀alkyl, C₃-C₁₀cycloalkyl, C₃-C₁₀heterocycloalkyl, aryl, and heteroaryl;

R⁷and R⁸are each independently selected from the group consisting of hydrogen, C₁-C₁₀alkyl, C₃-C₁₀cycloalkyl, C₃-C₁₀heterocycloalkyl, aryl, and heteroaryl; or R⁷and R⁸taken together with the nitrogen to which they are attached form a 5- or 6- or 7-membered saturated ring optionally containing one other heteroatom which is oxygen, nitrogen or sulphur;

R⁹is selected from the group consisting of nitro, cyano, halogen, —C(O)R⁶, —C(O)OR⁶, —OR⁶—SR⁶, —S(O)R⁶—NR⁷R⁸, —CONR⁷R⁸, —N(R⁷)C(O)R⁶, —N(R⁷)C(O)OR⁶, —OC(O)NR⁷R⁸, —N(R⁷)C(O)N⁷R⁸, —P(O)(OR⁶)₂, —SO₂NR⁷R⁸, —N(R⁷)SO₂R⁸;

any carbon or heteroatom of R², R³, R⁴, R⁵, R⁶, R⁷, R⁸, R⁹, is unsubstituted or where possible substituted with one or more substituents independently selected from the group consisting of C₁-C₆alkyl, aryl, heteroaryl, halogen, —OR⁶, —NR⁷R⁸, cyano, nitro, —C(O)R⁶, —C(O)OR⁶, —SR⁶, —S(O)R⁶, —S(O)₂R⁶, —CONR⁷R⁸, —N(R⁷)C(O)R⁶, —N(R⁷)C(O)OR⁶, —OC(O)NR⁷R⁸, —N(R⁷)C(O)NR⁷R⁸, —SO₂NR⁷R⁸, —N(R⁷)SO₂R⁶, C₂-C₁₀alkenyl, C₂-C₁₀alkynyl, C₃-C₈cycloalkyl, C₃-C₈heterocycloalkyl, C₅-C₈cycloalkenyl, aryl or heteroaryl, wherein R⁶, R⁷, and R⁸are the same as defined above;

or a pharmaceutically acceptable salt or solvate thereof.

Compounds of further interest are those wherein:

R¹is an unsubstituted or substituted 4 to 8-membered mono-cyclic heteroaryl or a 9-11-membered bicyclic heteroaryl ring containing one or more hetero atoms selected from the group consisting of N, O and S; wherein a carbon atom in said rings can be substituted by one or more groups selected from the group consisting of C₁-C₆alkyl, halogen, —OR⁶, —CN, —C(O)R⁶, and —C(O)OR⁶;

A is a bond, or CH₂;

R²is aryl, C₁-C₁₀alkyl-aryl, heteroaryl, C₁-C₁₀alkyl-heteroaryl, C₅-C₈cycloalkyl-heterocyclyl, (C₂-C₁₀)alkyl-R⁹;

R³, R⁴and R⁵each independently selected from the group consisting of hydrogen, nitro, cyano, halogen, CF₃, —C(O)R⁶, —C(O)OR⁶, —OR⁶, —SR⁶, —S(O)R⁶, —S(O)₂R⁶, —NR⁷R⁸, —CONR⁷R⁸, —N(R⁷)C(O)R⁶, —N(R⁷)C(O)OR⁶, —OC(O)NR⁷R⁸, —N(R⁷)C(O)N⁷R⁸, —P(O)(OR⁶)₂, —SO₂NR⁷R⁸, —N(R⁷)SO₂R⁶, C₁-C₁₀alkyl, C₂-C₁₀alkenyl, C₂-C₁₀alkynyl, C₃-C₈cycloalkyl, C₃-C₈heterocycloalkyl, C₅-C₈cycloalkenyl, aryl, and heteroaryl;

each R⁶is independently selected from the group consisting of hydrogen, C₁-C₁₀alkyl, C₃-C₁₀cycloalkyl, C₃-C₁₀heterocycloalkyl, aryl, and heteroaryl;

R⁷and R⁸are each independently selected from the group consisting of hydrogen, C₁-C₁₀alkyl, C₃-C₁₀cycloalkyl, C₃-C₁₀heterocycloalkyl, aryl, and heteroaryl; or R⁷and R⁸taken together with the nitrogen to which they are attached form a 5- or 6- or 7-membered saturated ring optionally containing one other heteroatom which is oxygen, nitrogen or sulphur;

R⁹is selected from the group consisting of nitro, cyano, halogen, —C(O)R⁶, —C(O)OR⁶, —OR⁶—SR⁶, —S(O)R⁶—NR⁷R⁸, —CONR⁷R⁸, —N(R⁷)C(O)R⁶, —N(R⁷)C(O)OR⁶, —OC(O)NR⁷R⁸, —N(R⁷)C(O)N⁷R⁸, —P(O)(OR⁶)₂, —SO₂NR⁷R⁸, —N(R⁷)SO₂R⁸;

any carbon or heteroatom of R¹, R², R³, R⁴, R⁵, R⁶, R⁷, R⁸, R⁹is unsubstituted or, where possible, is substituted with one or more substituents independently selected from C₁-C₆alkyl, aryl, heteroaryl, halogen, —OR⁶, —NR⁷R⁸, cyano, nitro, —C(O)R⁶, —C(O)OR⁶, —SR⁶, —S(O)R⁶, —S(O)₂R⁶, —CONR⁷R⁸, —N(R⁷)C(O)R⁶, —N(R⁷)C(O)OR⁶, —OC(O)NR⁷R⁸, —N(R⁷)C(O)NR⁷R⁸, —SO₂NR⁷R⁸, —N(R⁷)SO₂R⁶, C₂-C₁₀alkenyl, C₂-C₁₀alkynyl, C₃-C₈cycloalkyl, C₃-C₈heterocycloalkyl, C₅-C₈cycloalkenyl, aryl or heteroaryl, wherein R⁶, R⁷, and R⁸are the same as defined above;

or a pharmaceutically acceptable salt or solvate thereof.

Compounds of further interest are those wherein:

R¹is an unsubstituted or substituted 4 to 8-membered mono-cyclic heteroaryl or a 9-11-membered bicyclic heteroaryl ring containing one or more hetero atoms selected from the group consisting of N, O and S; wherein a carbon atom in said rings can be substituted by one or more groups selected from the group consisting of C₁-C₆alkyl, halogen, —OR⁶, —CN, —C(O)R⁶, and —C(O)OR⁶;

A is a bond or CH₂

R²is aryl, C₁-C₁₀alkyl-aryl;

R³, R⁴and R⁵each independently selected from the group consisting of hydrogen, nitro, cyano, halogen, CF₃, —C(O)R⁶, —C(O)OR⁶, —OR⁶, —SR⁶, —S(O)R⁶, —S(O)₂R⁶, —NR⁷R⁸, —CONR⁷R⁸, —N(R⁷)C(O)R⁶, —N(R⁷)C(O)OR⁶, —OC(O)NR⁷R⁸, —N(R⁷)C(O)N⁷R⁸, —P(O)(OR⁶)₂, —SO₂NR⁷R⁸, —N(R⁷)SO₂R⁶, C₁-C₁₀alkyl, C₂-C₁₀alkenyl, C₂-C₁₀alkynyl, C₃-C₈cycloalkyl, C₃-C₈heterocycloalkyl, C₅-C₈cycloalkenyl, aryl, and heteroaryl;

each R⁶is independently selected from the group consisting of hydrogen, C₁-C₁₀alkyl, C₃-C₁₀cycloalkyl, C₃-C₁₀heterocycloalkyl, aryl, and heteroaryl;

R⁷and R⁸are each independently selected from the group consisting of hydrogen, C₁-C₁₀alkyl, C₃-C₁₀cycloalkyl, C₃-C₁₀heterocycloalkyl, aryl, and heteroaryl; or R⁷and R⁸taken together with the nitrogen to which they are attached form a 5- or 6- or 7-membered saturated ring optionally containing one other heteroatom which is oxygen, nitrogen or sulphur;

or a pharmaceutically acceptable salt or solvate thereof.

Specific compounds exemplified herein via the Examples set out below.

Processes for preparing the compound of formula (I) are also within the ambit of this invention (see Illustrated Methods of Preparation section). The compounds of formula (I) may be prepared in crystalline or non-crystalline form, and, if crystalline, may optionally be solvated, e.g. as the hydrate. This invention includes within its scope stoichiometric solvates (e.g. hydrates) as well as compounds containing variable amounts of solvent (e.g. water).

Certain of the compounds described herein may contain one or more chiral atoms, or may otherwise be capable of existing as two enantiomers. The compounds claimed below include mixtures of enantiomers as well as purified enantiomers or enantiomerically enriched mixtures. Also included within the scope of the invention are the individual isomers of the compounds represented by formula (I), or claimed below, as well as any wholly or partially equilibrated mixtures thereof. The present invention also covers the individual isomers of the claimed compounds as mixtures with isomers thereof in which one or more chiral centers are inverted. Also, it is understood that any tautomers and mixtures of tautomers of the claimed compounds are included within the scope of the compounds of formula (I) as disclosed herein above or claimed herein below.

Where there are different isomeric forms they may be separated or resolved one from the other by conventional methods, or any given isomer may be obtained by conventional synthetic methods or by stereospecific or asymmetric syntheses.

While it is possible that, for use in therapy, a compound of formula (I), as well as salts, solvates and the like, may be administered as a neat preparation, i.e. no additional carrier, the more usual practice is to present the active ingredient confected with a carrier or diluent. Accordingly, the invention further provides pharmaceutical compositions, which includes a compound of formula (I) and salts, solvates and the like, and one or more pharmaceutically acceptable carriers, diluents, or excipients. The compounds of formula (I) and salts, solvates, etc, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the formula (I), or salts, solvates etc, with one or more pharmaceutically acceptable carriers, diluents or excipients.

It will be appreciated by those skilled in the art that certain protected derivatives of compounds of formula (I), which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. Further, certain compounds of the invention may act as prodrugs of other compounds of the invention. All protected derivatives and prodrugs of compounds of the invention are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. Preferred prodrugs for compounds of the invention include: esters, carbonate esters, hemi-esters, phosphate esters, nitro esters, sulfate esters, sulfoxides, amides, carbamates, azo-compounds, phosphamides, glycosides, ethers, acetals and ketals.

Pharmaceutical compositions may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the formula (I), depending on the condition being treated, the route of administration and the age, weight and condition of the patient, or pharmaceutical compositions may be presented in unit dose forms containing a predetermined amount of active ingredient per unit dose. Preferred unit dosage compositions are those containing a daily dose or sub-dose, as herein above recited, or an appropriate fraction thereof, of an active ingredient. Furthermore, such pharmaceutical compositions may be prepared by any of the methods well known in the pharmacy art.

Pharmaceutical compositions may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by bringing into association a compound of formal (I) with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of a compound of formula (I). Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit pharmaceutical compositions for oral administration can be microencapsulated. The formulation can also be prepared to prolong or sustain the release as for example by coating or embedding particulate material in polymers, wax or the like.

Pharmaceutical compositions adapted for rectal administration may be presented as suppositories or as enemas.

Pharmaceutical compositions adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The pharmaceutical compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the pharmaceutical compositions may include other agents conventional in the art having regard to the type of formulation in question, for example those suitable for oral administration may include flavouring agents.

A therapeutically effective amount of a compound of the present invention will depend upon a number of factors including, for example, the age and weight of the intended recipient, the precise condition requiring treatment and its severity, the nature of the formulation, and the route of administration, and will ultimately be at the discretion of the attendant prescribing the medication. However, an effective amount of a compound of formula (I) for the treatment of anemia will generally be in the range of 0.1 to 100 mg/kg body weight of recipient per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, etc., may be determined as a proportion of the effective amount of the compound of formula (I) per se. It is envisaged that similar dosages would be appropriate for treatment of the other conditions referred to above.

### Chemical Background:

The compounds of this invention may be made by a variety of methods, including standard chemistry. Any previously defined variable will continue to have the previously defined meaning unless otherwise indicated. Illustrative general synthetic methods are set out below and then specific compounds of the invention as prepared are given in the examples.

Compounds of general formula (I) and (II) may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting. Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula (I) and (II). Those skilled in the art will recognize if a stereocenter exists in compounds of formula (I) and (II). Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be affected by any suitable method known in the art. See, for example, Stereochemistry of Organic Compounds by E. L. Eliel, S. H. Wilen, and L. N. Mander (Wiley-Interscience, 1994).

The compounds described herein may be made from commercially available starting materials or synthesized using known organic, inorganic and/or enzymatic processes.

The following abeviations are used in the following pages:

| | |
|---|---|
| ACN | Acetonitrile |
| DCE | 1,2-dichloroethane |
| DCM | Dichloromethane |
| DIEA | Diisopropylethylamine |
| DMAP | 4-dimethylaminopyridine |
| DMF | N,N-dimethylformamide |
| DMHB | 2,6-dimethoxy-4-polystyrenebenzyloxy-benzaldehyde |
| resin | |
| DMSO | Dimethylsulfoxide |
| EDC | 1-(3-Dimethylaminopropyl)-3-ethylcarbodiiminde HCl |
| HATU | O-(7-azabenzotriazol-1-yl)-N,N,N′,N′-tetramethyluronium |
| | hexafluorophosphate |
| HOAc | Acetic acid |
| NMP | N-methylpyrrolidone |
| PS-DCC | Polymer supported dicyclohexylcarbodiimide |
| Rt or | room temperature |
| rt | |
| Sat. | Saturated |
| TBTU | O-(benzotriazol-1-yl)-N,N,N′,N′-tetramethyluronium |
| | tetrafluoroborate |
| TEA | Triethylamine |
| TFA | Trifluoroacetic acid |
| THF | Tetrahydrofuran |
| YMC | ????? (prep chromatography) |

### Illustrated Methods of Preparation

### Schemes

Included in the present invention is a process according to Schemes 1, 2 and 3 for the synthesis of the compounds:

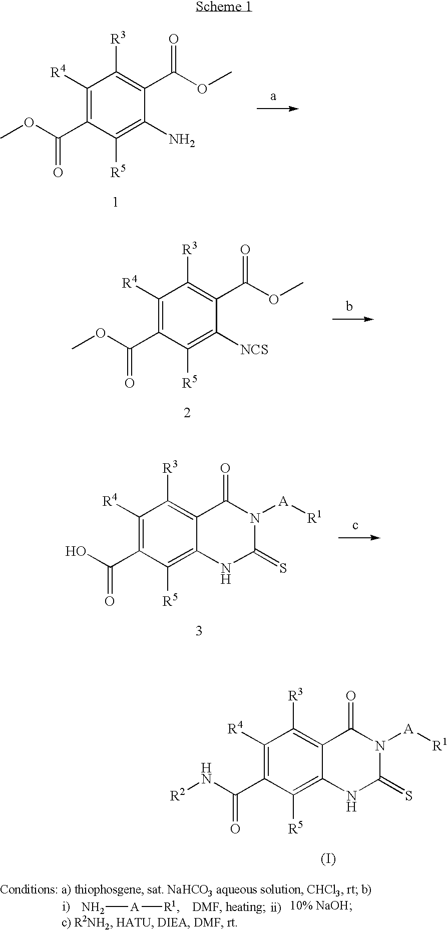

Dimethyl 2-amino-1,4-benzenedicarboxylate or appropriately substituted dimethyl 2-amino-1,4-benzenedicarboxylates 1 react with thiophosgene in sat NaHCO₃and CHCl₃at the room temperature to give isothiocyanates 2. A variety of amines or anilines, such as appropriately substituted aminopyridines, aminopyrimidines and aminothiazoles, are added to isothiocyanates 2 in an appropriate solvent, such as DMF or DMSO, by heating or in room temperature to afford cyclized 4-oxo-2-thioxoquinazolinecarboxylates or uncyclized thioureas or a mixture of both depending on the reactivity of amines or anilines. Upon addition of an appropriate base, such as NaOH, the 4-oxo-2-thioxoquinazolinecarboxylates are hydrolyzed to the corresponding acids 3. Uncyclized thioureas, if any, can be easily cyclized and at the same time, hydrolyzed to the acids 3 in the presence of the same base. In some special cases, NaH is used to facilitate formation of thiourea and DIEA is used to faciliate the cyclization depending on the neuclophilicity of amines or anilines. Amide formation of the acids 3 with a variety of amines or anilines, such as appropriately substituted benzylamines, in the presence of a coupling reagent, such as HATU, EDC or PS-DCC, and base, such as DIEA and DMAP, in an appropriate solvent like DMF or DCM/DMF at rt produces the desired compounds of formula (I).

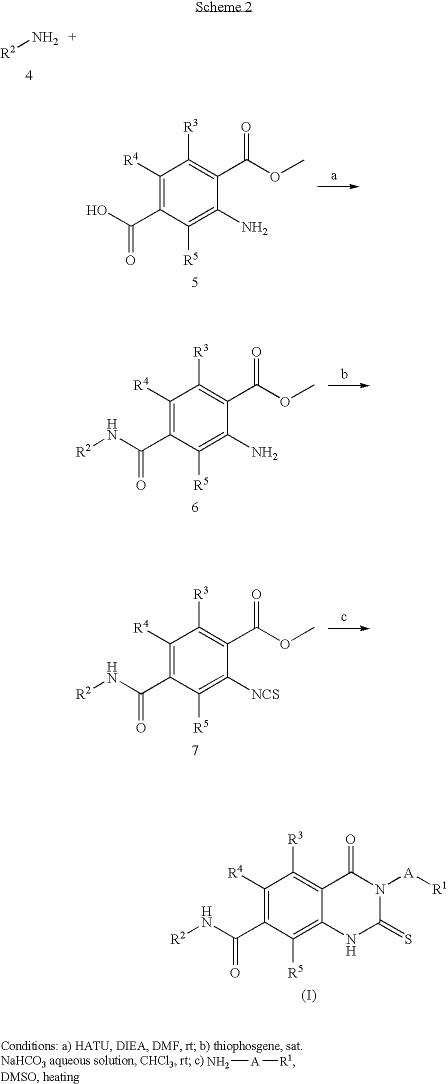

3-Amino-4-[(methyloxy)carbonyl]benzoic acid or appropriately substituted 3-amino-4-[(methyloxy)carbonyl]benzoic acids 5 react with amines 4, such as appropriately substituted benzylamines, in the presence of a coupling reagent, such as HATU, and base, such as DIEA, in an appropriate solvent like DMF at rt to give the amides 6. The amides 6 react with thiophosgene in saturated NaHCO₃and CHCl₃at rt to give isothiocyanates 7. Isothiocyanates 7 react with a variety of amines or anilines, such as appropriately substituted aminopyridines, aminopyrimidines and aminothiazoles, in an appropriate solvent, such as DMF, DMSO or THF by heating or at rt to afford the desired compounds of formula (I). In some cases, when amines used are not reactive enough, an appropriate base is needed to facilitate or speed up the cyclization. The bases used in this application include, but not limit to, NaH, NaOH, and DIEA.

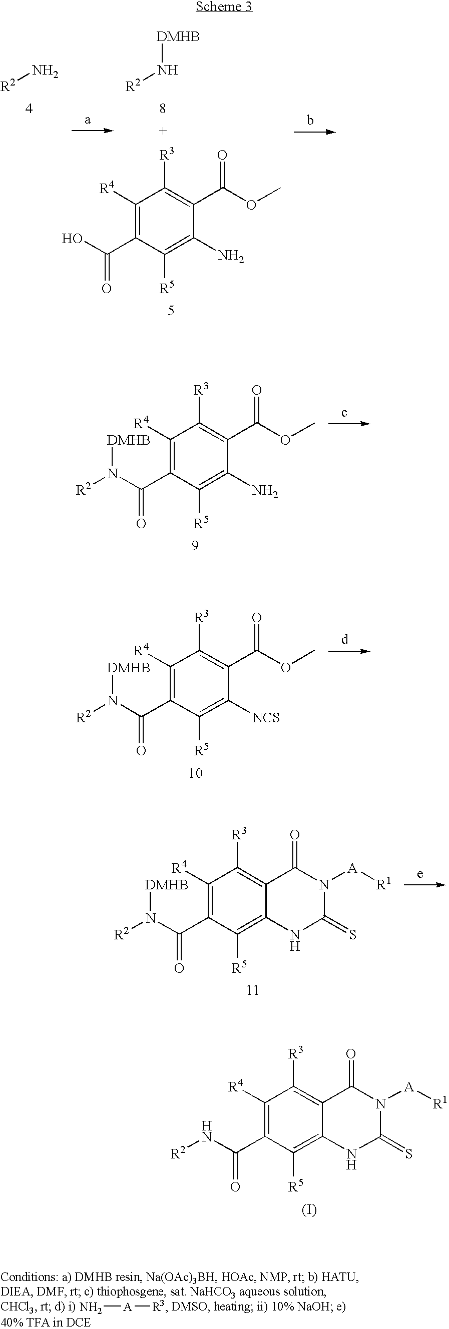

A variety of amines or anilines 4, such as appropriately substituted benzylamines, are loaded to a DMHB resin by reductive amination reaction in NMP using Na(OAc)₃BH as reduction reagent in the presence of HOAc. The DMHB resin-bound amines or anilines 8 react with 3-amino-4-[(methyloxy)carbonyl]benzoic acid or appropriately substituted 3-amino-4-[(methyloxy)carbonyl]benzoic acids 5 in the presence of a coupling reagent, such as HATU, and base, such as DIEA, in an appropriate solvent like DMF in room temperature to give the resin-bound amides 9. The amides 9 react with thiophosgene in saturated NaHCO₃and CHCl₃at rt to give resin-bound isothiocyanates 10. Isothiocyanates 10 react with a variety of amines or anilines, such as appropriately substituted aminopyridines, aminopyrimidines and aminothiazoles, in an appropriate solvent, such as DMF, DMSO, THF by heating or at rt to give the resin-bound products 11, which upon resin cleavage using TFA in DCE, afford the desired compounds of formula (II). In some cases, when amines used are not reactive enough, an appropriate base is needed to facilitate or speed up the cyclization. The bases used in this application include, but not limit to, NaH, NaOH, and DIEA.

### EXAMPLES

The following examples are for illustrative purposes only and are not intended to limit the scope of the invention.

### Example 1

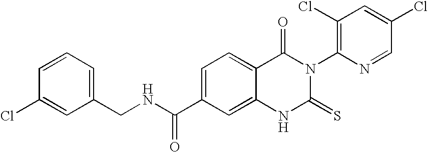

### N-(3-Chlorobenzyl)-3-(3,5-dichloro-2-pyridinyl)-4-oxo-2-thioxo-1,2,3,4-tetrahydro-7-quinazolinecarboxamide

### 1a) Dimethyl 2-isothiocyanato-1,4-benzenedicarboxylate

To a stirred solution of commercially available dimethyl 2-amino-1,4-benzenedicarboxylate (41.84 g, 0.20 mol, 1 eq.) in saturated sodium bicarbonate (500 mL) and chloroform (500 mL) was slowly added thiophosgene (20.5 mL, 0.24 mol, 1.2 eq.) and the mixture was stirred at room temperature for 2.5 hours. Phases were separated and the aqueous was extracted with DCM (3×). The combined organics were dried over anhydrous sodium sulfate, filtered, and concentrated to give dimethyl 2-isothiocyanato-1,4-benzenedicarboxylate as solid (1a, 50.3 g, 100%) which was used for next step without purification. LC/MS: MS+1=252; ¹H-NMR (400 MHz, CDCl₃) δ ppm: 8.01-8.09 (m, 1H), 7.92-8.00 (m, 1H), 4.00 (s, 3H), 3.96 (s, 3H).

### 1b) Methyl 3-(3,5-dichloro-2-pyridinyl)-4-oxo-2-thioxo-1,2,3,4-tetrahydro-7-quinazolinecarboxylate

A mixture of dimethyl 2-isothiocyanato-1,4-benzenedicarboxylate (1a, 1.236 g, 4.9 mmol, 1 eq.) and commercially available 3,5-dichloro-2-aminopyridine (0.88 g, 5.4 mmol, 1.1 eq.) in DMF (10 mL) was heated at 80° C. overnight. The cooled mixture was diluted with water and let stand over the weekend. The resulting solid was collected and purified via preparative HPLC chromatography (YMC 75×30 mm column, 0.1% TFA in water and 0.1% TFA in ACN) to afford 320 mg of methyl 3-(3,5-dichloro-2-pyridinyl)-4-oxo-2-thioxo-1,2,3,4-tetrahydro-7-quinazolinecarboxylate (1b) with 90% purity (yield 17%). LC/MS: M+1=382; ¹H-NMR (400 MHz, DMSO-d₆) δ ppm: 13.61 (s, 1H), 8.74 (d, J=2.27 Hz, 1H), 8.58 (d, J=2.27 Hz, 1H), 8.14 (d, J=8.34 Hz, 1H), 8.06 (d, J=1.26 Hz, 1H), 7.89 (dd, J=8.08, 1.52 Hz, 1H), 3.93 (s, 3H).

### 1c) N-[3-Chlorobenzyl]-3-(3,5-dichloro-2-pyridinyl)-4-oxo-2-thioxo-1,2,3,4-tetrahydro-7-quinazolinecarboxamide

A mixture of methyl 3-(3,5-dichloro-2-pyridinyl)-4-oxo-2-thioxo-1,2,3,4-tetrahydro-7-quinazolinecarboxylate (1b, 224 mg, 0.58 mmol, 1 eq.) and sodium hydroxide (93 mg, 2.32 mmol, 4 eq.) in methanol (10 mL) was heated in the microwave at 140° C. for 5 minutes. Methanol was removed and the remaining mixture was diluted with water and then adjusted to pH 6-7 with 1N hydrochloric acid. Extraction of the product with DCM/methanol (10/1) failed so the aqueous was stripped down then dried in vacuo to give 3-(3,5-dichloro-2-pyridinyl)-4-oxo-2-thioxo-1,2,3,4-tetrahydro-7-quinazolinecarboxylic acid containing sodium chloride. This material was used for next step without further purification. LC/MS: M+1=368.

3-Chlorobenzylamine (45 mg, 0.32 mmol, 1.1 eq.), HATU (122 mg, 0.32 mmol, 1.1 eq.) and DIEA (0.06 mL, 0.32 mmol, 1.1 eq.) were added to a stirred solution of the crude 3-(3,5-dichloro-2-pyridinyl)-4-oxo-2-thioxo-1,2,3,4-tetrahydro-7-quinazolinecarboxylic acid (half amount of the crude product above, as of 0.29 mmol) in dry DMF (5 mL). The mixture was stirred at room temperature for one hour. Solid was filtered off and the filtrate was purified on HPLC (reverse-phase C18 column) under neutral conditions (15-75% gradient in 10 minutes). LC/MS: M+1=491; ¹H-NMR (400 MHz, DMSO-d₆) δ ppm: 13.60 (s, 1H), 9.44 (t, J=5.94 Hz, 1H), 8.74 (d, J=2.02 Hz, 1H), 8.58 (d, J=2.27 Hz, 1H), 8.12 (d, J=8.34 Hz, 1H), 7.93 (d, J=1.26 Hz, 1H), 7.83 (dd, J=8.34, 1H), 7.42-7.30 (m, 4H), 4.515 (d, J=6.06 Hz, 2H).

### Example 2

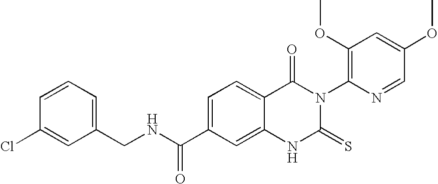

### N-[3-Chlorobenzyl]-3-(3,5-dimethoxy-2-pyridinyl)-4-oxo-2-thioxo-1,2,3,4-tetrahydro-7-quinazolinecarboxamide

### 2a) Methyl 3-(3,5-dimethoxy-2-pyridinyl)-4-oxo-2-thioxo-1,2,3,4-tetrahydro-7-quinazolinecarboxylate

A mixture of dimethyl 2-isothiocyanato-1,4-benzenedicarboxylate (1a, 1.14 g, 4.54 mmol, 1 eq.) and 3,5-dimethoxy-2-aminopyridine (0.77 g, 5.0 mmol, 1.1 eq.) in DMF (10 mL) was heated at 80° C. overnight. The cooled mixture was diluted with water. The resulting solid was collected by filtration and triturated with DCM/hexanes mixture to give 1.36 g (80%) title product (2a) as tan solid. The product was used without further purification. LC/MS: M+1=374; ¹H-NMR (400 MHz, DMSO-d₆) δ ppm: 13.31 (s, 1H), 8.08 (d, J=8.08 Hz, 1H), 8.04 (d, J=1.26 Hz, 1H), 7.83-7.87 (m, 2H), 7.25 (d, J=2.53 Hz, 1H), 3.94 (s, 3H), 3.93 (s, 3H), 3.79 (s, 3H).

### 2b) 3-(3,5-Dimethoxy-2-pyridinyl)-4-oxo-2-thioxo-1,2,3,4-tetrahydro-7-quinazolinecarboxylic acid

A mixture of methyl 3-(3,5-dimethoxy-2-pyridinyl)-4-oxo-2-thioxo-1,2,3,4-tetrahydro-7-quinazolinecarboxylate (2a, 1.1 g, 2.9 mmol, 1 eq.) and sodium hydroxide (464 mg, 11.6 mmol, 4 eq.) in methanol (12 mL) was heated in the microwave at 140° C. for 5 minutes. Methanol was removed and the remaining mixture was diluted with water and then adjusted to pH 6-7 with 1N hydrochloric acid. The resulting fine powder was collected by filtration and washed with water to give 606 mg of the desired product (2b) after drying in the vacuum oven at 30° C. overnight. Part of the product went through the filter so the filtrate was extracted with ethyl acetate to give another 320 mg of the desired product. The combined product was used without further purification. Yield: 926 mg, 89%. LC/MS: M+1=360; ¹H-NMR (400 MHz, DMSO-d₆) δ ppm: 13.70 (br. s, 1H), 13.32 (s, 1H), 8.06 (d, J=8.59 Hz, 1H), 8.03 (s, 1H), 7.86 (d, J=2.53 Hz, 1H), 7.84 (d, J=8.08 Hz, 1H), 7.25 (d, J=2.53 Hz, 1H), 3.94 (s, 3H), 3.79 (s, 3H).

### 2c) N-[3-Chlorobenzyl]-3-(3,5-dimethoxy-2-pyridinyl)-4-oxo-2-thioxo-1,2,3,4-tetrahydro-7-quinazolinecarboxamide

To a stirred solution of 3-(3,5-dimethoxy-2-pyridinyl)-4-oxo-2-thioxo-1,2,3,4-tetrahydro-7-quinazolinecarboxylic acid (2b, 303 mg, 0.84 mmol, 1 eq.) in dry ACN (10 mL) were added 3-chlorobenzylamine (123 mg, 0.85 mmol, 1.01 eq.), DIEA (0.55 mL, 3.16 mmol, 3.7 eq.), and TBTU (313 mg, 0.98 mmol, 1.17 eq.) in order. The mixture was stirred at room temperature for 2 hours (the mixture slowly became cloudy) then diluted with ethyl acetate and washed with saturated sodium bicarbonate (3×). After being dried over anhydrous sodium sulfate, solvents were evaporated. Purification with flash chromatography failed due to the poor solubility of the product. The recovered material was purified on HPLC under acidic condition (20-70% gradient in 10 minutes). Yield: 194 mg, 54%; LC/MS: M+1=483; ¹H-NMR (400 MHz, DMSO-d₆) δ ppm: 13.29 (s, 1H), 9.41 (t, J=5.94 Hz, 1H), 8.06 (d, J=8.34 Hz, 1H), 7.90 (d, J=1.26 Hz, 1H), 7.86 (d, J=2.53 Hz, 1H), 7.79 (dd, J=8.21, 1.39 Hz, 1H), 7.43-7.29 (m, 4H), 7.25 (d, J=2.27 Hz, 1H), 4.51 (d, J=5.81 Hz, 2H), 3.94 (s, 3H), 3.79 (s, 3H).

### Example 3

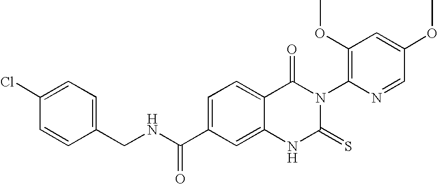

### N-[4-Chlorobenzyl]-3-(3,5-dimethoxy-2-pyridinyl)-4-oxo-2-thioxo-1,2,3,4-tetrahydro-7-quinazolinecarboxamide

To a stirred solution of 3-(3,5-dimethoxy-2-pyridinyl)-4-oxo-2-thioxo-1,2,3,4-tetrahydro-7-quinazolinecarboxylic acid (2b, 303 mg, 0.84 mmol, 1 eq.) in dry ACN (10 mL) were added 4-chlorobenzylamine (132 mg, 0.91 mmol, 1.08 eq.), DIEA (0.55 mL, 3.16 mmol, 3.7 eq.), and TBTU (313 mg, 0.98 mmol, 1.17 eq.) in order. The mixture was stirred at rt for 2 hours (the mixture slowly became cloudy) then diluted with ethyl acetate and washed with saturated sodium bicarbonate (3×). After being dried over anhydrous sodium sulfate, solvents were evaporated. Purification with flash chromatography failed due to the poor solubility of the product. The recovered material was purified on HPLC under acidic condition (20-70% gradient in 10 minutes). Yield: 101 mg, 29%; LC/MS: M+1=483; ¹H-NMR (400 MHz, DMSO-d₆) δ ppm: 13.29 (s, 1H), 9.40 (t, J=5.94 Hz, 1H), 8.05 (d, J=8.34 Hz, 1H), 7.89 (d, J=1.26 Hz, 1H), 7.86 (d, J=2.53 Hz, 1H), 7.78 (dd, J=8.34, 1.26 Hz, 1H), 7.45-7.34 (m, 4H), 7.25 (d, J=2.27 Hz, 1H), 4.49 (d, J=6.06 Hz, 2H), 3.94 (s, 3H), 3.79 (s, 3H).

### Example 4

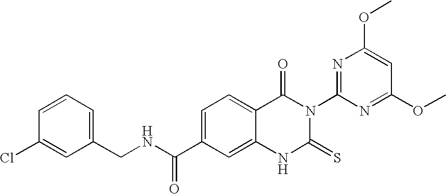

### N-[3-Chlorobenzyl]-3-[4,6-dimethoxy-2-pyrimidinyl]-4-oxo-2-thioxo-1,2,3,4-tetrahydro-7-quinazolinecarboxamide

### 4a) 3-[4,6-Dimethoxy-2-pyrimidinyl]-4-oxo-2-thioxo-1,2,3,4-tetrahydro-7-quinazolinecarboxylic acid

To a solution of dimethyl 2-isothiocyanato-1,4-benzenedicarboxylate (1a, 1.03 g, 4.1 mmol, 1 eq.) in DMF (4 mL) was added 2-amino-4,6-dimethoxypyrimidine (636 mg, 4.1 mmol, 1 eq.) and the mixture was stirred at rt. More DMF (up to 3 mL) was added to give a clear solution within 2 hours. The reaction was stirred for another 2 hours and then heated at 50° C. over the weekend. 10% sodium hydroxide (5 mL) was added and the mixture was stirred at 50° C. for 1 hour. The cooled mixture was acidified with 6N hydrochloric acid to pH 6-7 and the resulting solid was collected by filtration and washed with water. The product (4a) was used without further purification. Yield: 626 mg, 41%; LC/MS: M+1=361; ¹H-NMR (400 MHz, DMSO-d₆) δ ppm: 13.73 (br. s., 1H), 13.44 (s, 1H), 8.09 (d, J=8.59 Hz, 1H), 8.03 (3, 1H), 7.86 (d, J=8.08 Hz, 1H), 6.45 (s, 1H), 3.89 (s, 6H)

### 4b) N-[3-Chlorobenzyl]-3-[4,6-dimethoxy-2-pyrimidinyl]-4-oxo-2-thioxo-1,2,3,4-tetrahydro-7-quinazolinecarboxamide

To a stirred solution of 3-[4,6-dimethoxy-2-pyrimidinyl]-4-oxo-2-thioxo-1,2,3,4-tetrahydro-7-quinazolinecarboxylic acid (4a, 108 mg, 0.3 mmol, 1 eq.) in dry DMF (2 mL) were added 3-chlorobenzylamine (42.5 mg, 0.3 mmol, 1. eq.), DIEA (0.06 mL, 0.33 mmol, 1.1 eq.), and HATU (125 mg, 0.33 mmol, 1.1 eq.) in order. The mixture was stirred at rt for 2 hours and then purified on HPLC under acidic condition (20-75% gradient). Yield: 60 mg, 41%; LC/MS: M+1=483; ¹H-NMR (400 MHz, DMSO-d₆) δ ppm: 13.44 (s, 1H), 9.44 (t, J=5.81 Hz, 1H), 8.08 (d, J=8.59 Hz, 1H), 7.91 (s, 1H), 7.81 (d, J=8.59 Hz, 1H), 7.43-7.18 (m, 4H), 6.45 (s, 1H), 4.51 (d, J=6.06 Hz, 2H), 3.89 (s, 6H)

### Example 5

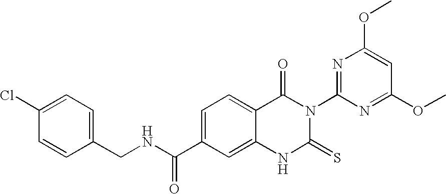

### N-[4-Chlorobenzyl]-3-[4,6-dimethoxy-2-pyrimidinyl]-4-oxo-2-thioxo-1,2,3,4-tetrahydro-7-quinazolinecarboxamide

To a stirred solution of 3-[4,6-dimethoxy-2-pyrimidinyl]-4-oxo-2-thioxo-1,2,3,4-tetrahydro-7-quinazolinecarboxylic acid (4a, 108 mg, 0.3 mmol, 1 eq.) in dry DMF (2 mL) were added 4-chlorobenzylamine (42.5 mg, 0.3 mmol, 1. eq.), DIEA (0.06 mL, 0.33 mmol, 1.1 eq.), and HATU (125 mg, 0.33 mmol, 1.1 eq.) in order. The mixture was stirred at rt for 2 hours and then purified on HPLC under acidic condition (20-75% gradient). Yield: 59 mg, 40%; LC/MS: M+1=483; ¹H-NMR (400 MHz, DMSO-d₆) δ ppm; 13.43 (s, 1H), 9.43 (t, J=6.06 Hz, 1H), 8.08 (d, J=8.08 Hz, 1H), 7.90 (s, 1H), 7.81 (d, J=8.08 Hz, 1H), 7.45-7.34 (m, 4H), 6.45 (s, 1H), 4.49 (d, J=5.56 Hz, 2H), 3.89 (s, 6H)

### Example 6

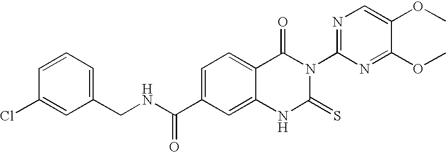

### N-[3-Chlorobenzyl]-3-[5,6-dimethoxy-2-pyrimidinyl]-4-oxo-2-thioxo-1,2,3,4-tetrahydro-7-quinazolinecarboxamide

### 6a) 3-[5,6-Dimethoxy-2-pyrimidinyl]-4-oxo-2-thioxo-1,2,3,4-tetrahydro-7-quinazolinecarboxylic acid

To a solution of dimethyl 2-isothiocyanato-1,4-benzenedicarboxylate (1a, 1.03 g, 4.1 mmol, 1 eq.) in DMF (5 mL) was added 2-amino-5,6-dimethoxypyrimidine (636 mg, 4.1 mmol, 1 eq.) and the mixture was stirred at rt. After 20 minutes, more DMF (2 mL) was added to give a clear solution and the reaction was stirred at rt for another 2 hours and then heated at 50° C. for 10 hours (solid started to precipitate out after 3 hours' heating). 10% sodium hydroxide (5 mL) was added and the mixture was stirred at 50° C. for another hour. The cooled mixture was acidified with 6N hydrochloric acid to pH 6-7 and the resulting solid was collected by filtration and washed with water. The product was used without further purification. Yield: 1.1 g, 75%; LC/MS: M+1=361; ¹H-NMR (400 MHz, DMSO-d₆) δ ppm: 13.71 (br. s., 1H), 13.40 (s, 1H), 8.39 (s 1H), 8.09 (d, J=8.08 Hz, 1H), 8.03 (s, 1H), 7.85 (d, J=9.60 Hz, 1H), 3.97 (s, 3H), 3.91 (s, 3H)

### 6b) N-[3-Chlorobenzyl]-3-[5,6-dimethoxy-2-pyrimidinyl]-4-oxo-2-thioxo-1,2,3,4-tetrahydro-7-quinazolinecarboxamide

To a stirred solution of 3-[5,6-dimethoxy-2-pyrimidinyl]-4-oxo-2-thioxo-1,2,3,4-tetrahydro-7-quinazolinecarboxylic acid (6a, 180 mg, 0.5 mmol, 1 eq.) in dry DMF (3 mL) were added 3-chlorobenzylamine (71 mg, 0.5 mmol, 1. eq.), DIEA (0.1 mL, 0.55 mmol, 1.1 eq.), and HATU (209 mg, 0.55 mmol, 1.1 eq.) in order. The mixture was stirred at rt for 1 hour and then purified on HPLC under acidic condition (15-65% gradient). Yield: 109 mg, 45%; LC/MS: M+1=484; ¹H-NMR (400 MHz, DMSO-d₆) δ ppm: 13.39 (br. s., 1H), 9.42 (t, J=6.06 Hz, 1H), 8.39 (s, 1H), 8.08 (d, J=8.08 Hz, 1H), 7.90 (s, 1H), 7.80 (d, J=8.59 Hz, 1H), 7.44-7.28 (m, 4H), 4.51 (d, J=6.06 Hz, 2H), 3.97 (s, 3H), 3.91 (s, 3H)

### Example 7

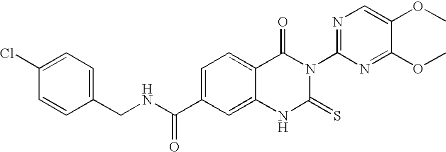

### N-[4-Chlorobenzyl]-3-[5,6-dimethoxy-2-pyrimidinyl]-4-oxo-2-thioxo-1,2,3,4-tetrahydro-7-quinazolinecarboxamide

To a stirred solution of 3-[5,6-dimethoxy-2-pyrimidinyl]-4-oxo-2-thioxo-1,2,3,4-tetrahydro-7-quinazolinecarboxylic acid (6a, 180 mg, 0.5 mmol, 1 eq.) in dry DMF (3 mL) were added 4-chlorobenzylamine (71 mg, 0.5 mmol, 1. eq.), DIEA (0.1 mL, 0.55 mmol, 1.1 eq.), and HATU (209 mg, 0.55 mmol, 1.1 eq.) in order. The mixture was stirred at rt for 1 hour and then purified on HPLC under acidic condition (15-65% gradient). Yield: 141 mg, 58%; LC/MS: M+1=484; ¹H-NMR (400 MHz, DMSO-d₆) δ ppm: 13.39 (br. s., 1H), 9.42 (t, J=6.06 Hz, 1H), 8.39 (s, 1H), 8.07 (d, J=8.08 Hz, 1H), 7.90 (s, 1H), 7.79 (d, J=8.59 Hz, 1H), 7.45-7.34 (m, 4H), 4.49 (d, J=5.56 Hz, 2H), 3.97 (s, 3H), 3.91 (s, 3H)

### Example 8

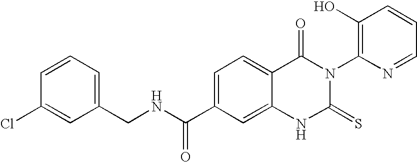

### N-[3-Chlorobenzyl]-3-(3-hydroxy-2-pyridinyl)-4-oxo-2-thioxo-1,2,3,4-tetrahydro-7-quinazolinecarboxamide

### 8a) 3-(3-Hydroxy-2-pyridinyl)-4-oxo-2-thioxo-1,2,3,4-tetrahydro-7-quinazolinecarboxylic acid

To a solution of dimethyl 2-isothiocyanato-1,4-benzenedicarboxylate (1a, 1.03 g, 4.1 mmol, 1 eq.) in DMF (5 mL) was added 2-amino-3-hydroxypyridine (451 mg, 4.1 mmol, 1 eq.) and the mixture was stirred at rt overnight. 10% Sodium hydroxide (5 mL) was added and the mixture was stirred at 50° C. for one hour. The cooled mixture was acidified with 6N hydrochloric acid to pH 6-7 and the crude acid was purified on HPLC under neutral condition (10-50% gradient). Yield: 722 mg, 56%; LC/MS: M+1=316; ¹H-NMR (400 MHz, DMSO-d₆) δ ppm: 13.68 (br. s., 1H), 13.26 (s, 1H), 10.14 (s, 1H), 8.07 (d, J=8.08 Hz, 1H), 8.04-8.00 (m, 2H), 7.84 (d, J=9.60 Hz, 1H), 7.36 (s, 1H), 7.35 (s, 1H)

### 8b) N-[3-Chlorobenzyl]-3-(3-hydroxy-2-pyridinyl)-4-oxo-2-thioxo-1,2,3,4-tetrahydro-7-quinazolinecarboxamide

To a stirred solution of 3-(3-hydroxy-2-pyridinyl)-4-oxo-2-thioxo-1,2,3,4-tetrahydro-7-quinazolinecarboxylic acid (8a, 88 mg, 0.25 mmol, 1 eq.) in dry DMF (3 mL) were added 3-chlorobenzylamine (36 mg, 0.25 mmol, 1. eq.), DIEA (0.05 mL, 0.275 mmol, 1.1 eq.), and HATU (105 mg, 0.275 mmol, 1.1 eq.) in order. The mixture was heated in microwave at 150° C. for 3 minutes and then purified on HPLC under acidic condition (10-55% gradient). Yield: 6 mg, 3%; LC/MS: M+1=439; ¹H-NMR (400 MHz, DMSO-d₆) δ ppm: 13.25 (br. s., 1H), 10.15 (s, 1H), 9.41 (t, J=6.06 Hz, 1H), 8.06 (d, J=8.08 Hz, 1H), 8.02 (t, J=3.03 Hz, 1H), 7.90 (s, 1H), 7.79 (d, J=8.08 Hz, 1H), 7.43-7.29 (m, 6H), 4.51 (d, J=6.06 Hz, 2H)

### Example 9

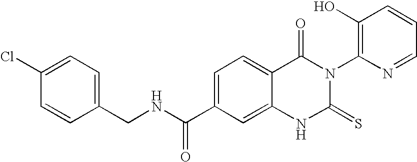

### N-[4-Chlorobenzyl]-3-(3-hydroxy-2-pyridinyl)-4-oxo-2-thioxo-1,2,3,4-tetrahydro-7-quinazolinecarboxamide

To a stirred solution of 3-(3-hydroxy-2-pyridinyl)-4-oxo-2-thioxo-1,2,3,4-tetrahydro-7-quinazolinecarboxylic acid (8a, 88 mg, 0.25 mmol, 1 eq.) in dry DMF (3 mL) were added 4-chlorobenzylamine (36 mg, 0.25 mmol, 1. eq.), DIEA (0.05 mL, 0.275 mmol, 1.1 eq.), and HATU (105 mg, 0.275 mmol, 1.1 eq.) in order. The mixture was heated in microwave at 150° C. for 3 minutes and then purified on HPLC under acidic condition (10-55% gradient). Yield: 24 mg, 22%; LC/MS: M+1=439; ¹H-NMR (400 MHz, DMSO-d₆) δ ppm: 10.13 (br. s., 1H), 9.39 (t, J=6.06 1H), 8.04 (d, J=8.08 Hz, 1H), 8.01 (t, J=3.03 Hz, 1H), 7.89 (s, 1H), 7.77 (d, J=8.08 Hz, 1H), 7.45-7.35 (m, 4H), 7.34 (d, J=3.03 Hz, 2H), 4.49 (d, J=5.56 Hz, 2H)

### Example 10

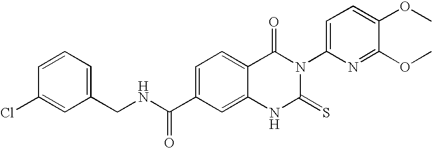

### N-[3-Chlorobenzyl]-3-[5,6-dimethoxy-2-pyridinyl]-4-oxo-2-thioxo-1,2,3,4-tetrahydro-7-quinazolinecarboxamide

### 10a) 3-[5,6-Dimethoxy-2-pyridinyl]-4-oxo-2-thioxo-1,2,3,4-tetrahydro-7-quinazolinecarboxylic acid

To a solution of dimethyl 2-isothiocyanato-1,4-benzenedicarboxylate (1a, 1.03 g, 4.1 mmol, 1 eq.) in DMF (5 mL) was added 5,6-dimethoxy-2-pyridinamine (632 mg, 4.1 mmol, 1 eq.) and the mixture was stirred at rt for 2 hrs. 10% sodium hydroxide (5 mL) was added and the mixture was stirred at 50° C. for one hour. The cooled mixture was acidified with 6N hydrochloric acid to pH 6-7 and the resulting solid was collected by filtration. Yield: 1.45 g, 98%; LC/MS: M+1=356; ¹H NMR (400 MHz, DMSO-d₆) δ ppm 3.78 (s, 3H) 3.86 (s, 3H) 7.01 (d, J=8.08 Hz, 1H) 7.45 (d, J=8.08 Hz, 1H) 7.79 (d, J=9.60 Hz, 1H) 7.91-7.97 (m, 2H) 13.15 (br. s., 1H)

### 10b) N-[3-Chlorobenzyl]-3-[5,6-dimethoxy-2-pyridinyl]-4-oxo-2-thioxo-1,2,3,4-tetrahydro-7-quinazolinecarboxamide

To a stirred solution of 3-[5,6-dimethoxy-2-pyridinyl]-4-oxo-2-thioxo-1,2,3,4-tetrahydro-7-quinazolinecarboxylic acid (10a, 180 mg, 0.5 mmol, 1 eq.) in dry DMF (3 mL) were added 3-chlorobenzylamine (71 mg, 0.5 mmol, 1. eq.), DIEA (0.1 mL, 0.55 mmol, 1.1 eq.), and HATU (209 mg, 0.55 mmol, 1.1 eq.) in order. The mixture was stirred at rt for 1 hour and then purified on HPLC under acidic condition (20-70% gradient). Yield: 101 mg, 42%; LC/MS: M+1=483; ¹H NMR (400 MHz, DMSO-d₆) δ ppm 3.78 (s, 3H) 3.86 (s, 3H) 4.50 (d, J=6.06 Hz, 2H) 7.02 (d, J=8.08 Hz, 1H) 7.30-7.43 (m, 4H) 7.46 (d, J=8.08 Hz, 1H) 7.78 (d, J=8.08 Hz, 1H) 7.89 (s, 1H) 8.05 (d, J=8.08 Hz, 1H) 9.41 (t, J=6.06 Hz, 1H) 13.23 (s, 1H)

### Example 11

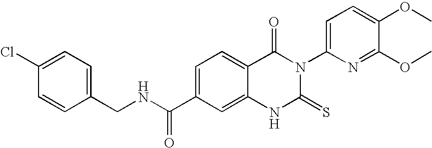

### N-[4-Chlorobenzyl]-3-[5,6-dimethoxy-2-pyridinyl]-4-oxo-2-thioxo-1,2,3,4-tetrahydro-7-quinazolinecarboxamide

To a stirred solution of 3-[5,6-dimethoxy-2-pyridinyl]-4-oxo-2-thioxo-1,2,3,4-tetrahydro-7-quinazolinecarboxylic acid (10a, 180 mg, 0.5 mmol, 1 eq.) in dry DMF (3 mL) were added 4-chlorobenzylamine (71 mg, 0.5 mmol, 1. eq.), DIEA (0.1 mL, 0.55 mmol, 1.1 eq.), and HATU (209 mg, 0.55 mmol, 1.1 eq.) in order. The mixture was stirred at rt for 1 hour and then purified on HPLC under acidic condition (20-70% gradient). Yield: 163 mg, 67%; LC/MS: M+1=483; 1H NMR (400 MHz, DMSO-d₆) δ ppm 3.78 (s, 3H) 3.86 (s, 3H) 4.48 (d, J=5.56 Hz, 2H) 7.02 (d, J=8.08 Hz, 1H) 7.35-7.44 (m, 4H) 7.46 (d, J=8.08 Hz, 1H) 7.77 (d, J=8.08 Hz, 1H) 7.89 (s, 1H) 8.05 (d, J=8.08 Hz, 1H) 9.40 (t, J=6.06 Hz, 1H) 13.23 (s, 1H)

### Example 12

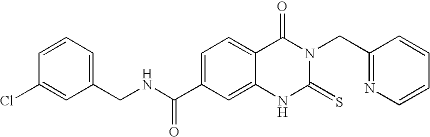

### N-[3-Chlorobenzyl]-4-oxo-3-(2-pyridinylmethyl)-2-thioxo-1,2,3,4-tetrahydro-7-quinazolinecarboxamide

### 12a) 4-oxo-3-(2-pyridinylmethyl)-2-thioxo-1,2,3,4-tetrahydro-7-quinazolinecarboxylic acid

To a solution of dimethyl 2-isothiocyanato-1,4-benzenedicarboxylate (1a, 1.005 g, 4 mmol, 1 eq.) in DMF (5 mL) was added 2-aminomethylpyridine (432 mg, 4 mmol, 1 eq.) and the mixture was stirred at rt for 1 hour. 10% sodium hydroxide (5 mL) was added and the mixture was stirred at 50° C. for 30 min. The cooled mixture was acidified with 6N hydrochloric acid to pH 6-7 and the resulting solid was collected by filtration. Yield: 1.005 g, 80; % LC/MS: M+1=314; 1H NMR (400 MHz, DMSO-d₆δ ppm 5.76 (s, 2H) 7.24 (dd, J=7.07, 5.05 Hz, 1H) 7.31 (d, J=7.58 Hz, 1H) 7.70-7.76 (m, 1H) 7.83 (d, J=8.08 Hz, 1H) 8.01 (s, 1H) 8.06 (d, J=8.08 Hz, 1H) 8.42 (d, J=4.04 Hz, 1H) 13.24 (s, 1H) 13.66 (br. s., 1H)

### 12b) N-[3-Chlorobenzyl]-4-oxo-3-(2-pyridinylmethyl)-2-thioxo-1,2,3,4-tetrahydro-7-quinazolinecarboxamide

To a stirred solution of 4-oxo-3-(2-pyridinylmethyl)-2-thioxo-1,2,3,4-tetrahydro-7-quinazolinecarboxylic acid (12a, 157 mg, 0.5 mmol, 1 eq.) in dry DMF (3 mL) were added 3-chlorobenzylamine (71 mg, 0.5 mmol, 1. eq.), DIEA (0.1 mL, 0.55 mmol, 1.1 eq.), and HATU (209 mg, 0.55 mmol, 1.1 eq.) in order. The mixture was stirred at rt for 1 hour and then purified on HPLC under acidic condition (15-65% gradient). Yield: 43 mg, 20%; LC/MS: M+1=437; 1H NMR (400 MHz, DMSO-d₆) δ ppm 4.50 (d, J=6.06 Hz, 2H) 5.76 (s, 2H) 7.25 (m, 1H) 7.29-7.43 (m, 5H) 7.70-7.76 (m, 1H) 7.78 (d, J=8.08 Hz, 1H) 7.89 (s, 1H) 8.06 (d, J=8.59 Hz, 1H) 8.43 (d, J=4.04 Hz, 1H) 9.39 (t, J=5.81 Hz, 1H) 13.23 (s, 1H)

### Example 13

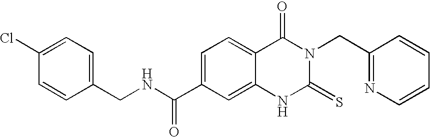

### N-[4-Chlorobenzyl]-4-oxo-3-(2-pyridinylmethyl)-2-thioxo-1,2,3,4-tetrahydro-7-quinazolinecarboxamide

To a stirred solution of 4-oxo-3-(2-pyridinylmethyl)-2-thioxo-1,2,3,4-tetrahydro-7-quinazolinecarboxylic acid (12a, 157 mg, 0.5 mmol, 1 eq.) in dry DMF (3 mL) were added 4-chlorobenzylamine (71 mg, 0.5 mmol, 1. eq.), DIEA (0.1 mL, 0.55 mmol, 1.1 eq.), and HATU (209 mg, 0.55 mmol, 1.1 eq.) in order. The mixture was stirred at rt for 1 hour and then purified on HPLC under acidic condition (15-65% gradient). Yield: 50 mg, 23%; LC/MS: M+1=437; 1H NMR (400 MHz, DMSO-d₆) δ ppm 4.49 (d, J=5.56 Hz, 2H) 5.76 (s, 2H) 7.24 (m, 1H) 7.31 (d, J=8.08 Hz, 1H) 7.34-7.44 (m, 4H) 7.78 (d, J=8.08 Hz, 1H) 7.73 (t, J=6.82 Hz, 1H) 7.88 (s, 1H) 8.05 (d, J=8.08 Hz, 1H) 8.42 (d, J=4.55 Hz, 1H) 9.38 (t, J=6.06 Hz, 1H) 13.22 (s, 1H)

### Example 14

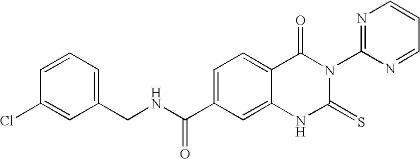

### N-[3-Chlorobenzyl]-4-oxo-3-(2-pyrimidinyl)-2-thioxo-1,2,3,4-tetrahydro-7-quinazolinecarboxamide

### 14a) 4-Oxo-3-(2-pyrimidinyl)-2-thioxo-1,2,3,4-tetrahydro-7-quinazolinecarboxylic acid

To a solution of dimethyl 2-isothiocyanato-1,4-benzenedicarboxylate (1a, 1.005 g, 4 mmol, 1 eq.) in DMF (8 mL) was added 2-amino-pyrimidine (380 mg, 4 mmol, 1 eq.) and the mixture was stirred at rt for 1 hour and then at 50° C. overnight. 10% sodium hydroxide (1 mL) was added and the mixture was stirred at rt for 2 hours. More 10% sodium hydroxide (1 mL) was added and the mixture was stirred at rt over the weekend. More 10% sodium hydroxide (2 mL) was added and the mixture was stirred at room temperature for another 3 h. The mixture was acidified with 6N hydrochloric acid to pH 6 and let stirred at rt overnight. The resulting solid was collected, washed with water, and dried in a vacuum oven at 50° C. for 2 hours. To the solid was added 1 N hydrochloric acid (3 mL) and the mixture was stirred for 1 hour. The remaining solid was then collected by filtration to give the desired pure product. Yield: 327 mg, 27%; LC/MS: M+1=301; 1H NMR (400 MHz, DMSO-d₆) δ ppm 7.68 (t, J=4.93 Hz, 1H) 7.87 (dd, J=8.34, 1.52 Hz, 1H) 8.04 (d, J=1.26 Hz, 1H) 8.10 (d, J=8.34 Hz, 1H) 9.04 (d, J=4.80 Hz, 2H) 13.46 (s, 1H) 13.72 (br. s., 1H)

### 14b) N-[3-Chlorobenzyl]-4-oxo-3-(2-pyrimidinyl)-2-thioxo-1,2,3,4-tetrahydro-7-quinazolinecarboxamide

To a stirred solution of 4-oxo-3-(2-pyrimidinyl)-2-thioxo-1,2,3,4-tetrahydro-7-quinazolinecarboxylic acid (14a, 150 mg, 0.5 mmol, 1 eq.) in dry DMF (3 mL) were added 3-chlorobenzylamine (71 mg, 0.5 mmol, 1. eq.), DIEA (0.1 mL, 0.55 mmol, 1.1 eq.), and HATU (209 mg, 0.55 mmol, 1.1 eq.) in order. The mixture was stirred at rt for 1 hour and then purified on HPLC under acidic condition (15-65% gradient). Yield: 25 mg, 12%; LC/MS: M+1=424; 1H NMR (400 MHz, DMSO-d₆) δ ppm 4.51 (d, J=5.81 Hz, 2H) 7.29-7.43 (m, 4H) 7.68 (t, J=4.93 Hz, 1H) 7.82 (dd, J=8.34, 1.52 Hz, 1H) 7.92 (d, J=1.26 Hz, 1H) 8.10 (d, J=8.34 Hz, 1H) 9.04 (d, J=5.05 Hz, 2H) 9.44 (t, J=5.94 Hz, 1H) 13.46 (s, 1H)

### Example 15

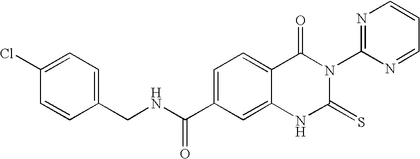

### N-[4-Chlorobenzyl]-4-oxo-3-(2-pyrimidinyl)-2-thioxo-1,2,3,4-tetrahydro-7-quinazolinecarboxamide

To a stirred solution of 4-oxo-3-(2-pyrimidinyl)-2-thioxo-1,2,3,4-tetrahydro-7-quinazolinecarboxylic acid (14a, 150 mg, 0.5 mmol, 1 eq.) in dry DMF (3 mL) were added 4-chlorobenzylamine (71 mg, 0.5 mmol, 1. eq.), DIEA (0.1 mL, 0.55 mmol, 1.1 eq.), and HATU (209 mg, 0.55 mmol, 1.1 eq.) in order. The mixture was stirred at rt for 1 hour and then purified on HPLC under acidic condition (15-65% gradient). Yield: 116 mg, 55%; LC/MS: M+1=424; 1H NMR (400 MHz, DMSO-d₆) δ ppm 4.49 (d, J=6.06 Hz, 2H) 7.35-7.45 (m, 4H) 7.68 (t, J=4.93 Hz, 1H) 7.81 (dd, J=8.34, 1.52 Hz, 1H) 7.92 (d, J=1.26 Hz, 1H) 8.09 (d, J=8.34 Hz, 1H) 9.03 (d, J=4.80 Hz, 2H) 9.43 (t, J=5.94 Hz, 1H) 13.46 (s, 1H)

### Example 16

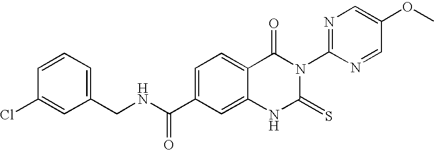

### N-[3-Chlorobenzyl]-3-[5-methoxy-2-pyrimidinyl]-4-oxo-2-thioxo-1,2,3,4-tetrahydro-7-quinazolinecarboxamide

### 16a) 3-[5-Methoxy-2-pyrimidinyl]-4-oxo-2-thioxo-1,2,3,4-tetrahydro-7-quinazolinecarboxylic acid

To a solution of dimethyl 2-isothiocyanato-1,4-benzenedicarboxylate (1a, 1.005 g, 4 mmol, 1 eq.) in DMF (6 mL) was added 5-methoxy-2-pyrimidinamine (480 mg, 4 mmol, 1 eq.) and the mixture was stirred at rt overnight (solid gradually precipitated out during the course). 10% sodium hydroxide (5 mL) was added (the mixture became clear) and it was stirred at rt for 4 hours and then at 50° C. for 2 hours (monitored with LC/MS). The cooled mixture was acidified with 6N hydrochloric acid to pH 6 and stirred at rt overnight. The resulting solid was collected, washed with water, and dried in vacuum oven at 50° C. for 2 hours to give 533 mg of the product. The filtrate was concentrated and then suspended in water. The remaining solid was collected and dried to give another 622 mg of the product. Both solids were combined and its LC/MS showed >95% purity while as its NMR showed ˜50% purity (mixture of the desired product and the starting aminopyrimidine in almost 1:1 ratio). This material was taken forward without further purification. Yield: 1.155 g, 87% (˜45% in the consideration of the impurity); LC/MS: M+1=331; 1H NMR (400 MHz, DMSO-d₆) δ ppm 4.01 (s, 3H) 7.86 (dd, J=8.08, 1.52 Hz, 1H) 8.04 (d, 1H) 8.09 (d, J=8.34 Hz, 1H) 8.74 (s, 2H) 13.42 (br. s., 11H) 13.72 (br. s., 1H)

### 16b) N-[3-Chlorobenzyl]-3-[5-methoxy-2-pyrimidinyl]-4-oxo-2-thioxo-1,2,3,4-tetrahydro-7-quinazolinecarboxamide

To a stirred solution of 3-[5-methoxy-2-pyrimidinyl]-4-oxo-2-thioxo-1,2,3,4-tetrahydro-7-quinazolinecarboxylic acid (16a, 165 mg, 0.5 mmol, 1 eq.) in dry DMF (3 mL) were added 3-chlorobenzylamine (71 mg, 0.5 mmol, 1. eq.), DIEA (0.1 mL, 0.55 mmol, 1.1 eq.), and HATU (209 mg, 0.55 mmol, 1.1 eq.) in order. The mixture was stirred at rt for 1 hour and then purified on HPLC under acidic condition (15-65% gradient). Yield: 88 mg, 38%; LC/MS: M+1=454; 1H NMR (400 MHz, DMSO-d₆) δ ppm 4.01 (s, 3H) 4.51 (d, J=6.06 Hz, 2H) 7.29-7.45 (m, 4H) 7.81 (d, J=8.59 Hz, 1H) 7.91 (s, 1H) 8.09 (d, J=8.08 Hz, 1H) 8.74 (s, 2H) 9.43 (t, J=6.06 Hz, 1H) 13.41 (s, 1H)

### Example 17

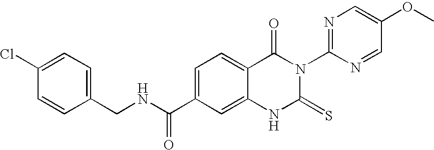

### N-[4-Chlorobenzyl]-3-[5-methoxy-2-pyrimidinyl]-4-oxo-2-thioxo-1,2,3,4-tetrahydro-7-quinazolinecarboxamide

To a stirred solution of 3-[5-methoxy-2-pyrimidinyl]-4-oxo-2-thioxo-1,2,3,4-tetrahydro-7-quinazolinecarboxylic acid (16a, 165 mg, 0.5 mmol, 1 eq.) in dry DMF (3 mL) were added 4-chlorobenzylamine (71 mg, 0.5 mmol, 1. eq.), DIEA (0.1 mL, 0.55 mmol, 1.1 eq.), and HATU (209 mg, 0.55 mmol, 1.1 eq.) in order. The mixture was stirred at rt for 1 hour and then purified on HPLC under acidic condition (15-65% gradient). Yield: 116 mg, 51%; LC/MS: M+1=454; 1H NMR (400 MHz, DMSO-d₆) δ ppm 4.01 (s, 3H) 4.49 (d, J=6.06 Hz, 2H) 7.33-7.45 (m, 4H) 7.81 (d, J=8.08 Hz, 1H) 7.91 (s, 1H) 8.09 (d, J=8.08 Hz, 1H) 8.73 (s, 2H) 9.42 (t, J=5.81 Hz, 1H) 13.41 (s, 1H)

### Example 18

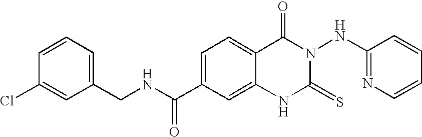

### N-[3-Chlorobenzyl]-4-oxo-3-(2-pyridinylamino)-2-thioxo-1,2,3,4-tetrahydro-7-quinazolinecarboxamide

### 18a) 4-Oxo-3-(2-pyridinylamino)-2-thioxo-1,2,3,4-tetrahydro-7-quinazolinecarboxylic acid

To a solution of dimethyl 2-isothiocyanato-1,4-benzenedicarboxylate (1a, 1.005 g, 4 mmol, 1 eq.) in DMF (5 mL) was added 2-hydrazinopyridine (436 mg, 4 mmol, 1 eq.) and the mixture was stirred at rt for 1 hour. 10% sodium hydroxide (5 mL) was added and the mixture was stirred at 50° C. for 1 hour. The cooled mixture was acidified with 6N hydrochloric acid to pH 6 and let stirred at rt overnight. The resulting solid was collected, washed with water, and dried in vacuum oven at 50° C. for 2 hours. Yield: 781 mg, 62%; LC/MS: M+1=315; 1H NMR (400 MHz, DMSO-d₆) δ ppm 6.77 (m, 1H) 6.80 (d, J=8.59 Hz, 1H) 7.58 (t, J=7.07 Hz, 1H) 7.83 (d, J=8.59 Hz, 1H) 7.93-7.99 (m, 1H) 8.01 (s, 1H) 8.06 (d, J=8.08 Hz, 1H) 9.51 (s, 1H) 13.25 (br. s., 1H) 13.71 (br. s., 1H)

### 18b) N-[3-Chlorobenzyl]-4-oxo-3-(2-pyridinylamino)-2-thioxo-1,2,3,4-tetrahydro-7-quinazolinecarboxamide

To a stirred solution of 4-oxo-3-(2-pyridinylamino)-2-thioxo-1,2,3,4-tetrahydro-7-quinazolinecarboxylic acid (18a, 157 mg, 0.5 mmol, 1 eq.) in dry DMF (3 mL) were added 3-chlorobenzylamine (71 mg, 0.5 mmol, 1. eq.), DIEA (0.1 mL, 0.55 mmol, 1.1 eq.), and HATU (209 mg, 0.55 mmol, 1.1 eq.) in order. The mixture was stirred at rt for 1 hour and then purified on HPLC under acidic condition (15-55% gradient) to give the product as its TFA salt. Yield: 70 mg, 25%; LC/MS: M+1=438; 1H NMR (400 MHz, DMSO-d₆) δ ppm 4.51 (d, J=6.06 Hz, 2H) 6.98 (br. s., 1H) 7.11 (br. s., 1H) 7.29-7.44 (m, 4H) 7.81 (d, J=8.08 Hz, 1H) 7.90 (s, 2H) 8.04-8.11 (m, 2H) 9.42 (t, J=5.81 Hz, 1H) 13.41 (br. s., 1H)

### Example 19

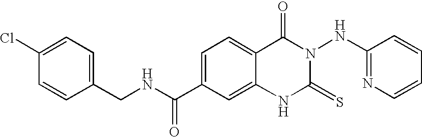

### N-[4-Chlorobenzyl]-4-oxo-3-(2-pyridinylamino)-2-thioxo-1,2,3,4-tetrahydro-7-quinazolinecarboxamide

To a stirred solution of 4-oxo-3-(2-pyridinylamino)-2-thioxo-1,2,3,4-tetrahydro-7-quinazolinecarboxylic acid (18a, 157 mg, 0.5 mmol, 1 eq.) in dry DMF (3 mL) were added 4-chlorobenzylamine (71 mg, 0.5 mmol, 1. eq.), DIEA (0.1 mL, 0.55 mmol, 1.1 eq.), and HATU (209 mg, 0.55 mmol, 1.1 eq.) in order. The mixture was stirred at rt for 1 hour and then purified on HPLC under acidic condition (15-55% gradient) to give the product as its TFA salt. Yield: 82 mg, 30%; LC/MS: M+1=438; 1H NMR (400 MHz, DMSO-d₆) δ ppm 4.49 (d, J=6.06 Hz, 2H) 6.99 (br. s., 1H) 7.13 (br. s., 1H) 7.34-7.45 (m, 4H) 7.81 (d, J=8.08 Hz, 1H) 7.90 (s, 2H) 8.04-8.11 (m, 2H) 9.41 (t, J=6.06 Hz, 1H) 13.41 (br. s., 1H)

### Example 20

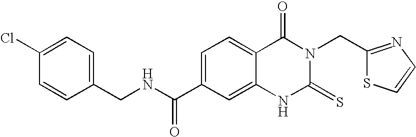

### N-[4-Chlorobenzyl]-4-oxo-3-(1,3-thiazol-2-ylmethyl)-2-thioxo-1,2,3,4-tetrahydro-7-quinazolinecarboxamide

### 20a) 4-Oxo-3-(1,3-thiazol-2-ylmethyl)-2-thioxo-1,2,3,4-tetrahydro-7-quinazolinecarboxylic acid

To a solution of dimethyl 2-isothiocyanato-1,4-benzenedicarboxylate (1a, 1.005 g, 4 mmol, 1 eq.) in DMF (6 mL) was added 1-(1,3-thiazol-2-yl)methylamine (547 mg, 4 mmol, 1 eq.) and the mixture was stirred at rt for 1 hour. 10% sodium hydroxide (5 mL) was added and the mixture was stirred at 50° C. for 1 hour. The cooled mixture was acidified with 6N hydrochloric acid to pH 6 and stirred at rt overnight. The resulting solid was collected, washed with water, and dried in vacuum oven at 50° C. for 2 hours. Yield: 1.22 g, 96%; LC/MS: M+1=320; 1H NMR (400 MHz, DMSO-d₆δ ppm 5.95 (s, 2H) 7.65 (d, J=3.03 Hz, 1H) 7.70 (d, J=3.54 Hz, 1H) 7.84 (d, J=8.59 Hz, 1H) 8.00 (s, 1H) 8.08 (d, J=8.08 Hz, 1H) 13.33 (br. s., 1H) 13.72 (br. s., 1H)

### 20b) N-[4-Chlorobenzyl]-4-oxo-3-(1,3-thiazol-2-ylmethyl)-2-thioxo-1,2,3,4-tetrahydro-7-quinazolinecarboxamide

To a stirred solution of 4-oxo-3-(1,3-thiazol-2-ylmethyl)-2-thioxo-1,2,3,4-tetrahydro-7-quinazolinecarboxylic acid (20a, 120 mg, 0.375 mmol, 1 eq.) in dry DMF (2 mL) were added 4-chlorobenzylamine (53 mg, 0.375 mmol, 1. eq.), DIEA (0.07 mL, 0.413 mmol, 1.1 eq.), and HATU (157 mg, 0.413 mmol, 1.1 eq.) in order. The mixture was stirred at rt for 1 hour, the product precipitated. The solid was collected by filtration and then suspended in methanol to which was added 4M hydrogen chloride in dioxane (0.25 mL, 2 eq.). The mixture was stirred for another 30 min and the resulting solid was collected and washed with ethanol. It was resuspended in methanol and let stand for 1 hour. The remaining solid was collected and washed once more with methanol to give 68 mg of pure product as its HCl salt. Yield: 61 mg, 38%; LC/MS: M+1=443; 1H NMR (400 MHz, DMSO-d₆) δ ppm 4.48 (d, J=5.56 Hz, 2H) 5.95 (s, 2H) 7.33-7.44 (m, 4H) 7.62-7.66 (m, 1H) 7.70 (d, J=3.03 Hz, 1H) 7.78 (d, J=8.08 Hz, 1H) 7.87 (s, 1H) 8.07 (d, J=8.08 Hz, 1H) 9.39 (t, J=5.81 Hz, 1H) 13.32 (s, 1H)

### Example 21

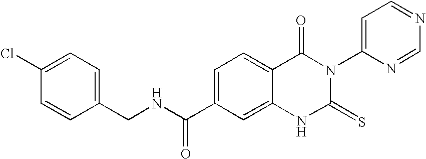

### N-[4-Chlorobenzyl]-4-oxo-3-(4-pyrimidinyl)-2-thioxo-1,2,3,4-tetrahydro-7-quinazolinecarboxamide

### 21a) 4-Oxo-3-(4-pyrimidinyl)-2-thioxo-1,2,3,4-tetrahydro-7-quinazolinecarboxylic acid

To a solution of dimethyl 2-isothiocyanato-1,4-benzenedicarboxylate (1a, 1.005 g, 4 mmol, 1 eq.) in DMF (6 mL) was added 4-pyrimidinamine (380 mg, 4 mmol, 1 eq.) and the mixture was stirred at rt for 1 hour. 10% sodium hydroxide (5 mL) was added and the mixture was stirred at 50° C. for 1 hour. The cooled mixture was acidified with 6N hydrochloric acid to pH 6 and let stirred at rt overnight. The resulting solid was collected, washed with water, and dried in vacuum oven at 50° C. for 2 hours. Yield: 278 mg, 23%; LC/MS: M+1=301; 1H NMR (400 MHz, DMSO-d₆) δ ppm 7.77 (d, J=4.04 Hz, 1H) 7.86 (d, J=9.60 Hz, 1H) 8.03 (s, 1H) 8.08 (d, J=8.08 Hz, 1H) 9.07 (d, J=5.05 Hz, 1H) 9.33 (s, 1H) 13.46 (br. s., 1H) 13.68 (br. s., 1H)

### 21b) N-[4-Chlorobenzyl]-4-oxo-3-(4-pyrimidinyl)-2-thioxo-1,2,3,4-tetrahydro-7-quinazolinecarboxamide

To a stirred solution of 4-oxo-3-(4-pyrimidinyl)-2-thioxo-1,2,3,4-tetrahydro-7-quinazolinecarboxylic acid (21a, 135 mg, 0.45 mmol, 1 eq.) in dry DMF (2 mL) were added 4-chlorobenzylamine (64 mg, 0.45 mmol, 1. eq.), DIEA (0.09 mL, 0.495 mmol, 1.1 eq.), and HATU (188 mg, 0.495 mmol, 1.1 eq.) in order. The mixture was stirred at rt for 1 hour and then purified on HPLC under acidic condition (15-65% gradient). Yield: 35 mg, 18%; LC/MS: M+1=424; 1H NMR (400 MHz, DMSO-d₆) δ ppm 4.49 (d, J=6.06 Hz, 2H) 7.34-7.45 (m, 4H) 7.76-7.83 (m, 2H) 7.91 (s, 1H) 8.07 (d, J=8.08 Hz, 1H) 9.06 (d, J=5.05 Hz, 1H) 9.33 (s, 1H) 9.42 (t, J=6.06 Hz, 1H) 13.44 (s, 1H)

### Example 22

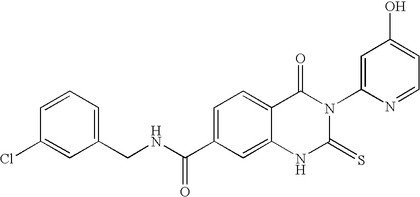

### N-[3-Chlorobenzyl]-3-(4-hydroxy-2-pyridinyl)-4-oxo-2-thioxo-1,2,3,4-tetrahydro-7-quinazolinecarboxamide

### 22a) 3-(4-Hydroxy-2-pyridinyl)-4-oxo-2-thioxo-1,2,3,4-tetrahydro-7-quinazolinecarboxylic acid

To a solution of dimethyl 2-isothiocyanato-1,4-benzenedicarboxylate (1a, 1.005 g, 4 mmol, 1 eq.) in DMF (6 mL) was added 2-amino-4-pyridinol (440 mg, 4 mmol, 1 eq.) and the mixture was stirred at rt overnight. 10% sodium hydroxide (5 mL) was added and the mixture was stirred at rt for 1 hour. The mixture was acidified with 6N hydrochloric acid to pH 6 and stirred at rt for 5 hours. The resulting solid was collected, washed with water, and dried in vacuum oven at 40° C. overnight. Yield: 617 mg, 49%; LC/MS: M+1=316; 1H NMR (400 MHz, DMSO-d₆) δ ppm 6.85 (br. s., 2H) 7.83 (d, J=8.08 Hz, 1H) 8.01 (s, 1H) 8.05 (d, J=8.08 Hz, 1H) 8.25 (m, 1H) 11.05 (br. s., 0.7H) 13.25 (br. s., 1H), 13.58 (br. s., 0.6H)

### 22b) N-[3-Chlorobenzyl]-3-(4-hydroxy-2-pyridinyl)-4-oxo-2-thioxo-1,2,3,4-tetrahydro-7-quinazolinecarboxamide

To a stirred solution of 3-(4-hydroxy-2-pyridinyl)-4-oxo-2-thioxo-1,2,3,4-tetrahydro-7-quinazolinecarboxylic acid (22a, 110 mg, 0.35 mmol, 1 eq.) in dry DMF (2 mL) were added 3-chlorobenzylamine (50 mg, 0.35 mmol, 1. eq.), DIEA (0.07 mL, 0.385 mmol, 1.1 eq.), and HATU (146 mg, 0.385 mmol, 1.1 eq.) in order. The mixture was stirred at rt for 3 hours and then purified on HPLC under acidic condition (10-55% gradient). Yield: 55 mg, 36%; LC/MS: M+1=439; 1H NMR (400 MHz, DMSO-d₆) δ ppm 4.51 (d, J=6.06 Hz, 2H) 6.84-6.90 (m, 2H) 7.29-7.43 (m, 4H) 7.78 (d, J=8.59 Hz, 1H) 7.89 (s, 1H) 8.05 (d, J=8.59 Hz, 1H) 8.27 (d, J=5.56 Hz, 1H) 9.41 (t, J=6.06 Hz, 1H) 13.23 (s, 1H)

### Example 23

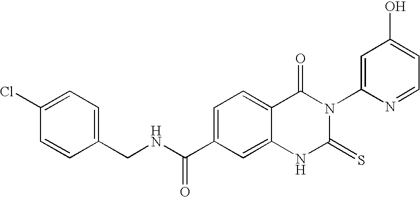

### N-[4-Chlorobenzyl]-3-(4-hydroxy-2-pyridinyl)-4-oxo-2-thioxo-1,2,3,4-tetrahydro-7-quinazolinecarboxamide

To a stirred solution of 3-(4-hydroxy-2-pyridinyl)-4-oxo-2-thioxo-1,2,3,4-tetrahydro-7-quinazolinecarboxylic acid (22a, 110 mg, 0.35 mmol, 1 eq.) in dry DMF (2 mL) were added 4-chlorobenzylamine (50 mg, 0.35 mmol, 1. eq.), DIEA (0.07 mL, 0.385 mmol, 1.1 eq.), and HATU (146 mg, 0.385 mmol, 1.1 eq.) in order. The mixture was stirred at rt for 3 hours and then purified on HPLC under acidic condition (10-55% gradient). Yield: 67 mg, 44%; LC/MS: M+1=439; 1H NMR (400 MHz, DMSO-d₆) δ ppm 4.49 (d, J=6.06 Hz, 2H) 6.87-6.97 (m, 2H) 7.34-7.46 (m, 4H) 7.78 (d, J=8.59 Hz, 1H) 7.89 (s, 1H) 8.05 (d, J=8.08 Hz, 1H) 8.31 (d, J=5.56 Hz, 1H) 9.40 (t, J=5.81 Hz, 1H) 13.26 (s, 1H)

### Example 24

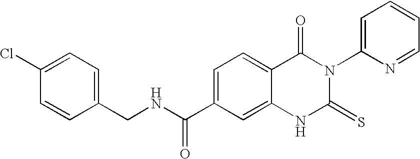

### N-[4-Chlorobenzyl]-3-(2-pyridinyl)-4-oxo-2-thioxo-1,2,3,4-tetrahydro-7-quinazolinecarboxamide

### 24a) 4-Oxo-3-(2-pyridinyl)-2-thioxo-1,2,3,4-tetrahydro-7-quinazolinecarboxylic acid

To a solution of dimethyl 2-isothiocyanato-1,4-benzenedicarboxylate (1a, 1.005 g, 4 mmol, 1 eq.) in DMF (5 mL) was added 2-amino-pyridine (376 mg, 4 mmol, 1 eq.) and the mixture was stirred at rt overnight. 10% sodium hydroxide (5 mL) was added and the mixture was stirred at rt for 1.5 hours. The mixture was acidified with 6N hydrochloric acid to pH 6 and diluted with water. The resulting solid was collected, washed with water, and dried. Yield: 830 mg, 70%; LC/MS: M+1=300; 1H NMR (400 MHz, DMSO-d₆) δ ppm 7.46-7.55 (m, 2H) 7.84 (d, J=9.60 Hz, 1H) 7.97-8.04 (m, 2H) 8.07 (d, J=8.08 Hz, 1H) 8.60 (d, J=3.03 Hz, 1H) 13.30 (s, 1H) 13.69 (br. s., 1H)

### 24b) N-[4-Chlorobenzyl]-3-(2-pyridinyl)-4-oxo-2-thioxo-1,2,3,4-tetrahydro-7-quinazolinecarboxamide

To a stirred solution of 3-(2-pyridinyl)-4-oxo-2-thioxo-1,2,3,4-tetrahydro-7-quinazolinecarboxylic acid (24a, 78 mg, 0.26 mmol, 1 eq.) in dry DMF (2 mL) were added 4-chlorobenzylamine (37 mg, 0.26 mmol, 1. eq.), DIEA (0.05 mL, 0.286 mmol, 1.1 eq.), and HATU (109 mg, 0.286 mmol, 1.1 eq.) in order. The mixture was stirred at rt for one hour and then purified on HPLC under acidic condition (25-55% gradient). Yield: 54 mg, 50%; LC/MS: M+1=423; 1H NMR (400 MHz, DMSO-d₆) δ ppm 4.49 (d, J=6.06 Hz, 2H) 7.35-7.45 (m, 4H) 7.46-7.54 (m, 2H) 7.79 (d, J=8.08 Hz, 1H) 7.90 (s, 1H) 8.00 (td, J=7.58, 2.02 Hz, 1H) 8.06 (d, J=8.08 Hz, 1H) 8.60 (d, J=3.54 Hz, 1H) 9.40 (t, J=6.06 Hz, 1H) 13.29 (s, 1H)

### Example 25

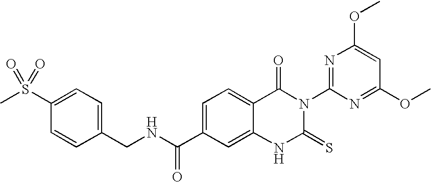

### 3-[4,6-Dimethoxy-2-pyrimidinyl]-N-[4-(methylsulfonyl)benzyl]-4-oxo-2-thioxo-1,2,3,4-tetrahydro-7-quinazolinecarboxamide

To a stirred solution of 3-[4,6-dimethoxy-2-pyrimidinyl]-4-oxo-2-thioxo-1,2,3,4-tetrahydro-7-quinazolinecarboxylic acid (4a, 80 mg, 0.22 mmol, 1 eq.) in dry DMF (2 mL) were added 4-(methylsulfonyl)benzylamine hydrochloride (49 mg, 0.22 mmol, 1 eq.), DIEA (0.09 mL, 0.5 mmol, 2.2 eq.), and HATU (91 mg, 0.24 mmol, 1.1 eq.) in order. The mixture was stirred at rt for 1 hour and then purified on HPLC under acidic condition (30-60% gradient). Yield: 58 mg, 50%; LC/MS: M+1=528; 1H NMR (400 MHz, DMSO-d₆) δ ppm 3.20 (s, 3H) 3.89 (s, 6H) 4.60 (d, J=6.06 Hz, 2H) 6.45 (s, 1H) 7.61 (d, J=8.59 Hz, 2H) 7.83 (d, J=8.59 Hz, 1H) 7.91 (d, J=8.08 Hz, 3H) 8.10 (d, J=8.08 Hz, 1H) 9.53 (t, J=5.81 Hz, 1H) 13.44 (s, 1H)

### Example 26

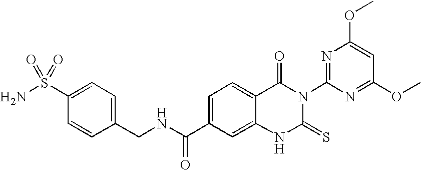

### N-[4-(Aminosulfonyl)benzyl]-3-[4,6-dimethoxy-2-pyrimidinyl]-4-oxo-2-thioxo-1,2,3,4-tetrahydro-7-quinazolinecarboxamide

To a stirred solution of 3-[4,6-dimethoxy-2-pyrimidinyl]-4-oxo-2-thioxo-1,2,3,4-tetrahydro-7-quinazolinecarboxylic acid (4a, 80 mg, 0.22 mmol, 1 eq.) in dry DMF (2 mL) were added 4-(aminosulfonyl)benzylamine (41 mg, 0.22 mmol, 1 eq.), DIEA (0.09 mL, 0.5 mmol, 2.2 eq.), and HATU (91 mg, 0.24 mmol, 1.1 eq.) in order. The mixture was stirred at rt for 1 hour and then purified on HPLC under acidic condition (28-58% gradient). Yield: 49 mg, 42%; LC/MS: M+1=529; 1H NMR (400 MHz, DMSO-d₆) δ ppm 3.89 (s, 6H) 4.57 (d, J=6.06 Hz, 2H) 6.45 (s, 1H) 7.34 (s, 2H) 7.52 (d, J=8.59 Hz, 2H) 7.77-7.85 (m, 3H) 7.91 (s, 1H) 8.09 (d, J=8.08 Hz, 1H) 9.49 (t, J=5.81 Hz, 1H) 13.43 (s, 1H)

### Example 27

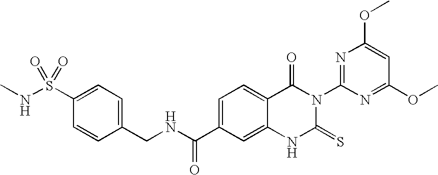

### 3-[4,6-Dimethoxy-2-pyrimidinyl]-N-[4-(methylaminosulfonyl)benzyl]-4-oxo-2-thioxo-1,2,3,4-tetrahydro-7-quinazolinecarboxamide

To a stirred solution of 3-[4,6-dimethoxy-2-pyrimidinyl]-4-oxo-2-thioxo-1,2,3,4-tetrahydro-7-quinazolinecarboxylic acid (4a, 80 mg, 0.22 mmol, 1 eq.) in dry DMF (2 mL) were added 4-(methylaminosulfonyl)benzylamine (44 mg, 0.22 mmol, 1 eq.), DIEA (0.09 mL, 0.5 mmol, 2.2 eq.), and HATU (91 mg, 0.24 mmol, 1.1 eq.) in order. The mixture was stirred at rt for 1 hour and then purified on HPLC under acidic condition (30-60% gradient). Yield: 48 mg, 40%; LC/MS: M+1=543; 1H NMR (400 MHz, DMSO-d₆) δ ppm 2.40 (d, J=5.05 Hz, 3H) 3.89 (s, 6H) 4.59 (d, J=6.06 Hz, 2H) 6.45 (s, 1H) 7.44 (q, J=5.05 Hz, 1H) 7.56 (d, J=8.59 Hz, 2H) 7.76 (d, J=8.59 Hz, 2H) 7.84 (d, J=8.08 Hz, 1H) 7.92 (s, 1H) 8.09 (d, J=8.08 Hz, 1H) 9.50 (t, J=5.81 Hz, 1H) 13.44 (s, 1H)

### Example 28

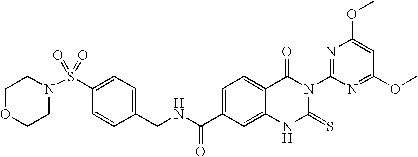

### 3-[4,6-Dimethoxy-2-pyrimidinyl]-N-[4-(morpholinylsulfonyl)benzyl]-4-oxo-2-thioxo-1,2,3,4-tetrahydro-7-quinazolinecarboxamide

To a stirred solution of 3-[4,6-dimethoxy-2-pyrimidinyl]-4-oxo-2-thioxo-1,2,3,4-tetrahydro-7-quinazolinecarboxylic acid (4a, 80 mg, 0.22 mmol, 1 eq.) in dry DMF (2 mL) were added 4-(morpholinylsulfonyl)benzylamine hydrochloride (64 mg, 0.22 mmol, 1 eq.), DIEA (0.09 mL, 0.5 mmol, 2.2 eq.), and HATU (91 mg, 0.24 mmol, 1.1 eq.) in order. The mixture was stirred at rt for 1 hour and then purified on HPLC under acidic condition (35-65% gradient). Yield: 82 mg, 62%; LC/MS: M+1=599; 1H NMR (400 MHz, DMSO-d₆) δ ppm 2.81-2.88 (m, 4H) 3.64 (br. s., 5H) 3.59-3.66 (m, 4H) 3.89 (s, 6H) 4.63 (d, J=6.06 Hz, 2H) 6.45 (s, 1H) 7.60-7.65 (m, 2H) 7.71-7.76 (m, 2H) 7.85 (d, J=8.59 Hz, 1H) 7.93 (s, 1H) 8.10 (d, J=8.08 Hz, 1H) 9.52 (t, J=6.06 Hz, 1H) 13.45 (s, 1H)

### Example 29

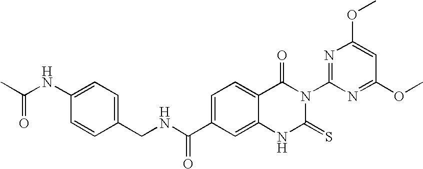

### N-[4-(Acetylamino)benzyl]-3-[4,6-dimethoxy-2-pyrimidinyl]-4-oxo-2-thioxo-1,2,3,4-tetrahydro-7-quinazolinecarboxamide

To a stirred solution of 3-[4,6-dimethoxy-2-pyrimidinyl]-4-oxo-2-thioxo-1,2,3,4-tetrahydro-7-quinazolinecarboxylic acid (4a, 80 mg, 0.22 mmol, 1 eq.) in dry DMF (2 mL) were added 4-(acetylamino)benzylamine (36 mg, 0.22 mmol, 1 eq.), DIEA (0.09 mL, 0.5 mmol, 2.2 eq.), and HATU (91 mg, 0.24 mmol, 1.1 eq.) in order. The mixture was stirred at rt for 1 hour and then purified on HPLC under acidic condition (28-58% gradient). Yield: 43 mg, 38%; LC/MS: M+1=507; 1H NMR (400 MHz, DMSO-d₆) δ ppm 2.03 (s, 3H) 3.89 (s, 6H) 4.44 (d, J=5.56 Hz, 2H) 6.45 (s, 1H) 7.26 (d, J=8.59 Hz, 2H) 7.54 (d, J=8.59 Hz, 2H) 7.80 (d, J=8.08 Hz, 1H) 7.90 (s, 1H) 8.07 (d, J=8.08 Hz, 1H) 9.34 (t, J=5.81 Hz, 1H) 9.94 (s, 1H) 13.43 (s, 1H)

### Example 30

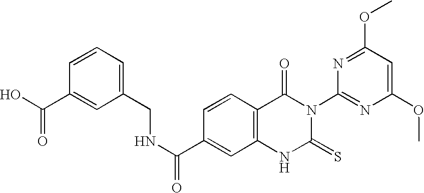

### 3-{[({3-[4,6-Dimethoxy-2-pyrimidinyl]-4-oxo-2-thioxo-1,2,3,4-tetrahydro-7-quinazolinyl}carbonyl)amino]methyl}benzoic acid

To a stirred solution of 3-[4,6-dimethoxy-2-pyrimidinyl]-4-oxo-2-thioxo-1,2,3,4-tetrahydro-7-quinazolinecarboxylic acid (4a, 200 mg, 0.556 mmols, 1.0 eq.) in dry DMF (4 mL) were added HATU (233 mg, 0.61 mmol, 1.1 eq.) and DIEA (0.212 mL, 1.22 mmol, 2.2 eq.). The solution was stirred at rt for 30 minutes. To this solution was added methyl 3-(aminomethyl)benzoate hydrochloride (110 mg, 0.556 mmol, 1.0 eq.), and was stirred at rt overnight. The solvent was removed by rotary evaporation, and the crude product dissolved in ethyl acetate and washed three times with brine. The solvent was removed by rotary evaporation, and the crude product was dissolved in methanol (5 mL). 10N sodium hydroxide (0.50 mL) added with stirring and the reaction stirred for one hour at rt, then acidified to pH 3-4 with 6N hydrochloric acid. The crude product was dissolved in DMSO (1 mL) and purified on HPLC under acidic conditions. Yield: 39 mg, 14%; LC/MS: M+1=493; ¹H NMR (400 MHz, DMSO-d₆) δ ppm 3.89 (s, 6H) 4.56 (d, J=5.81 Hz, 2H) 6.45 (s, 1H) 7.49 (t, J=7.71 Hz, 1H) 7.61 (d, J=8.08 Hz, 1H) 7.83 (dd, J=12.88, 8.08 Hz, 2H) 7.92 (d, J=1.26 Hz, 2H) 8.09 (d, J=8.34 Hz, 1H) 9.48 (t, J=5.81, 1H) 12.97 (br. s., 1H) 13.44 (br. s., 1H)

### Example 31

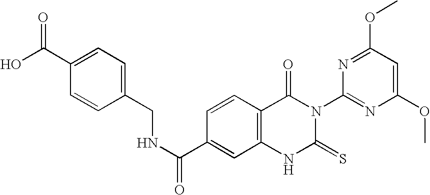

### 4-{[({3-[4,6-Dimethoxy-2-pyrimidinyl]-4-oxo-2-thioxo-1,2,3,4-tetrahydro-7-quinazolinyl}carbonyl)amino]methyl}benzoic acid

To a stirred solution of 3-[4,6-dimethoxy-2-pyrimidinyl]-4-oxo-2-thioxo-1,2,3,4-tetrahydro-7-quinazolinecarboxylic acid (4a, 200 mg, 0.556 mmols, 1.0 eq.) in dry DMF (4 mL) were added HATU (233 mg, 0.61 mmol, 1.1 eq.) and DIEA (0.212 mL, 1.22 mmol, 2.2 eq.). The solution was stirred at rt for 30 minutes. To this solution was added methyl 4-(aminomethyl)benzoate hydrochloride (110 mg, 0.556 mmol, 1.0 eq.), and stirred at rt overnight. The solvent was removed by rotary evaporation, and the crude product was dissolved in ethyl acetate, and washed three times with brine. The solvent was removed by rotary evaporation, and the crude product was dissolved in methanol (5 mL). 10N sodium hydroxide (0.50 mL) was added with stirring and the reaction stirred for one hour at rt then acidified to pH 3-4 with 6M hydrochloric acid. The crude product was dissolved in DMSO (1 mL), and purified on HPLC under acidic conditions. Yield: 12 mg, 4%; LC/MS: M+1=493; ¹H NMR (400 MHz, DMSO-d₆) δ ppm 3.89 (s, 6H) 4.58 (d, J=5.81 Hz, 2H) 6.45 (s, 1H) 7.47 (d, J=8.34 Hz, 2H) 7.83 (dd, J=8.21, 1.39 Hz, 1 H) 7.90-7.97 (m, 3H) 8.09 (d, J=8.34 Hz, 1H) 9.48 (t, J=5.81, 1H) 12.90 (br. s., 1H) 13.44 (s, 1H)

### Example 32

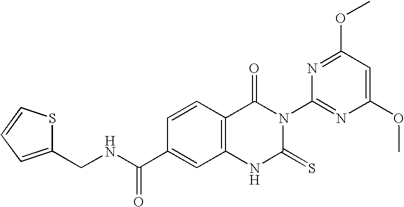

### 3-[4,6-Dimethoxy-2-pyrimidinyl]-4-oxo-N-(2-thienylmethyl)-2-thioxo-1,2,3,4-tetrahydro-7-quinazolinecarboxamide

To a stirred solution of 3-[4,6-dimethoxy-2-pyrimidinyl]-4-oxo-2-thioxo-1,2,3,4-tetrahydro-7-quinazolinecarboxylic acid (4a, 100 mg, 0.278 mmols, 1.0 eq.) in dry DMF (3 mL) were added 1-(2-thienyl)methylamine (32 mg, 0.278 mmol, 1.0 eq.) and EDC (54 mg, 0.278 mmol, 1.0 eq.). The solution stirred at rt overnight then diluted with water and extracted twice with ethyl acetate. The organic layer was washed once with brine and dried with anhydrous magnesium sulfate. The solvent was removed by rotary evaporation and the crude product was dissolved in DMSO (1 mL) then purified on HPLC under acidic conditions. Yield: 20 mg, 16%; LC/MS: M+1=456; ¹H NMR (400 MHz, MeOD-d₄) δ ppm: 3.95 (s, 6H), 4.77 (d, J=5.56 Hz, 2H), 6.28 (s, 1H), 6.97 (dd, J=5.18, 3.41 Hz, 1H), 7.07 (dd, J=3.41, 1.14 Hz, 1H), 7.31 (dd, J=5.31, 1.26 Hz, 1H), 7.70 (dd, J=8.34, 1.52 Hz, 1H), 7.75 (d, J=1.01 Hz, 1H), 8.14 (d, J=8.08 Hz, 1H), 9.32 (t, J=5.56 Hz, 1H).

### Example 33

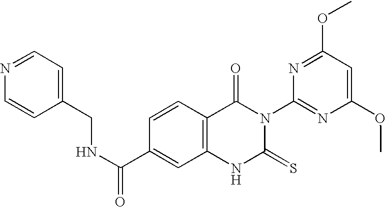

### 3-[4,6-Dimethoxy-2-pyrimidinyl]-4-oxo-N-(4-pyridinylmethyl)-2-thioxo-1,2,3,4-tetrahydro-7-quinazolinecarboxamide

To a vial of stirring PS-DCC (360 mg, 0.556 mmol, 3 eq.) in a 1:1 DCM and DMF solvent mixture (5 mL) was added 3-[4,6-dimethoxy-2-pyrimidinyl]-4-oxo-2-thioxo-1,2,3,4-tetrahydro-7-quinazolinecarboxylic acid (4a, 100 mg, 0.278 mmols, 1.5 eq.). The mixture was stirred for 30 min, followed by addition of 1-(4-pyridinyl)methylamine (20 mg, 0.185 mmol, 1.0 eq.). The mixture was stirred for 2 hours, the PS-DCC beads were filtered off and the solvent removed by rotary evaporation. The crude product was dissolved in DMSO (1 mL) and purified on HPLC under acidic conditions (5%-35% gradient in 10 minutes). Yield: 15.6 mg, 19%; LC/MS: M+1=451; ¹H NMR (400 MHz, MeOD-d₄) δ ppm: 3.96 (s, 6H), 4.88 (s, 2H), 6.30 (s, 1H), 7.80 (d, J=1.52 Hz, 1H), 7.82 (s, 1H), 8.03 (d, J=5.30 Hz, 2H), 8.19 (d, J=8.84 Hz, 1H), 8.79 (br. s., 2H).

### Example 34

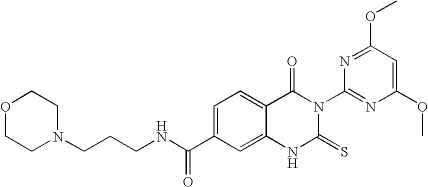

### 3-[4,6-Dimethoxy-2-pyrimidinyl]-N-[3-(4-morpholinyl)propyl]-4-oxo-2-thioxo-1,2,3,4-tetrahydro-7-quinazolinecarboxamide

To a vial of stirring PS-DCC (500 mg, 0.926 mmol, 5 eq.) in a 1:1 DCM and DMF solvent mixture (7 mL) was added 3-[4,6-dimethoxy-2-pyrimidinyl]-4-oxo-2-thioxo-1,2,3,4-tetrahydro-7-quinazolinecarboxylic acid (4a, 100 mg, 0.278 mmols, 1.5 eq.). The mixture was stirred for 2 hours, followed by addition of DMAP (50 mg, 0.278 mmol, 1.5 eq.) and 3-(4-morpholinyl)-1-propanamine (27 mg, 0.185 mmol, 1.0 eq.), in order. After the mixture was stirred for 3 additional hrs, the PS-DCC beads were filtered off and the solvent was removed by rotary evaporation. The crude product was dissolved in DMSO (1 mL) and was purified on HPLC under acidic conditions (5%-35% gradient in 10 minutes). Yield: 12.4 mg, 13%; LC/MS: M+1=487; ¹H NMR (400 MHz, MeOD-d₄) δ ppm: 2.04-2.15 (m, 2H), 3.17 (td, J=12.38, 3.54 Hz, 2H), 3.26 (dd, J=8.34, 1.77 Hz, 2H), 3.54 (t, 4H), 3.78 (t, J=11.87 Hz, 2H), 3.96 (s, 6H), 4.08 (dd, J=13.01, 1.89 Hz, 2H), 6.30 (s, 1H), 7.72 (dd, J=8.21, 1.64 Hz, 1H), 7.77 (d, J=1.01 Hz, 1H), 8.15 (d, J=8.34 Hz, 1H).

### Example 35

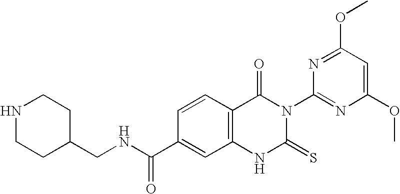

### 3-[4,6-Dimethoxy-2-pyrimidinyl]-4-oxo-N-(4-piperidinylmethyl)-2-thioxo-1,2,3,4-tetrahydro-7-quinazolinecarboxamide

To a stirred solution of 3-[4,6-dimethoxy-2-pyrimidinyl]-4-oxo-2-thioxo-1,2,3,4-tetrahydro-7-quinazolinecarboxylic acid (4a, 100 mg, 0.278 mmols, 1.0 eq.) in dry DMF (3 mL) were added 1,1-dimethylethyl 4-(aminomethyl)-1-piperidinecarboxylate (60 mg, 0.278 mmol, 1.0 eq.) and EDC (54 mg, 0.278 mmol, 1.0 eq.). The solution stirred at rt overnight then diluted with water and extracted twice with ethyl acetate. The solvent was removed by rotary evaporation and the crude material was dried overnight under vacuum. The crude product was then dissolved and stirred in a 1:1 DCM and TFA mixture (5 mL) for 1 hour. The solvent was then removed by rotary evaporation and the crude product in DMSO (1 mL) and was purified on HPLC under acidic conditions. Yield: 11 mg, 9%; LC/MS: M+1=457; ¹H NMR (400 MHz, MeOD-d₄) δ ppm 1.24 (t, J=7.20 Hz, 2H) 1.49 (dt, J=12.82, 3.19 Hz, 2H) 2.88-3.09 (m, 2H) 3.35-3.48 (m, 4H) 3.95 (s, 6H) 4.10 (q, J=7.07 Hz, 1H) 6.30 (s, 1H) 7.70 (dd, J=8.21, 1.64 Hz, 1H) 7.75 (d, J=1.01 Hz, 1H) 8.15 (d, J=8.08 Hz, 1H) 8.89 (t, J=5.81 Hz, 1H)

### Example 36

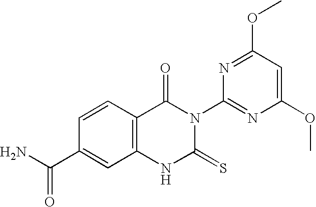

### 3-[4,6-Dimethoxy-2-pyrimidinyl]-4-oxo-2-thioxo-1,2,3,4-tetrahydro-7-quinazolinecarboxamide

To a stirred solution of 3-[4,6-Dimethoxy-2-pyrimidinyl]-4-oxo-2-thioxo-1,2,3,4-tetrahydro-7-quinazolinecarboxylic acid (4a, 100 mg, 0.278 mmols, 1.0 eq.) in dry DMF (3 mL) were added HATU (116 mg, 0.31 mmol, 1.1 eq.) and DIEA (0.053 mL, 0.31 mmol, 1.1 eq.). The solution was stirred at rt for 30 minutes then ammonia in methanol (7N, 0.04 mL, 0.278 mmol, 1.0 eq.) added and stirred at rt overnight. The solvent was removed by rotary evaporation and the crude product in DMSO (1 mL) purified on HPLC under acidic conditions. Yield: 39 mg, 39%; LC/MS: M+1=360; ¹H NMR (400 MHz, DMSO-d₆) δ ppm 3.88 (s, 6H) 6.45 (s, 1H) 7.74-7.80 (m, 2H) 7.88 (s, 1H) 8.04 (d, J=7.83 Hz, 1H) 8.28 (s, 1H) 13.44 (s, 1H)

### Example 37

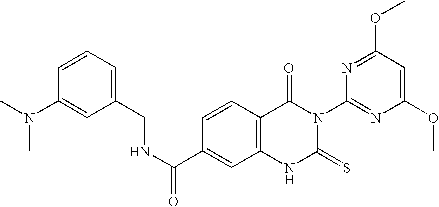

### 3-[4,6-Dimethoxy-2-pyrimidinyl]-N-{[3-(dimethylamino)-phenyl]methyl}-4-oxo-2-thioxo-1,2,3,4-tetrahydro-7-quinazolinecarboxamide

To a stirred solution of 3-[4,6-dimethoxy-2-pyrimidinyl]-4-oxo-2-thioxo-1,2,3,4-tetrahydro-7-quinazolinecarboxylic acid (4a, 100 mg, 0.278 mmols, 1.0 eq.) in dry DMF (3 mL) were added HATU (116 mg, 0.31 mmol, 1.1 eq.) and DIEA (0.053 mL, 0.31 mmol, 1.1 eq.). The solution was stirred at rt for 30 minutes then 3-(aminomethyl)-N,N-dimethylaniline (42 mg, 0.278 mmol, 1.0 eq.) added and stirred at rt overnight. The solvent was removed by rotary evaporation and the crude in DMSO (1 mL) purified on HPLC under acidic conditions. Yield: 72 mg, 53%; LC/MS: M+1=493; ¹H NMR (400 MHz, DMSO-d₆) δ ppm 2.95 (s, 6H) 3.89 (s, 6H) 4.46 (d, J=5.81 Hz, 2H) 6.45 (s, 1H) 6.75-6.95 (m, 3H) 7.23 (t, J=7.96 Hz, 1H) 7.80 (dd, J=8.34, 1.52 Hz, 1H) 7.90 (d, J=1.26 Hz, 1H) 8.08 (d, J=8.34 Hz, 1H) 9.36 (t, J=5.94 Hz, 1H) 13.43 (s, 1H)

### Example 38

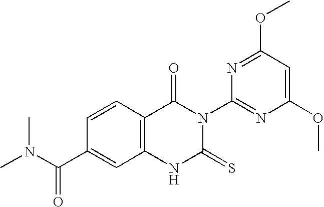

### 3-[4,6-Dimethoxy-2-pyrimidinyl]-N,N-dimethyl-4-oxo-2-thioxo-1,2,3,4-tetrahydro-7-quinazolinecarboxamide

To a stirred solution of 3-[4,6-dimethoxy-2-pyrimidinyl]-4-oxo-2-thioxo-1,2,3,4-tetrahydro-7-quinazolinecarboxylic acid (4a, 100 mg, 0.278 mmols, 1.0 eq.) in dry DMF (2 mL) were added HATU (116 mg, 0.31 mmol, 1.1 eq.) and DIEA (0.106 mL, 0.61 mmol, 2.2 eq.). The solution was stirred at room temperature for 30 minutes then N-methylmethylamine hydrochloride (23 mg, 0.278 mmol, 1.0 eq.) added and stirred at rt overnight. The solution was then diluted with water and extracted twice with ethyl acetate. The organic layer was washed once with brine, dried with magnesium sulfate and the solvent removed by rotary evaporation. The crude product in DMSO (1 mL) was purified on HPLC under acidic conditions. Yield: 34 mg, 32%; LC/MS: M+1=388; ¹H NMR (400 MHz, DMSO-d₆) δ ppm 2.90 (s, 3H) 3.03 (s, 4H) 3.89 (s, 6H) 6.45 (s, 0H) 7.34-7.40 (m, 2H) 8.03 (d, J=8.08 Hz, 1H) 13.36 (s, 1H)

### Example 39

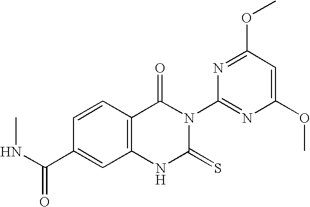

### 3-[4,6-Dimethoxy-2-pyrimidinyl]-N-methyl-4-oxo-2-thioxo-1,2,3,4-tetrahydro-7-quinazolinecarboxamide

To a stirred solution of 3-[4,6-dimethoxy-2-pyrimidinyl]-4-oxo-2-thioxo-1,2,3,4-tetrahydro-7-quinazolinecarboxylic acid (4a, 100 mg, 0.278 mmols, 1.0 eq.) in dry DMF (2 mL) were added HATU (116 mg, 0.31 mmol, 1.1 eq.) and DIEA (0.106 mL, 0.61 mmol, 2.2 eq.). The solution was stirred at room temperature for 30 minutes then methylamine in tetrahydrofuran (2M, 0.139 mL, 0.278 mmols, 1.0 eq.) added and stirred at rt overnight. The solvent was removed by rotary evaporation, and the crude product in DMSO (1 mL) purified on HPLC under acidic conditions. Yield: 40 mg, 39%; LC/MS: M+1=374; ¹H NMR (400 MHz, DMSO-d₆) δ ppm 2.82 (d, J=4.55 Hz, 3H) 3.88 (s, 6H) 6.45 (s, 1H) 7.73 (dd, J=8.21, 1.39 Hz, 1H) 7.86 (s, 1H) 8.06 (d, J=8.08 Hz, 1H) 8.79 (d, J=4.29, 1H) 13.43 (s, 1H)

### Example 40

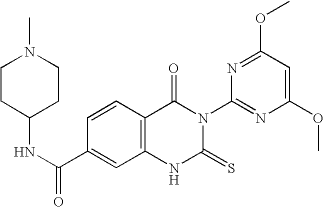

### 3-[4,6-Dimethoxy-2-pyrimidinyl]-N-(1-methyl-4-piperidinyl)-4-oxo-2-thioxo-1,2,3,4-tetrahydro-7-quinazolinecarboxamide

To a stirred solution of 3-[4,6-dimethoxy-2-pyrimidinyl]-4-oxo-2-thioxo-1,2,3,4-tetrahydro-7-quinazolinecarboxylic acid (4a, 100 mg, 0.278 mmols, 1.0 eq.) in dry DMF (2 mL) were added HATU (116 mg, 0.31 mmol, 1.1 eq.) and DIEA (0.106 mL, 0.61 mmol, 2.2 eq.). The solution was stirred at room temperature for 30 minutes then 1-methyl-4-piperidinamine (32 mg, 0.278 mmols, 1.0 eq.) added and stirred at rt overnight. The solvent was removed by rotary evaporation, and the crude product in DMSO (1 mL) purified on HPLC under acidic conditions. Yield: 72 mg, 57%; LC/MS: M+1=457; ¹H NMR (400 MHz, DMSO-d₆) δ ppm 1.68-1.85 (m, 2 H) 2.02-2.10 (m, 2H) 2.78 (d, J=4.80 Hz, 3H) 3.00-3.20 (m, 2H) 3.48 (d, J=12.63 Hz, 2H) 3.89 (s, 6H) 3.95-4.12 (m, 1H) 6.46 (s, 1H) 7.77 (dd, J=8.34, 1.52 Hz, 1H) 7.87 (s, 1H) 8.08 (d, J=8.08 Hz, 1H) 8.85 (d, J=7.33 Hz, 1H) 9.29 (br. s., 1H) 13.41 (s, 0H)

### Example 41

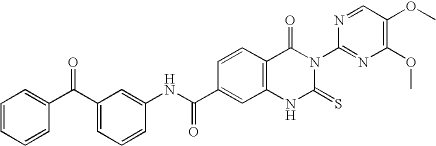

### N-[3-Benzoylphenyl]-3-[4,5-dimethoxy-2-pyrimidinyl]-4-oxo-2-thioxo-1,2,3,4-tetrahydro-7-quinazolinecarboxamide

To a stirred solution of 3-[4,5-dimethoxy-2-pyrimidinyl]-4-oxo-2-thioxo-1,2,3,4-tetrahydro-7-quinazolinecarboxylic acid (6a, 100 mg, 0.278 mmols, 1.0 eq.) in dry DMF (2 mL) were added HATU (116 mg, 0.31 mmol, 1.1 eq.) and DIEA (0.106 mL, 0.61 mmol, 2.2 eq.). The solution was stirred at room temperature for 30 minutes then 3-aminobenzophenone (55 mg, 0.278 mmols, 1.0 eq.) added and stirred at rt overnight. The solvent was removed by rotary evaporation, and the crude product in DMSO (1 mL) purified on HPLC under acidic conditions. Yield: not calculated; LC/MS: M+1=540; ¹H NMR (400 MHz, DMSO-d₆) δ ppm 3.92 (s, 3H) 3.97 (s, 3H) 7.49-7.56 (m, 1H) 7.60 (t, J=6.95 Hz, 3H) 7.71 (t, J=7.45 Hz, 1H) 7.79 (d, J=7.07 Hz, 2H) 7.91 (dd, J=8.21, 1.39 Hz, 1H) 7.94 (s, 1H) 8.13 (d, J=8.34 Hz, 2H) 8.21 (s, 1H) 8.40 (s, 1H) 10.86 (s, 1H) 13.44 (s, 1H)

### Example 42

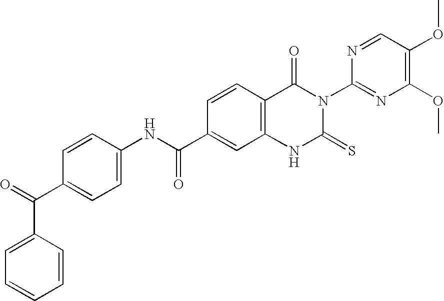

### N-[4-Benzoylphenyl]-3-[4,5-dimethoxy-2-pyrimidinyl]-4-oxo-2-thioxo-1,2,3,4-tetrahydro-7-quinazolinecarboxamide

To a stirred solution of 3-[4,5-dimethoxy-2-pyrimidinyl]-4-oxo-2-thioxo-1,2,3,4-tetrahydro-7-quinazolinecarboxylic acid (6a, 100 mg, 0.278 mmols, 1.0 eq.) in dry DMF (2 mL) were added HATU (116 mg, 0.31 mmol, 1.1 eq.) and DIEA (0.106 mL, 0.61 mmol, 2.2 eq.). The solution was stirred at room temperature for 30 minutes then 4-aminobenzophenone (55 mg, 0.278 mmols, 1.0 eq.) added and stirred at rt overnight. The solvent was removed by rotary evaporation, and the crude product in DMSO (1 mL) purified on HPLC under acidic conditions. Yield: not calculated; LC/MS: M+1=540; ¹H NMR (400 MHz, DMSO-d₆) δ ppm 3.92 (s, 3H) 3.98 (s, 3H) 7.58 (t, J=7.58 Hz, 2H) 7.69 (t, J=6.82 Hz, 1H) 7.74 (d, J=1.26 Hz, 2H) 7.83 (d, J=8.84 Hz, 2H) 7.92 (dd, J=8.34, 1.52 Hz, 1H) 7.95 (s, 1H) 8.00 (d, J=8.84 Hz, 2H) 8.15 (d, J=8.08 Hz, 1H) 8.40 (s, 1H) 10.99 (s, 1H) 13.45 (s, 1H)

### Example 43

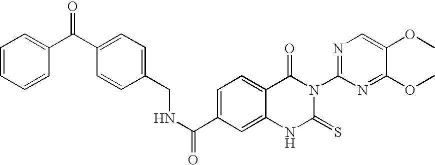

### N-[4-Benzoylbenzyl]-3-[4,5-dimethoxy-2-pyrimidinyl]-4-oxo-2-thioxo-1,2,3,4-tetrahydro-7-quinazolinecarboxamide

### 43a) 1-(4-Benzoylbenzyl)-3,5,7-triaza-1-azoniatricyclo[3.3.1.1^{3,7}]decane

Prepared using a literature procedure (Synthesis, (1979), 161). To a 100 mL round-bottomed flask was added 4-(benzoly)benzyl bromide (1 g, 3.6 mmol 1 eq), 1,3,5,7-tetraazatricyclo[3.3.1.1^{3,7}]decane (0.56 g, 4 mmol, 1.1 eq.) and sodium iodide (0.54 g, 3.6 mmol, 1 eq.) dissolved in 40 mL of ethanol. The solution was stirred overnight and the solid was filtered off and washed twice with cold ethanol to yield 1.22 g of white solid (100%). LC/MS: M+1=337; ¹H-NMR (400 MHz, DMSO-d₆) δ ppm: 7.87 (m, 4H), 7.72 (m, 3H), 7.62 (m, 2H), 5.15 (s, 6H), 4.62 (dd, 6H), 4.21 (s, 2H)

### 43b) 4-Benzoylbenzylamine

Prepared using a literature procedure (Synthesis, (1979), 161). To a 100 mL round bottomed flask was added 1-(4-benzoylbenzyl)-3,5,7-triaza-1-azoniatricyclo[3.3.1.1^{3,7}]decane (43a, 1 g, 2.9 mmol 1 eq), and 12N HCl (2 mL) in 30 mL of ethanol. The solution was heated 80° C. for 2 hours then evaporated, diluted with water and adjusted to pH 12 and extracted with ethyl acetate. The extracts were washed with brine, dried (MgSO4) and evaporated and the residue purified on HPLC under acidic condition (15-40% gradient). Yield=0.32 g (42%); LC/MS: M+1=212; ¹H-NMR (400 MHz, MeOD-d₄) δ ppm: 8.88 (d, 2H), 8.80 (d, 2H), 7.65 (m, 1H), 7.61 (d, 2H), 7.55 (m, 2H), 4.22 (s, 2H).

### 43c) N-(4-Benzoylbenzyl)-3-[4,5-dimethoxy-2-pyrimidinyl]-4-oxo-2-thioxo-1,2,3,4-tetrahydro-7-quinazolinecarboxamide

To a stirred solution of 3-[5,6-dimethoxy-2-pyrimidinyl]-4-oxo-2-thioxo-1,2,3,4-tetrahydro-7-quinazolinecarboxylic acid (6a, 60 mg, 0.17 mmol, 1 eq.) in dry DMF (1 mL) were added 4-benzoylbenzylamine (43b, 38 mg, 0.18 mmol, 1.1 eq.), DIEA (64 μL, 0.37 mmol, 2.2 eq.), and HATU (68 mg, 0.18 mmol, 1.1 eq.) in order. The mixture was stirred at rt overnight then diluted with ethyl acetate, washed with water, brine, dried (MgSO4) and evaporated and then purified on HPLC under acidic condition (15-40% gradient). Yield: 16 mg, 17%; LC/MS: M+1=554; ¹H-NMR (400 MHz, CDCl₃) δ ppm: 8.22 (s, 1H), 8.03 (d, 1H), 7.75-7.81 (m, 5H), 7.55-7.62 (m, 2H), 7.49-7.45 (m, 3H), 5.24 (s, 1H), 4.63 (s, 1H), 4.02 (s, 3H), 3.84 (s, 3H).

### Example 44

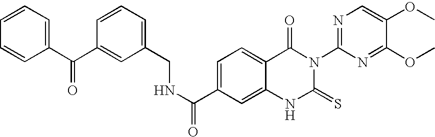

### N-(3-Benzoylbenzyl)-3-[4,5-dimethoxy-2-pyrimidinyl]-4-oxo-2-thioxo-1,2,3,4-tetrahydro-7-quinazolinecarboxamide

### 44a) 1-(3-Benzoylbenzyl)-3,5,7-triaza-1-azoniatricyclo[3.3.1.1^{3,7}]decane

Prepared using a literature procedure (Synthesis, (1979), 161). To a 100 mL round bottomed flask was added 3-benzoylbenzyl bromide (1 g, 3.6 mmol 1 eq), 1,3,5,7-tetraazatricyclo[3.3.1.1^{3,7}]decane (0.56 g, 4 mmol, 1.1 eq.) and sodium iodide (0.54 g, 3.6 mmol, 1 eq.) dissolved in 40 mL of ethanol. The solution was stirred overnight, the solid was filtered off and washed twice with cold ethanol to yield 1.22 g of white solid (100%); LC/MS: M+1=337; ¹H-NMR (400 MHz, DMSO-d₆) δ ppm: 7.87 (m, 4H), 7.72 (m, 3H), 7.62 (m, 2H), 5.15 (s, 6H), 4.61 (dd, 6H), 4.15 (s, 2H).

### 44b) 3-(Aminomethyl)benzophenone

Prepared using a literature procedure (Synthesis, (1979), 161). To a 100 mL round bottomed flask was added 1-(3-benzoylbenzyl)-3,5,7-triaza-1-azoniatricyclo[3.3.1.1^{3,7}]decane (44a, 1 g, 2.9 mmol 1 eq), and 12N HCl (2 mL) in 30 mL of ethanol. The solution was heated 80° C. for 2 hours then evaporated, diluted with water, adjusted to pH 12 and extracted with ethyl acetate. The extracts were washed with brine, dried (MgSO4), evaporated and the residue purified on HPLC under acidic condition (15-40% gradient). Yield=0.51 g (61%); LC/MS: M+1=212; ¹H-NMR (400 MHz, CDCl₃) δ ppm: 8.40 (s, 2H), 8.15 (s, 2H), 7.82 (s, 1H), 7.62 (m, 2H), 7.45 (m, 2H), 4.22 (s, 2H).

### 44c) N-(3-Benzoylbenzyl)-3-[4,5-dimethoxy-2-pyrimidinyl]-4-oxo-2-thioxo-1,2,3,4-tetrahydro-7-quinazolinecarboxamide

To a stirred solution of 3-[5,6-dimethoxy-2-pyrimidinyl]-4-oxo-2-thioxo-1,2,3,4-tetrahydro-7-quinazolinecarboxylic acid (6a, 75 mg, 0.21 mmol, 1 eq.) in dry DMF (1 mL) were added 3-benzoylbenzylamine (44b, 48 mg, 0.23 mmol, 1.1. eq.), DIEA (0.15 mL, 0.83 mmol, 4 eq.), and HATU (87 mg, 0.23 mmol, 1.1 eq.) in order. The mixture was stirred at rt overnight then diluted with ethyl acetate, washed with water, brine, dried (MgSO4) and evaporated. The residue in DMSO was purified on HPLC under acidic condition (15-40% gradient). Yield: 28 mg, 24%; LC/MS: M+1=554; ¹H-NMR (400 MHz, CDCl₃) δ ppm: 8.13 (br. s., 1H), 8.03 (d, 1H), 7.86 (m, 2H), 7.77 (d, 2H), 7.68 (m, 3H), 7.56 (t, 1H), 7.45 (q, 3H), 5.18 (s, 1H), 4.66 (s, 1H), 4.03 (s, 3H), 3.92 (s, 3H).

### Example 45

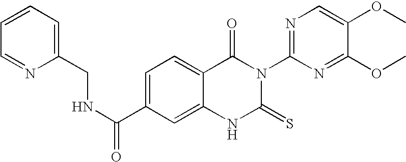

### 3-[4,5-Dimethoxy-2-pyrimidinyl]-4-oxo-N-(2-pyridinylmethyl)-2-thioxo-1,2,3,4-tetrahydro-7-quinazolinecarboxamide

To a stirred solution of 3-[5,6-dimethoxy-2-pyrimidinyl]-4-oxo-2-thioxo-1,2,3,4-tetrahydro-7-quinazolinecarboxylic acid (6a, 75 mg, 0.21 mmol, 1 eq.) in dry DMF (1 mL) were added (2-pyridinylmethyl)amine (25 mg, 0.23 mmol, 1.1 eq.), DIEA (0.1 mL, 0.46 mmol, 2.2 eq.), and HATU (87 mg, 0.23 mmol, 1.1 eq.) in order. The mixture was stirred at rt overnight then the solution was diluted with ethyl acetate, washed with water, brine, dried (MgSO4) and evaporated. The residue in DMSO was purified on HPLC under acidic condition (15-40% gradient). Yield: 10.5 mg, 11%; LC/MS: M+1=451; ¹H-NMR (400 MHz, DMSO-d₆) δ ppm: 13.40 (br. s., 1H), 9.50 (t, 1H), 8.60 (d, 1H), 8.39 (s, 1H), 8.10 (d, 1H), 7.90 (s, 2H), 7.85 (d, 1H), 7.49 (d, 1H), 7.41 (m, 1H), 4.64 (s, 2H), 3.97 (s, 3H), 3.91 (s, 3H).

### Example 46

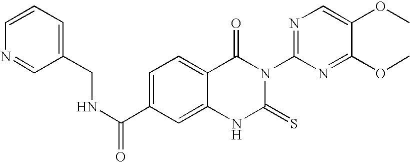

### 3-[4,5-Dimethoxy-2-pyrimidinyl]-4-oxo-N-(3-pyridinylmethyl)-2-thioxo-1,2,3,4-tetrahydro-7-quinazolinecarboxamide

To a stirred solution of 3-[5,6-dimethoxy-2-pyrimidinyl]-4-oxo-2-thioxo-1,2,3,4-tetrahydro-7-quinazolinecarboxylic acid (6a, 75 mg, 0.21 mmol, 1 eq.) in dry DMF (1 mL) were added (3-pyridinylmethyl)amine (25 mg, 0.23 mmol, 1.1 eq.), DIEA (0.1 mL, 0.46 mmol, 2.2 eq.), and HATU (87 mg, 0.23 mmol, 1.1 eq.) in order. The mixture was stirred at rt overnight, diluted with ethyl acetate, washed with water, brine, dried (MgSO4) and evaporated. The reiduein DMSO was purified on HPLC under acidic condition (15-40% gradient). Yield: 48 mg, 51%; LC/MS: M+1=451; ¹H-NMR (400 MHz, DMSO-d₆) δ ppm: 13.40 (br. s., 1H), 9.51 (t, 1H), 8.75 (s, 1H), 8.65 (d, 1H), 8.39 (s, 1H), 8.10 (m, 2H), 7.91 (s, 1H), 7.81 (d, 1H), 7.71 (m, 1H), 4.61 (s, 2H), 3.97 (s, 3H), 3.91 (s, 3H).

### Example 47

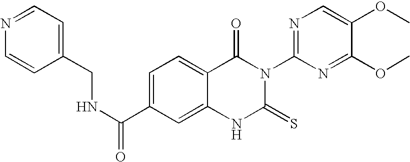

### 3-[4,5-Dimethoxy-2-pyrimidinyl]-4-oxo-N-(4-pyridinylmethyl)-2-thioxo-1,2,3,4-tetrahydro-7-quinazolinecarboxamide

To a stirred solution of 3-[5,6-dimethoxy-2-pyrimidinyl]-4-oxo-2-thioxo-1,2,3,4-tetrahydro-7-quinazolinecarboxylic acid (6a, 75 mg, 0.21 mmol, 1 eq.) in dry DMF (1 mL) were added (4-pyridinylmethyl)amine (25 mg, 0.23 mmol, 1.1 eq.), DIEA (0.1 mL, 0.46 mmol, 2.2 eq.), and HATU (87 mg, 0.23 mmol, 1.1 eq.) in order. The mixture was stirred at room temperature overnight, diluted with ethyl acetate, washed with water, brine and dried (MgSO4) and evaporated. The residue in DMSO was purified on HPLC under acidic condition (15-40% gradient). Yield: 43 mg, 46%; LC/MS: M+1=451; ¹H-NMR (400 MHz, DMSO-d₆) δ ppm: 13.42 (br. s., 1H), 9.62 (t, 1H), 8.75 (s, 2H), 8.39 (s, 1H), 8.12 (d, 1H), 7.92 (s, 1H), 7.85 (d, 1H), 7.76 (m, 2H), 4.69 (s, 2H), 3.97 (s, 3H), 3.91 (s, 3H).

### Example 48

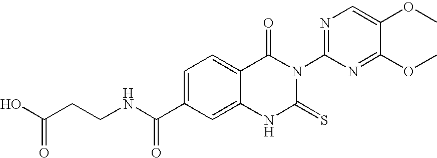

### N-({3-[4,5-Dimethoxy-2-pyrimidinyl]-4-oxo-2-thioxo-1,2,3,4-tetrahydro-7-quinazolinyl}carbonyl)-glycine

To a stirred solution of 3-[5,6-dimethoxy-2-pyrimidinyl]-4-oxo-2-thioxo-1,2,3,4-tetrahydro-7-quinazolinecarboxylic acid (6a, 60 mg, 0.17 mmol, 1 eq.) in dry DMF (1 mL) were added t-butyl glycinate (24 mg, 0.18 mmol, 1.1 eq.), DIEA (64 □L, 0.37 mmol, 2.2 eq.), and HATU (68 mg, 0.18 mmol, 1.1 eq.) in order. The mixture was stirred at rt overnight, diluted with ethyl acetate, washed with water, brine, dried (MgSO4) and evaporated. The residue was treated with 50% TFA/DCM (1 hour, rt) evaporated and residue in DMSO purified on HPLC under acidic condition (15-45% gradient). Yield: 28 mg, 41%; LC/MS: M+1=418; ¹H-NMR (400 MHz, DMSO-d₆) δ ppm: 13.41 (br. s., 1H), 9.20 (t, 1H), 8.39 (s, 1H), 8.10 (d, 1H), 7.90 (s, 1H), 7.77 (d, 1H), 3.97-3.91 (m, 8H).

### Example 49

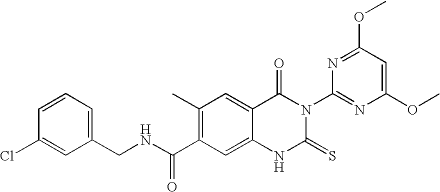

### N-(3-Chlorobenzyl)-3-[4,6-dimethoxy-2-pyrimidinyl]-6-methyl-4-oxo-2-thioxo-1,2,3,4-tetrahydro-7-quinazolinecarboxamide

### 49a) dimethyl 2-amino-5-methyl-1,4-benzenedicarboxylate

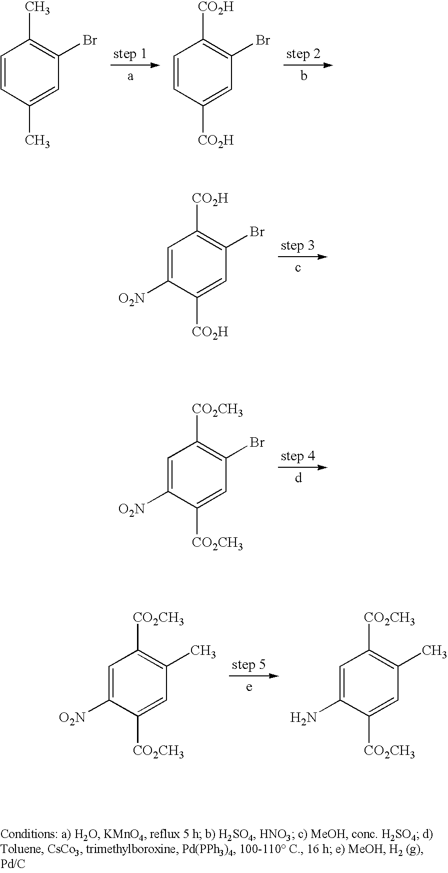

Step 1: A mixture of 2-bromo-p-xylene (18.5 g, 100 mmole) and KMnO₄(15.8 g; 100 mmole) in water (225 ml) was refluxed for 2 h under stirring. After the disappearance of KMnO₄color, TLC showed the presence of starting material. Additional KMnO₄(15.8 g; 100 mmole) was added and refluxing continued for 2 h. TLC showed the presence of starting material, another lot of KMnO₄(15.8 g; 100 mmole) was added and refluxing continued for 2 h. TLC showed the presence of starting material; however, the reaction was worked up. The mixture was cooled to ambient temperature and filtered. The filtrate was extracted with ethyl acetate (2×25 ml). The organic layer was dried and evaporated to recover 6.15 g (33%) of the starting material. The aqueous filtrate was concentrated to half volume on a rotavap. The concentrated aqueous mixture was cooled to 0-5° C. and acidified to pH 2 with conc. HCl. The precipitated solid was filtered and washed with water to yield 11.39 g (47%) of 2-bromo terephthalic acid as a colorless solid. ¹H-NMR in CD₃OD-d₄δ ppm: 7.86 (1H, d, J=7.8 Hz, Ar—H), 8.05 (1H, dd, J=8.4 Hz & 1.6 Hz, Ar—H), 8.28 (1H, d, J=1.6 Hz, Ar—H).

Step 2: 2-Bromo terephthalic acid (13.8 g, 56.3 mmole) was slowly added under stirring to conc. H₂SO₄(78 ml) at 0-5° C. over 5 minutes. To the resulting mixture was added 1:1 mixture of conc. H₂SO₄and conc. HNO₃(15 ml) dropwise over 20 min. at 0-5° C. The mixture was heated to 100° C. for 2 h. After cooling and stirring for 18 h at RT, mixture was poured into 100 g of ice-water. The resulting colorless solid was filtered and dried. The solid was recrystallized from ethanol to get 10.5 g (64%) of 2-bromo-5-nitroterephthalic acid; ¹H-NMR in CD₃OD-d₄δ ppm: 8.14 (1H, s, Ar—H), 8.34 (1H, s, Ar—H).

Step 3: To a suspension of 2-bromo-5-nitroterephthalic acid (10.5 g; 36.2 mmole) in methanol (200 ml) was added dropwise conc. H₂SO₄(5 ml) at ambient temperature. The mixture was refluxed for 18 h. TLC showed the disappearance of starting material and formation of non-polar product along with small amount of monoester. Methanol was distilled out on a rotavap and the resulting solid was stirred with water (25 ml), filtered and washed with water. The wet solid was dissolved in ethyl acetate (100 ml) and washed with aq. sat. NaHCO₃solution (25 ml). The ethyl acetate layer was dried and evaporated to give 8 g (69%) of dimethyl 2-bromo-5-nitroterephthalate as a colorless solid. ¹H-NMR in CD₃OD-d₄δ ppm: 3.96 (3H, s, OCH₃), 4.01 (3H, s, OCH₃), 8.16 (1H, s, Ar—H), 8.41 (1H, s, Ar—H).

Step 4: To a solution of dimethyl 2-bromo-5-nitroterephthalate (10.7 g; 33.5 mmole) in toluene (50 ml), cesium carbonate (32.7 g; 100.5 mmole), tetrakis-(triphenylphosphine)palladium (3.8 g; 3.3 mmole) and trimethylboroxine (4.2 g; 33.5 mmole) were added sequentially at ambient temperature under nitrogen. The mixture was heated to 100-110° C. for 8 h under nitrogen. TLC of the reaction mixture showed the presence of starting material, the mixture was cooled to room temperature and another lot of trimethylboroxine (4.2 g; 33.5 mmole) was added. The mixture was again heated to 100-110° C. for 8 h under nitrogen. Mixture was cooled to ambient temperature and filtered. The filtrate was concentrated on a rotavap and resulting brown semi-solid was purified by column chromatography over silica gel (5% ethyl acetate in hexane) to yield 6 g (70%) of dimethyl 2-methyl-5-nitroterephthalate as colorless solid. ¹H-NMR, DMSO-d₆δ ppm: 2.63 (3H, s, CH₃), 3.87 (3H, s, OCH₃), 3.88 (3H, s, OCH₃), 7.82 (1H, s, Ar—H), 8.4 (1H, s, Ar—H).

Step 5: A mixture of dimethyl 2-methyl-5-nitroterephthalate (5.7 g; 22.4 mmoles), 5% Pd/C (0.7 g) in methanol (300 ml) was hydrogenated under a hydrogen pressure of 40 psi for 1 h in a Parr hydrogenator. The mixture was filtered under nitrogen and the filtrate was concentrated to give 4.7 g (89%) of dimethyl 2-amino-5-methylterephthalate as yellow solid. ¹H-NMR in DMSO-d₆ δ ppm: 2.29 (3H, s, CH₃), 3.78 (3H, s, OCH₃), 3.79 (3H, s, OCH₃), 6.59 (2H, s, NH₂), 7.24 (1H, s, Ar—H), 7.56 (1H, s, Ar—H).

### 49b) Dimethyl 2-isothiocyanato-5-methyl-1,4-benzenedicarboxylate

To a biphasic mixture of dimethyl 2-amino-5-methyl-1,4-benzenedicarboxylate (1.00 g, 4.48 mmol) in chloroform (12 mL) and saturated aqueous sodium bicarbonate (12 mL), thiophosgene (618 mg, 5.38 mmol) was added dropwise. The mixture was stirred vigorously at rt overnight. The phases were separated and the aqueous layer was extracted with DCM. The combined organic layer was dried (MgSO₄), filtered, and concentrated to furnish the title compound (49b, 1.21 g, 4.56 mmol) which was used without purification.

### 49c) N-(3-Chlorobenzyl)-3-[4,6-dimethoxy-2-pyrimidinyl]-6-methyl-4-oxo-2-thioxo-1,2,3,4-tetrahydro-7-quinazolinecarboxamide

A vial containing dimethyl 2-isothiocyanato-5-methyl-1,4-benzenedicarboxylate (49b, 100 mg, 0.38 mmol) and 4,6-dimethoxy-2-pyrimidinamine (59 mg, 0.38 mmol) in DMF (0.75 mL) was stirred at 80° C. overnight. 10% sodium hydroxide (0.5 mL) was added dropwise and the mixture was stirred at rt for 2 hours. The mixture was acidified with 6N hydrochloric acid to pH ˜6 and the reaction mixture was concentrated. Water was added to the residue, the mixture was centrifuged, and the supernatent was removed for a total of 3 cycles. The residue was dried in vacuo, dissolved in DMF (1.5 mL), then DIEA (73 uL, 0.42 mmol) and HATU (160 mg, 0.42 mmol) were added. The mixture was stirred at rt for 90 min, then 3-chlorobenzylamine (59 mg, 0.42 mmol) was added. The mixture was stirred at rt overnight, then purified by reversed-phase HPLC to provide the product (17.1 mg) as a TFA salt. ¹H NMR (400 MHz, DMSO-d₆) δ ppm: 2.36 (s, 3H), 3.89 (s, 6H), 4.49 (d, J=6.06 Hz, 2H), 6.45 (s, 1H), 7.32-7.45 (m, 5H), 7.87 (s, 1H), 9.21 (t, J=5.94 Hz, 1H), 13.32 (s, 1H); MS m/z (MH⁺) 498.1

### Example 50

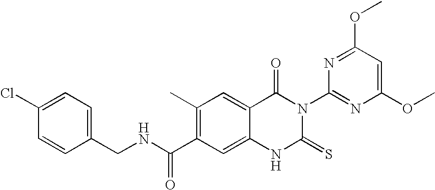

### N-(4-Chlorobenzyl)-3-[4,6-dimethoxy-2-pyrimidinyl]-6-methyl-4-oxo-2-thioxo-1,2,3,4-tetrahydro-7-quinazolinecarboxamide

A vial containing dimethyl 2-isothiocyanato-5-methyl-1,4-benzenedicarboxylate (49b, 100 mg, 0.38 mmol) and 4,6-dimethoxy-2-pyrimidinamine (59 mg, 0.38 mmol) in DMF (0.75 mL) was stirred at 80° C. overnight. 10% sodium hydroxide (0.5 mL) was added dropwise and the mixture was stirred at rt for 2 hours. The mixture was acidified with 6N hydrochloric acid to pH ˜6 and the reaction mixture was concentrated. Water was added to the residue, the mixture was centrifuged, and the supernatent was removed for a total of 3 cycles. The residue was dried in vacuo, dissolved in DMF (1.5 mL), then DIEA (73 uL, 0.42 mmol) and HATU (160 mg, 0.42 mmol) were added. The mixture was stirred at rt for 90 minutes, then 4-chlorobenzylamine (59 mg, 0.42 mmol) was added. The mixture was stirred at rt overnight, then purified by reversed-phase HPLC to provide the product (26.3 mg) as a TFA salt. ¹H NMR (400 MHz, DMSO-d₆) δ ppm: 2.35 (s, 3H), 3.89 (s, 6H), 4.47 (d, J=6.06 Hz, 2H), 6.45 (s, 1H), 7.38-7.47 (m, 5H), 7.86 (s, 1H), 9.19 (t, J=5.94 Hz, 1H), 13.31 (s, 1H); MS m/z (MH⁺) 498.1

### Example 51

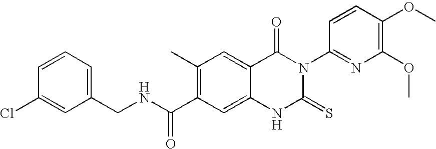

### N-(3-Chlorobenzyl)-3-[5,6-dimethoxy-2-pyridinyl]-6-methyl-4-oxo-2-thioxo-1,2,3,4-tetrahydro-7-quinazolinecarboxamide

A vial containing dimethyl 2-isothiocyanato-5-methyl-1,4-benzenedicarboxylate (49b, 100 mg, 0.38 mmol) and 4,6-dimethoxy-2-pyridinamine (59 mg, 0.38 mmol) in DMF (0.75 mL) was stirred at rt overnight. 10% sodium hydroxide (0.5 mL) was added dropwise and the mixture was stirred at rt for 2 hr. The mixture was acidified with 6N hydrochloric acid to pH ˜6 and the reaction mixture was concentrated. Water was added to the residue, the mixture was centrifuged, and the supernatent was removed for a total of 3 cycles. The residue was dried in vacuo, dissolved in DMF (1.5 mL), then DIEA (73 uL, 0.42 mmol) and HATU (160 mg, 0.42 mmol) were added. The mixture was stirred at rt for 90 minutes, then 3-chlorobenzylamine (59 mg, 0.42 mmol) was added. The mixture was stirred at rt overnight, then purified by reversed-phase HPLC to provide the product (59.7 mg) as a TFA salt. ¹H NMR (400 MHz, DMSO-d₆) δ ppm: 2.34 (s, 3H), 3.78 (s, 3H), 3.86 (s, 3H), 4.48 (d, 2H), 7.01 (s, 1H), 7.32-7.47 (m, 6H), 7.83 (s, 1H), 9.16 (t, 1H), 13.11 (s, 1H); MS m/z (MH⁺) 497.3

### Example 52

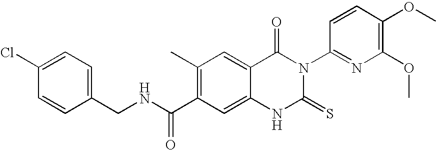

### N-(4-Chlorobenzyl)-3-[5,6-dimethoxy-2-pyridinyl]-6-methyl-4-oxo-2-thioxo-1,2,3,4-tetrahydro-7-quinazolinecarboxamide

A vial containing dimethyl 2-isothiocyanato-5-methyl-1,4-benzenedicarboxylate (49b, 100 mg, 0.38 mmol) and 4,6-bis(methyloxy)-2-pyridinamine (59 mg, 0.38 mmol) in DMF (0.75 mL) was stirred at rt overnight. 10% sodium hydroxide (0.5 mL) was added dropwise and the mixture was stirred at rt for 2 hr. The mixture was acidified with 6N hydrochloric acid to pH ˜6 and the reaction mixture was concentrated. Water was added to the residue, the mixture was centrifuged, and the supernatent was removed for a total of 3 cycles. The residue was dried in vacuo, dissolved in DMF (1.5 mL), then DIEA (73 uL, 0.42 mmol) and HATU (160 mg, 0.42 mmol) were added. The mixture was stirred at rt for 90 min, then 4-chlorobenzylamine (59 mg, 0.42 mmol) was added. The mixture was stirred at rt overnight, then purified by reversed-phase HPLC to provide the product (85 mg) as a TFA salt. ¹H NMR (400 MHz, DMSO-d₆) δ ppm: 2.34 (s, 3H), 3.78 (s, 3H), 3.86 (s, 3H), 4.48 (d, 2H), 7.01 (s, 1H), 7.32-7.47 (m, 6H), 7.83 (s, 1H), 9.16 (t, 1H), 13.11 (s, 1H); MS m/z (MH⁺) 497.1

### Example 53

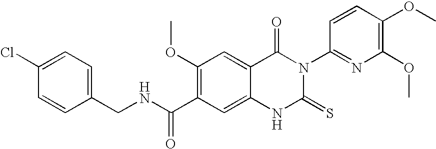

### N-(4-Chlorobenzyl)-3-[5,6-dimethoxy-2-pyridinyl]-6-(methoxy)-4-oxo-2-thioxo-1,2,3,4-tetrahydro-7-quinazolinecarboxamide

### 53a) dimethyl 2-amino-5-(methyloxy)-1,4-benzenedicarboxylate

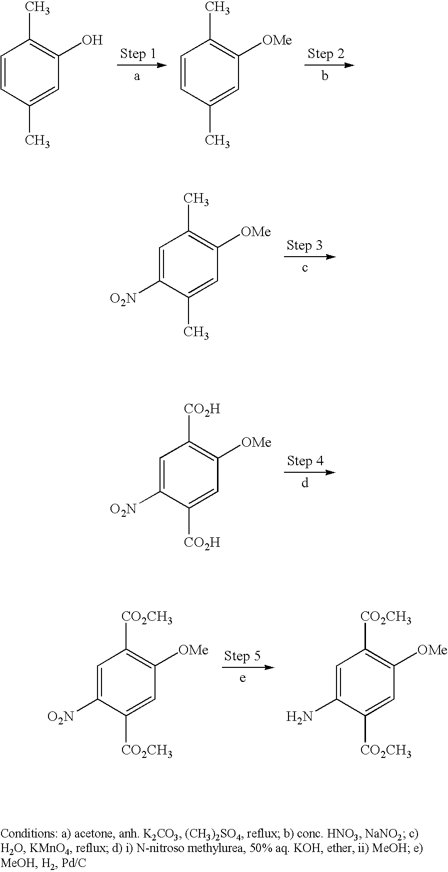

Step 1: To a mixture of 2,5-dimethyl phenol (50 g, 410 mmole), and potassium carbonate (68 g; 490 mmole) in acetone (600 ml), dimethyl sulfate (31.02 g, 246 mmole) was added at ambient temperature. The mixture was reflux for 9 h after which TLC revealed presence of the starting material. Additional dimethyl sulfate (31.02 g, 246 mmole) was added and reaction mixture was refluxed for another 9 h. The reaction mixture was filtered and acetone was removed on a rotavap. The resulting oil was stirred with 20% NaOH (100 ml) for 10 minutes. The organic layer was washed with water (2×500 ml) till the aqueous layer was neutral. The organic layer was dried over sodium sulfate and concentrated under vacuum to yield 45.5 g of 2-methoxy-1,4-dimethylbenzene (81%). ¹H NMR in CDCl₃δ ppm: 2.2 (3H, s, CH₃), 2.34 (3H, s, CH₃), 3.82 (3H, s, OCH₃), 6.65 (1H, s, Ar—H), 6.7 (1H, d, J=7.2 Hz, Ar—H), 7.03 (1H, d, J=7.2 Hz, Ar—H).

Step 2: To an ice-cold conc. nitric acid (500 ml) was added slowly under stirring over a period of 20 minutes 2-methoxy-1,4-dimethylbenzene (45 g, 330 mmole). To this cold reaction mixture, sodium nitrite (67.5 g, 990 mmole) was added slowly in lots over a period of 1 h while maintaining the temperature below 2° C. The reaction mixture was stirred between 0-5° C. for 5 h. The reaction mass was poured over ice-cold water (2000 ml) and the precipitated solid was filtered, washed with cold water (200 ml) and dried. The crude solid was crystallized from ethanol and water (7:1) to yield 28 g of 2-methoxy-5-nitro-1,4-dimethylbenzene (46%). ¹H NMR in CDCl₃δ ppm: 2.21 (3H, s, CH₃), 2.63 (3H, s, CH₃), 3.89 (3H, s, OCH₃), 6.64 (1H, s, Ar—H), 7.9 (1H, s, Ar—H).

Step 3: A mixture of 2-methoxy-5-nitro-1,4-dimethylbenzene (28 g, 153 mmole) and KMnO₄(79 g; 500 mmole) in water (1500 ml) was reflux for 5 h under stirring. After the disappearance of KMnO₄color, TLC showed the presence of starting material. Additional KMnO₄ (79 g; 500 mmole) was added and refluxing continued for 5 h. TLC showed the presence of starting material, another lot of KMnO₄ (50.5 g; 320 mmole) was added and refluxing continued for 5 h. The reaction mixture was cooled to room temperature and filtered. The filtrate was concentrated to approx. 300 ml on a rotavap. The concentrated aqueous mixture was cooled to 0-5° C. and acidified to pH 2 with conc. HCl. The precipitated solid was filtered and washed with water to yield 18.5 g of 2-methoxy-5-nitroterephthalic acid as a colorless solid after drying (50%). ¹H-NMR in DMSO-δ₆δ ppm: 3.98 (3H, s, OCH₃), 7.41 (1H, s, Ar—H), 8.3 (1H, s, ArH).

Step 4: To a solution of 2-methoxy-5-nitroterephthalic acid (15 g, 61 mmole) in methanol (200 ml), an ethereal solution of diazomethane (1000 ml) [prepared from 50% aq. KOH (100 ml) and N-nitroso methylurea (60 g, 580 mmole)] was added maintaining temperature between 0-5° C. over a period of 30 minutes. The reaction mixture was stirred between 0-5° C. for 1 h and then allowed to come to room temperature. Excess of diazomethane was quenched by acetic acid. The reaction mixture was dried over Na₂SO₄and concentrated to yield 15.5 g of dimethyl 2-methoxy-5-nitroterephthalate (94%) as colorless solid. ¹H-NMR in CDCl₃δ ppm: 3.92 (3H, s, OCH₃), 3.95 (3H, s, COOCH₃), 4.02 (3H, s, COOCH₃), 7.11 (1H, s, Ar—H), 8.53 (1H, s, Ar—H).

Step 5: A mixture of dimethyl 2-methoxy-5-nitroterephthalate (15.5 g; 57 mmoles), 5% Pd/C (1 g) in methanol (160 ml) was hydrogenated under hydrogen pressure of 50 psi for 1 h in a Parr hydrogenator. Mixture was filtered under nitrogen and filtrate was concentrated to get 13.5 g (99%) of dimethyl 2-amino-5-methoxyterephthalate as yellow solid. ¹H-NMR in CDCl₃δ ppm: 3.38 (3H, s, OCH₃), 3.88 (3H, s, COOCH₃), 3.89 (3H, s, COOCH₃), 5.42 (2H, bs, NH₂), 7.07 (1H, s, Ar—H), 7.41 (1H, s, Ar—H).

### 53b) Dimethyl 2-isothiocyanato-5-methoxy-1,4-benzenedicarboxylate

To a biphasic mixture of dimethyl 2-amino-5-methoxy-1,4-benzenedicarboxylate (53a, 270 mg, 1.13 mmol) in chloroform (5 mL) and saturated aqueous sodium bicarbonate (5 mL), thiophosgene (194 mg, 1.69 mmol) was added dropwise and the mixture stirred vigorously at rt overnight. The phases were separated and the aqueous layer was extracted with DCM. The combined organic layer was dried (MgSO₄), filtered, and concentrated to furnish the title compound (302 mg, 1.07 mmol) which was used without purification.

### 53c) 3-[5,6-Dimethoxy-2-pyridinyl]-6-methoxy-4-oxo-2-thioxo-1,2,3,4-tetrahydro-7-quinazolinecarboxylic acid

To a solution of dimethyl 2-isothiocyanato-5-methoxy-1,4-benzenedicarboxylate (53b, 100 mg, 0.36 mmol) in DMF (0.6 mL) was added 4,6-dimethoxy-2-pyridinamine (55 mg, 0.36 mmol). The mixture was stirred at 60° C. overnight, then 10% sodium hydroxide (0.5 mL) was added and the mixture was stirred at rt for 2 hours. The mixture was acidified with 6N hydrochloric acid to pH ˜4 and concentrated to a residue. The residue was washed with water, centrifuged, and the supernatent was discarded. The residue was dissolved in DMSO, filtered, and purified by reversed-phase HPLC to furnish the title compound (41 mg). MS m/z (MH⁺) 390.0

### 53d) N-(4-Chlorobenzyl)-3-[5,6-dimethoxy-2-pyridinyl]-6-methoxy-4-oxo-2-thioxo-1,2,3,4-tetrahydro-7-quinazolinecarboxamide

To a solution of 3-[5,6-dimethoxy-2-pyridinyl]-6-methoxy-4-oxo-2-thioxo-1,2,3,4-tetrahydro-7-quinazolinecarboxylic acid (53c, 40 mg, 0.10 mmol) in DMF (1 mL) was added DIEA (14 mg, 0.11 mmol) and HATU (42 mg, 0.11 mmol). After stirring for 15 min, 4-chlorobenzylamine (14 mg, 0.10 mmol) was added and the mixture was stirred at rt overnight, then purified by reversed-phase HPLC to provide the product (29.2 mg) as a TFA salt. ¹H NMR (400 MHz, DMSO-d₆) δ ppm: 3.78 (s, 3H), 3.86 (s, 3H), 3.95 (s, 3H), 4.49 (d, J=6.06 Hz, 2H), 7.01 (d, J=8.08 Hz, 1H), 7.36-7.48 (m, 5H), 7.50 (s, 1H), 7.72 (s, 1H), 9.04 (t, J=6.06 Hz, 1H), 13.14 (s, 1H); MS m/z (MH⁺) 513.2

### Example 54

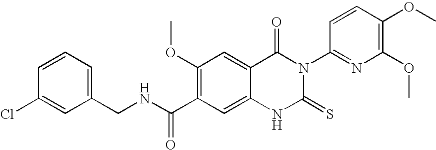

### N-(3-Chlorobenzyl)-3-[5,6-dimethoxy-2-pyridinyl]-6-methoxy-4-oxo-2-thioxo-1,2,3,4-tetrahydro-7-quinazolinecarboxamide

To a solution of 3-[5,6-dimethoxy-2-pyridinyl]-6-methoxy-4-oxo-2-thioxo-1,2,3,4-tetrahydro-7-quinazolinecarboxylic acid (53c, 26 mg, 0.07 mmol) in DMF (0.65 mL) was added DIEA (9 mg, 0.07 mmol) and HATU (28 mg, 0.07 mmol). After stirring for 15 min, 3-chlorobenzylamine (9 mg, 0.07 mmol) was added and the mixture was stirred at rt overnight, then purified by reversed-phase HPLC to provide the product (22.1 mg) as a TFA salt. ¹H NMR (400 MHz, DMSO-d₆) δ ppm: 3.78 (s, 3H), 3.86 (s, 3H), 3.96 (s, 3H), 4.52 (d, J=6.06 Hz, 2H), 7.02 (d, J=8.08 Hz, 1H), 7.31-7.36 (m, 2H), 7.38-7.48 (m, 3H), 7.51 (s, 1H), 7.69 (s, 1H), 9.07 (t, J=6.19 Hz, 1H), 13.15 (s, 1H); MS m/z (MH⁺) 513.2

### Example 55

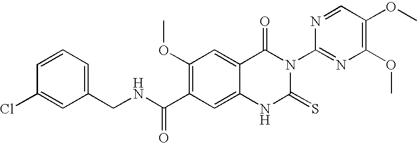

### N-(3-Chlorobenzyl)-3-[4,5-dimethoxy-2-pyrimidinyl]-6-methoxy-4-oxo-2-thioxo-1,2,3,4-tetrahydro-7-quinazolinecarboxamide

### 55a) 3-[4,5-Dimethoxy-2-pyrimidinyl]-6-methoxy-4-oxo-2-thioxo-1,2,3,4-tetrahydro-7-quinazolinecarboxylic acid

To a solution of dimethyl 2-isothiocyanato-5-methoxy-1,4-benzenedicarboxylate (53b, 86 mg, 0.30 mmol) in DMF (1.5 mL) was added 4,5-dimethoxy-2-pyrimidinamine (47 mg, 0.30 mmol). The mixture was stirred at 60° C. overnight, then 10% sodium hydroxide (0.3 mL) was added and the mixture was stirred at rt for 2 hours. The mixture was acidified with 6N hydrochloric acid to pH ˜4 and concentrated to a residue. The residue was washed with water, centrifuged, and the supernatent was discarded. The residue was dissolved in DMSO, filtered, and purified by reversed-phase HPLC to furnish the title compound (36 mg). MS m/z (MH⁺) 390.9

### 55b N-(3-Chlorobenzyl))-3-[4,5-dimethoxy-2-pyrimidinyl]-6-methoxy)-4-oxo-2-thioxo-1,2,3,4-tetrahydro-7-quinazolinecarboxamide

To a solution of 3-[4,5-dimethoxy-2-pyrimidinyl]-6-methoxy-4-oxo-2-thioxo-1,2,3,4-tetrahydro-7-quinazolinecarboxylic acid (55a, 18 mg, 0.05 mmol) in DMF (0.5 mL) was added DIEA (6 mg, 0.05 mmol) and HATU (19 mg, 0.05 mmol). After stirring for 60 min, 3-chlorobenzylamine (7 mg, 0.05 mmol) was added and the mixture was stirred at rt overnight, then purified by reversed-phase HPLC to provide the product (7 mg) as a TFA salt. ¹H NMR (400 MHz, DMSO-d₆) δ ppm: 3.91 (s, 3H), 3.96 (s, 3H), 3.97 (s, 3H), 4.51 (d, J=6.06 Hz, 2H), 7.33 (t, J=6.95 Hz, 2H), 7.37-7.45 (m, 2H), 7.52 (s, 1H), 7.68 (s, 1H), 8.39 (s, 1H), 9.09 (t, J=6.06 Hz, 1H), 13.29 (s, 1H) MS m/z (MH⁺) 514.2

### Example 56

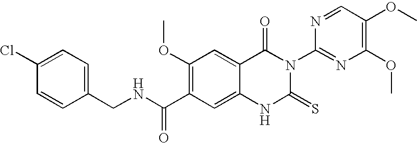

### N-(4-Chlorobenzyl)-3-[4,5-dimethoxy-2-pyrimidinyl]-6-methoxy-4-oxo-2-thioxo-1,2,3,4-tetrahydro-7-quinazolinecarboxamide

To a solution of 3-[4,5-dimethoxy-2-pyrimidinyl]-6-methoxy-4-oxo-2-thioxo-1,2,3,4-tetrahydro-7-quinazolinecarboxylic acid (55a, 18 mg, 0.05 mmol) in DMF (0.5 mL) was added DIEA (6 mg, 0.05 mmol) and HATU (19 mg, 0.05 mmol). After stirring for 60 min, 4-chlorobenzylamine (7 mg, 0.05 mmol) was added and the mixture was stirred at rt overnight, then purified by reversed-phase HPLC to provide the product (10 mg) as a TFA salt. ¹H NMR (400 MHz, DMSO-d₆) δ ppm: 3.90 (s, 3H), 3.95 (s, 3H), 3.97 (s, 3H), 4.49 (d, J=6.06 Hz, 2H), 7.36-7.45 (m, 4H), 7.52 (s, 1H), 7.71 (s, 1H), 8.39 (s, 1H), 9.06 (t, J=6.19 Hz, 1H), 13.28 (s, 1H); MS m/z (MH⁺) 514.3

### Example 57

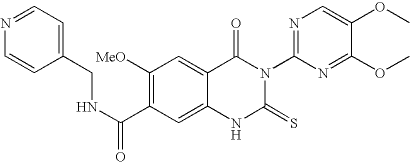

### 3-[4,5-Dimethoxy-2-pyrimidinyl]-6-methoxy-4-oxo-N-(4-pyridinylmethyl)-2-thioxo-1,2,3,4-tetrahydro-7-quinazolinecarboxamide

To a stirred solution of 3-[4,5-dimethoxy-2-pyrimidinyl]-6-methoxy-4-oxo-2-thioxo-1,2,3,4-tetrahydro-7-quinazolinecarboxylic (55a, 27 mg, 0.07 mmol, 1 eq.) in dry DMF (1 mL) were added (4-pyridinylmethyl)amine (8 mg, 0.08 mmol, 1.1 eq.), DIEA (27 mL, 0.15 mmol, 2.2 eq.), and HATU (29 mg, 0.08 mmol, 1.1 eq.) in order. The mixture was stirred at rt overnight then diluted with ethyl acetate, washed with water, brine, dried (MgSO4) and evaporated. The residue in DMSO was purified on HPLC under acidic condition (5-20% gradient). Yield: 19 mg, 58%; LC/MS: M+1=481; ¹H-NMR (400 MHz, CDCl₃) δ ppm: 9.31 (s, 1H), 8.72 (m, 2H), 8.21 (s, 1H), 7.79 (m, 2H), 7.58 (s, 1H), 5.52 (s, 1H), 5.33 (s, 1H), 4.08 (s, 3H), 4.01 (s, 6H),

### Example 58

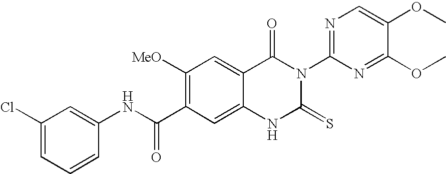

### N-(3-Chlorophenyl)-3-[4,5-dimethoxy-2-pyrimidinyl]-6-methoxy-4-oxo-2-thioxo-1,2,3,4-tetrahydro-7-quinazolinecarboxamide

To a stirred solution of 3-[4,5-dimethoxy-2-pyrimidinyl]-6-methoxy-4-oxo-2-thioxo-1,2,3,4-tetrahydro-7-quinazolinecarboxylic acid (55a, 23 mg, 0.06 mmol, 1 eq.) in dry DMF (1 mL) were added 3-chloroaniline (8 mg, 0.7 mmol, 1.1 eq.), DIEA (23 μL, 0.13 mmol, 2.2 eq.), and HATU (25 mg, 0.07 mmol, 1.1 eq.) in order. The mixture was stirred at rt overnight, diluted with ethyl acetate, washed with water, brine, dried (MgSO4) and evaporated. The residue in DMSO was purified on HPLC under acidic condition (30-60% gradient). Yield: 9 mg, 31%; LC/MS: M+1=500; ¹H-NMR (400 MHz, CDCl₃) δ ppm: 8.19 (s, 1H), 8.12 (s, 1H), 7.81 (s, 1H), 7.78 (s, 1H), 7.55 (d, 1H), 7.31 (t, 1H), 7.17 (d, 1H), 4.18 (s, 3H), 4.09 (s, 3H), 4.02 (s, 3H)

### Example 59

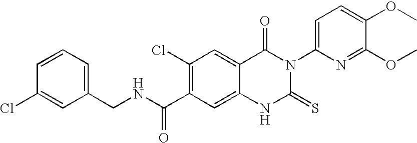

### 6-Chloro-N-(3-chlorobenzyl)-3-[5,6-dimethoxy-2-pyridinyl]-4-oxo-2-thioxo-1,2,3,4-tetrahydro-7-quinazolinecarboxamide

### a) dimethyl 2-amino-5-chloro-1,4-benzenedicarboxylate

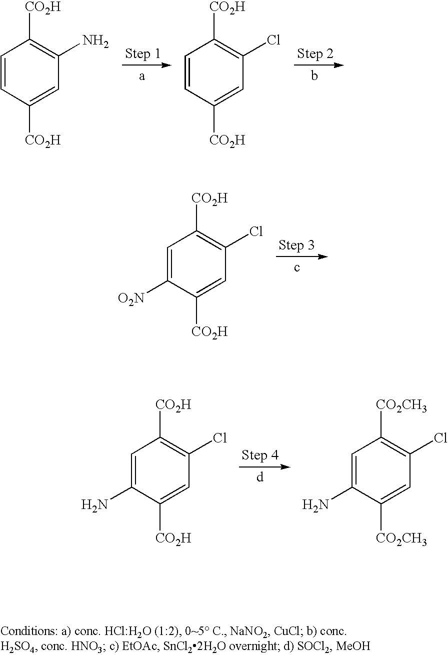

Step 1: To a suspension of 2-amino terephthalic acid (10 g, 55 mmole) in conc. hydrochloric acid (14.3 ml) and water (28.6 ml) a solution of sodium nitrite (3.8 g, 55 mmole) in water (18 ml) was added between 0-5° C. over a period of 15 to 20 minutes. After stirring at 0-5° C. for another 30 minutes, the reaction mixture was slowly added to cold cuprous chloride (12 g, 122 mmole) solution in conc. hydrochloric acid (60 ml) under stirring. Reaction mixture was then allowed to come to RT and stirred for 3 h. Mixture was filtered and the solid obtained was washed with ice-cold water (25 ml). The crude solid was dried under vacuum and then crystallized from water and ethanol (9:1) to get 8 g of 2-chloro terephthalic acid as an off-white solid (73%). ¹H NMR in CD₃OD-d₄δ ppm: 7.87 (1H, d, J=7.8 Hz, Ar—H), 7.98 (1H, d, J=7.8 Hz, Ar—H), 8.07 (1H, s, Ar—H).

Step 2: 2-Chloro terephthalic acid (8 g, 40 mmole) was dissolved in conc. sulfuric acid (33 ml) under ice-cooling. To the cold reaction mixture, 1:1 mixture of conc. nitric acid (3.6 ml) and conc. sulfuric acid (3.6 ml) was added slowly between 0-5° C. over a period of 15 minutes. After addition reaction mixture was heated at 100° C. for 2 h after which TLC revealed disappearance of starting material. Mixture was then cooled to rt and quenched with cold water (250 ml). Precipitated solid was filtered and washed with cold water (25 ml) and dried under vacuum to get 6 g of product (61%). ¹H NMR in CD₃OD-d₄δ ppm: 7.93 (1H, s Ar—H) 8.34 (1H, s, Ar—H).

Step 3: Mixture of 2-chloro-5-nitroterephthalic acid (10 g, 41 mmole) and stannous chloride dihydrate (27.5 g, 132 mmole) in ethyl acetate (375 ml) was stirred overnight at ambient temperature. TLC indicated disappearance of starting material. Mixture was diluted with ethyl acetate (200 ml) and washed with saturated aq. sodium chloride solution (75 ml). Ethyl acetate layer was evaporated and resulting residue was treated with 5% aq. sodium bicarbonate solution till the pH was ˜7. The semi-solid reaction mass was then stirred with ethyl acetate (500 ml) and filtered. The organic layer of the biphasic filtrate was separated, dried and evaporated to get 9 g of crude product. This was dissolved in methanol (200 ml), treated with charcoal, filtered and evaporated to yield 8.1 g of 2-amino-5-chloro-1,4-benzenedicarboxylic acid (92%) as yellow solid. ¹H NMR in CD₃OD-d₄δ ppm: 6.75 (1H, s ArH) 7.76 (1H, s, ArH).

Step 4: To a stirred solution of 2-amino-5-chloro-1,4-benzenedicarboxylic acid (2.16 g, 10 mmol, 1 eq.) in methanol (25 mL) cooled in an ice bath, thionyl chloride (3.57 g, 30 mmol, 3.0 eq.) was added dropwise. After stirring the mixture at rt for 3 hours, the reaction was checked with LCMS. Reaction mixture was concentrated, diluted with ethyl acetate (75 mL) and water (10 mL). The organic phase was dried over anhydrous sodium sulfate, filtered and concentrated to give the title product which was used without purification. Yield: 0.307 g, 12%; LC/MS: M+1=244

### 59b) Dimethyl 2-chloro-5-isothiocyanato-1,4-benzenedicarboxylate

To a stirred solution of dimethyl 2-amino-5-chloro-1,4-benzenedicarboxylate (59a, 307 mg, 1.26 mmol, 1 eq.) in sat sodium bicarbonate (25 mL) and chloroform (25 mL) was slowly added thiophosgene (174 mg, 1.51 mol, 1.2 eq.) and the mixture was stirred at rt for 2.5 hours. Phases were separated and the aqueous was extracted with DCM (3×25 mL). The combined organics were dried over anhydrous sodium sulfate, filtered and concentrated to give the title product which was used without purification. Yield: 0.43 g, 100%; LC/MS: M+1=286

### 59c) 6-Chloro-3-[5,6-dimethoxy-2-pyridinyl]-4-oxo-2-thioxo-1,2,3,4-tetrahydro-7-quinazolinecarboxylic acid

The mixture of dimethyl 2-chloro-5-isothiocyanato-1,4-benzenedicarboxylate (59b, 0.359 g, 1.26 mmol, 1 eq.) and 5,6-dimethoxy-2-pyridinamine (0.194 g, 1.26 mmol, 1.0 eq.) in THF (5 mL) was heated at 50° C. for 2 hours, then continued stirring overnight. After adding 10% sodium hydroxide solution, the mixture was monitored with LCMS, and stirred for two hours then concentrated to dryness and dissolved with DMSO. This was purified on HPLC (Gilson, 10-90% gradient system in 8 minutes) under acidic condition to give 223 mg of the title product with 100% purity. Yield: 223 mg, 45%; LC/MS: M+1=393.7

### 59d 6-Chloro-N-(3-chlorobenzyl)-)-3-[5,6-dimethoxy-2-pyridinyl]-4-oxo-2-thioxo-1,2,3,4-tetrahydro-7-quinazolinecarboxamide

3-Chlorobenzylamine (23.5 mg, 0.165 mmol, 1.1 eg.), HATU (62.7 mg, 0.165 mmol, 1.1 eq.) and DIEA (21.5 mg, 0.165 mmol, 1.1 eq.) were added to a stirred solution of crude 6-chloro-3-(5,6-dimethoxy-2-pyridinyl)-4-oxo-2-thioxo-1,2,3,4-tetrahydro-7-quinazolinecarboxylic acid (59c, 59 mg, 0.15 mmol, 1.0 eq.) in dry DMF (3 mL). The mixture was stirred at rt overnight. Reaction mixture was dropped into cold water (35 mL) and the solid collected, dissolved in DMSO, filtered and purified on HPLC (15-75% gradient in 8 minutes). Yield 12 mg.: 16%; LC/MS: M+1=517; 1H NMR (400 MHz, DMSO-d₆) δ ppm 3.78 (s, 3H) 3.86 (s, 3H) 4.51 (d, J=6.06 Hz, 2H) 7.02 (d, J=8.08 Hz, 1H) 7.33-7.38 (m, 2H) 7.40-7.48 (m, 4H) 7.97 (s, 1H) 9.34 (t, J=6.06 Hz, 1H), 13.26 (s, 1H)

### Example 60

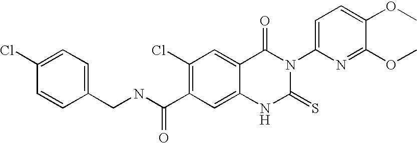

### 6-Chloro-N-(4-chlorobenzyl)-3-[5,6-dimethoxy-2-pyridinyl]-4-oxo-2-thioxo-1,2,3,4-tetrahydro-7-quinazolinecarboxamide

The 4-chlorobenzylamine (23.5 mg, 0.165 mmol, 1.1 eq.), HATU (62.7 mg, 0.165 mmol, 1.1 eq.) and DIEA (21.5, 0.165 mmol, 1.1 eq.) were added to a stirred solution of the crude 6-chloro-3-(5,6-dimethoxy-2-pyridinyl)-4-oxo-2-thioxo-1,2,3,4-tetrahydro-7-quinazolinecarboxylic acid (59c, 59 mg, 0.15 mmol, 1.0 eq.) in dry DMF (3 mL). The mixture was stirred at rt overnight, added to clod water (35 mL) and the precipitated solid filtered off. This solid in DMSO was filtered and purified on HPLC under neutral condition (15-75% gradient in 8 minutes). Yield: 20 mg., 23%; LC/MS: M+1=517; 1H NMR (400 MHz, DMSO-d₆) δ ppm 3.78 (s, 3H) 3.86 (s, 3H) 4.48 (d, J=6.06 Hz, 2H) 7.02 (d, J=8.08 Hz, 1H) 7.39-7.48 (m, 6H) 7.96 (s, 1H) 9.34 (m, J=6.06 Hz, 1H), 13.24 (s, 1H)

### Example 61

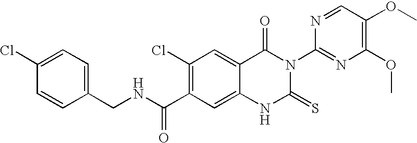

### 6-Chloro-N-(4-chlorobenzyl)-3-[4,5-dimethoxy-2-pyrimidinyl]-4-oxo-2-thioxo-1,2,3,4-tetrahydro-7-quinazolinecarboxamide

### 61a) 6-Chloro-3-[4,5-dimethoxy-2-pyrimidinyl]-4-oxo-2-thioxo-1,2,3,4-tetrahydro-7-quinazolinecarboxylic acid

To a solution of dimethyl 2-chloro-5-isothiocyanato-1,4-benzenedicarboxylate (59b, 100 mg, 0.35 mmol) in DMF (0.7 mL) was added 4,5-dimethoxy-2-pyrimidinamine (54 mg, 0.35 mmol). The mixture was stirred at 60° C. overnight, then 10% sodium hydroxide (0.5 mL) was added and the mixture was stirred at rt for 2 hours, acidified with 6N hydrochloric acid to pH ˜4 and concentrated to a residue. The residue was washed with water, centrifuged, and the supernatent was discarded. The residue was dissolved in DMSO, filtered, and purified by reversed-phase HPLC to furnish the title compound (25 mg). MS m/z (MH⁺) 394.7

### 61b) 6-Chloro-N-(4-chlorobenzyl)-3-[4,5-dimethoxy-2-pyrimidinyl]-4-oxo-2-thioxo-1,2,3,4-tetrahydro-7-quinazolinecarboxamide

To a solution of 6-chloro-43-[4,5-dimethoxy-2-pyrimidinyl]-oxo-2-thioxo-1,2,3,4-tetrahydro-7-quinazolinecarboxylic acid (61a, 20 mg, 0.05 mmol) in DMF (0.5 mL) was added DIEA (8 mg, 0.06 mmol) and HATU (21 mg, 0.06 mmol). After stirring for 15 min, 4-chlorobenzylamine (7 mg, 0.05 mmol) was added and the mixture was stirred at rt overnight, then purified by reversed-phase HPLC to provide the product (16.5 mg) as a TFA salt. ¹H NMR (400 MHz, DMSO-d₆) δ ppm: 3.91 (s, 3H), 3.97 (s, 3H), 4.49 (d, J=6.06 Hz, 2H), 7.39-7.47 (m, 5H), 8.00 (s, 1H), 8.39 (s, 1H), 9.33 (t, J=6.06 Hz, 1H), 13.40 (br. s., 1H); MS m/z (MH⁺) 518.1

### Example 62

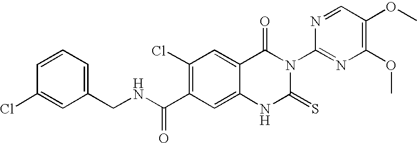

### 6-Chloro-N-(3-chlorobenzyl)-3-[4,5-dimethoxy-2-pyrimidinyl]-4-oxo-2-thioxo-1,2,3,4-tetrahydro-7-quinazolinecarboxamide

To a solution of 6-chloro-3-[4,5-dimethoxy-2-pyrimidinyl]-4-oxo-2-thioxo-1,2,3,4-tetrahydro-7-quinazolinecarboxylic acid (61a, 21 mg, 0.05 mmol) in DMF (0.5 mL) was added DIEA (8 mg, 0.06 mmol) and HATU (22 mg, 0.06 mmol). After stirring for 15 min, 3-chlorobenzylamine (8 mg, 0.05 mmol) was added and the mixture was stirred at rt overnight, then purified by reversed-phase HPLC to provide the product (13.8 mg) as a TFA salt. ¹H NMR (400 MHz, DMSO-d₆) δ ppm: 3.91 (s, 3H), 3.97 (s, 3H), 4.51 (d, J=5.81 Hz, 2H), 7.33-7.48 (m, 5H), 8.01 (s, 1H), 8.39 (s, 1H), 9.36 (t, J=6.06 Hz, 1H), 13.42 (br. s., 1H); MS m/z (MH⁺) 518.1

### Example 63

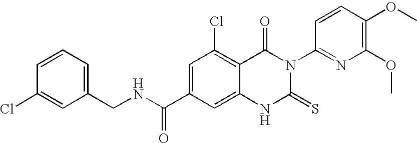

### 5-Chloro-N-(3-chlorobenzyl)-3-[5,6-dimethoxy-2-pyridinyl]-4-oxo-2-thioxo-1,2,3,4-tetrahydro-7-quinazolinecarboxamide

### 63a) dimethyl 2-amino-6-chloro-1,4-benzenedicarboxylate

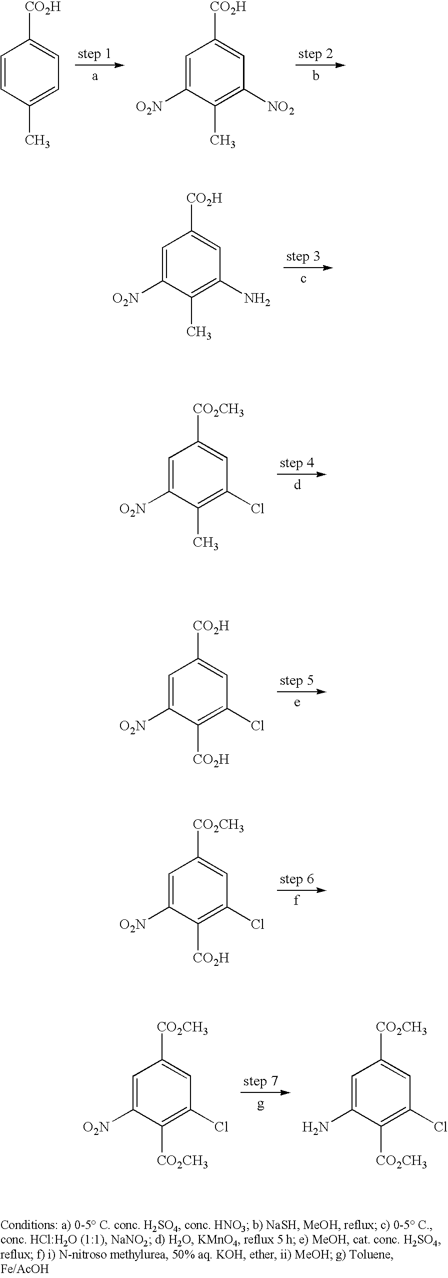

Step 1: 4-Methyl benzoic acid (50 g, 368 mmole) was dissolved in conc. sulfuric acid (500 ml) and conc. nitric acid (200 ml) was added slowly 18-20° C. over a period of 3 h. After addition, reaction mixture was then allowed to come to room temperature and stirred for 2 h. During this period, the dark viscous reaction mixture turned yellow in color with some amount of yellow solid precipitation. TLC revealed disappearance of starting material. Reaction mixture was poured over crushed ice water (1 kg) and stirred for 30 minutes. Pale yellow solid obtained was filtered, washed with water (100 ml) and dried under vacuum to yield 70 g of 4-methyl-3,5-dinitrobenzoic acid (83%). ¹H-NMR in DMSO-d₆δ ppm: 2.52 (3H, s, CH₃), 8.58 (2H, s, Ar—H).

Step 2: A solution of 4-methyl-3,5-dinitrobenzoic acid (70 g, 307 mmole) in methanol (750 ml) was warmed to 60° C. and a 30% solution of sodium hydrogen sulfide (133 ml, 539 mmole) was added slowly over a period of 30 minutes. The reaction mixture was refluxed for 3 h. Reaction mixture was evaporated to dryness and resulting residue was treated with water (500 ml). The mixture was acidified to pH 3 using dil. HCl. The precipitated product was extracted with ethyl acetate (3×250 ml) and the ethyl acetate layer was dried and evaporated to yield 50 g of 3-amino-4-methyl-5-nitrobenzoic acid as yellow solid (83%). ¹H-NMR in DMSO-d₆δ ppm: 2.14 (3H, s, CH₃), 5.85 (2H, bs, NH₂), 7.41 (1H, s, Ar—H), 7.56 (1H, s, Ar—H), 13.31 (1H, bs, COON).

Step 3: To a suspension of 3-amino-4-methyl-5-nitrobenzoic acid (25 g, 127 mmole) in a mixture of conc. hydrochloric acid (100 ml) and water (100 ml), a solution of sodium nitrite (9.3 g, 135 mmole) in water (25 ml) was added between 0-5° C. over a period of 15 to 20 minutes. After stirring at 0-5° C. for another 30 minutes, reaction mixture was slowly added to cold cuprous chloride (27.8 g, 280 mmole) solution in conc. hydrochloric acid (60 ml) under stirring. Reaction mixture was allowed to come to room temperature and stirred for 1 h. Precipitated solid was filtered, washed with ice-cold water (25 ml) and dried under vacuum to yield 21.5 g of 3-chloro-4-methyl-5-nitrobenzoic acid as off-white solid (78%). ¹H-NMR in CD₃OD-d₄δ ppm: 2.58 (3H, s, CH₃), 8.22 (1H, d, J=1.4 Hz, Ar—H), 8.23 (1H, d, J=1.4 Hz, Ar—H).

Step 4: A mixture of 3-chloro-4-methyl-5-nitrobenzoic acid (21.5 g, 100 mmole) and KMnO₄(31.6 g; 200 mmole) in water (475 ml) was refluxed for 4 h under stirring. After the disappearance of KMnO₄ color, TLC showed the presence of starting material. Additional KMnO₄ (15.8 g; 100 mmole) was added and refluxing continued for 4 h. When TLC revealed the disappearance of starting material, the reaction mixture was cooled to room temperature and filtered through celite. The filtrate was concentrated to ca. 150 ml on a rotavap. The concentrated aqueous mixture was cooled to 0-5° C. and acidified to pH 2 with conc. HCl. The precipitated solid was filtered and washed with water to yield 12 g of 2-chloro-6-nitroterephthalic acid as a colorless solid (49%). ¹H-NMR in DMSO-d₆δ ppm: 8.35 (1H, s, ArH) 8.49 (1H, s, ArH).

Step 5: To a suspension of 2-chloro-6-nitroterephthalic acid (12 g, 48 mmole) in methanol (300 ml) was added conc. sulfuric acid (7.5 ml) at ambient temperature and the mixture was refluxed for 18 h. The reaction mixture was concentrated on a rotavap and the resulting residue was dissolved in ethyl acetate (25 ml) and washed with water (2×20 ml). The organic phase was dried over anhydrous Na₂SO₄and evaporated under vacuum to yield 10 g of 2-chloro-6-nitro-terephthalic acid 4-methyl ester as colorless solid (80%). ¹H-NMR in CD₃OD-d₄δ ppm: 4.0 (3H, s, COOCH₃), 8.44 (1H, d, J=1.6 Hz, Ar—H), 8.67 (1H, d, J=1.4 Hz, Ar—H).

Step 6: To a solution of 2-chloro-6-nitro-terephthalic acid 4-methyl ester (10 g, 38 mmole) in methanol (200 ml), an ethereal solution of diazomethane (100 ml) [prepared from 50% aq. KOH (56 ml) and N-nitroso methylurea (14 g, 135 mmole)] was added between 0-5° C. over a period of 30 minutes. The reaction mixture was stirred between 0-5° C. for 1 h and then allowed to come to ambient temperature. Excess of diazomethane was quenched by adding acetic acid. The reaction mixture was dried and concentrated to yield 10 g of dimethyl 2-chloro-6-nitroterephthalate (96%) as a colorless solid. ¹H NMR in CDCl₃δ ppm: 4.0 (3H, s, COOCH₃), 4.03 (3H, s, COOCH₃), 8.36 (1H, d, J=1.4 Hz, Ar—H), 8.71 (1H, d, J=1.4 Hz, Ar—H).

Step 7: To a solution of dimethyl 2-chloro-6-nitroterephthalate (9.5 g, 35 mmole) in toluene (400 ml), iron (20 g, 357 mmole) was added at ambient temperature and mixture was heated to reflux. Under reflux condition, acetic acid (33 ml) was added slowly over a period of 1 h and refluxing continued for 2 h. Another lot of acetic acid (33 ml) was added over a period of 1 h and refluxing continued for 2 h. As TLC revealed presence of the starting material, another lot of acetic acid (33 ml) was added over a period of 1 h and refluxing continued for 2 h. Upon complete disappearance of starting material, mixture was cooled to room temperature and filtered through celite. Filtrate was concentrated on a rotavap and resulting residue was subjected to column chromatography purification over silica gel to yield 5 g of dimethyl 2-amino-6-chloroterephthalate as yellow solid (59%). ¹H NMR in CDCl₃δ ppm: 3.89 (3H, s, COOCH₃), 3.94 (3H, s, COOCH₃), 6.03 (2H, s, NH₂), 7.18 (1H, s, Ar—H), 7.3 (1H, s, Ar—H).

### 63b) Dimethyl 2-chloro-6-isothiocyanato-1,4-benzenedicarboxylate

To a biphasic mixture of dimethyl 2-amino-6-chloro-1,4-benzenedicarboxylate (63a, 1.00 g, 4.10 mmol) in chloroform (12 mL) and sat aqueous sodium bicarbonate (12 mL), thiophosgene (566 mg, 4.92 mmol) was added dropwise. The mixture was stirred vigorously at rt overnight. The phases were separated and the aqueous layer was extracted with DCM. The combined organic layer was dried (MgSO₄), filtered, and concentrated to furnish the title compound (1.17 g, 4.10 mmol) which was used without purification.

### 63c) 5-Chloro-3-[5,6-dimethoxy-2-pyridinyl]-4-oxo-2-thioxo-1,2,3,4-tetrahydro-7-quinazolinecarboxylic acid

To a solution of dimethyl 2-chloro-6-isothiocyanato-1,4-benzenedicarboxylate (63b, 250 mg, 0.88 mmol) in DMF (2 mL) was added 4,5-bis(methyloxy)-2-pyridinamine (136 mg, 0.88 mmol). The mixture was stirred at 60° C. overnight, then 10% sodium hydroxide (1 mL) was added and the mixture was stirred at rt for 2 hours. The mixture was acidified with 6N hydrochloric acid to pH ˜4 and the resulting precipitate was filtered and washed with water, then dried in vacuo to furnish the title compound (269 mg, 0.68 mmol) which was used without purification. MS m/z (MH⁺) 393.7

### 63d) 5-Chloro-N-(3-chlorobenzyl)-3-[5,6-dimethoxy-2-pyridinyl]-4-oxo-2-thioxo-1,2,3,4-tetrahydro-7-quinazolinecarboxamide

To a solution of 5-chloro-3-[5,6-dimethoxy-2-pyridinyl]-4-oxo-2-thioxo-1,2,3,4-tetrahydro-7-quinazolinecarboxylic acid (63c, 100 mg, 0.25 mmol) in DMF (1.3 mL) was added DIEA (36 mg, 0.28 mmol) and HATU (106 mg, 0.28 mmol). After stirring for 30 min, 3-chlorobenzylamine (35 mg, 0.25 mmol) was added and the mixture was stirred at rt overnight, then purified by reversed-phase HPLC to provide the product (27.8 mg) as a TFA salt. ¹H NMR (400 MHz, DMSO-d₆) δ ppm: 3.79 (s, 3H), 3.86 (s, 3H), 4.49 (d, J=5.81 Hz, 2H), 7.01 (d, J=8.08 Hz, 1H), 7.30-7.42 (m, 4H), 7.46 (d, J=8.08 Hz, 1H), 7.85 (dd, J=11.24, 1.39 Hz, 2H), 9.45 (t, J=5.94 Hz, 1H), 13.26 (br. s., 1H); MS m/z (MH⁺) 517.1

### Example 64

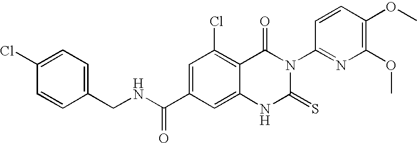

### 5-Chloro-N-(4-chlorobenzyl)-3-[5,6-dimethoxy-2-pyridinyl]-4-oxo-2-thioxo-1,2,3,4-tetrahydro-7-quinazolinecarboxamide

To a solution of 5-chloro-3-[5,6-dimethoxy-2-pyridinyl]-4-oxo-2-thioxo-1,2,3,4-tetrahydro-7-quinazolinecarboxylic acid (63c, 100 mg, 0.25 mmol) in DMF (1.3 mL) was added DIEA (36 mg, 0.28 mmol) and HATU (106 mg, 0.28 mmol). After stirring for 30 min, 4-chlorobenzylamine (35 mg, 0.25 mmol) was added and the mixture was stirred at rt overnight, then purified by reversed-phase HPLC to provide the product (5.1 mg) as a TFA salt. ¹H NMR (400 MHz, DMSO-d₆) δ ppm: 3.79 (s, 3H), 3.85 (s, 3H), 4.48 (d, J=5.81 Hz, 2H), 7.01 (d, J=8.08 Hz, 1H), 7.35-7.44 (m, 4H), 7.46 (d, J=8.34 Hz, 1H), 7.85 (dd, J=10.61, 1.52 Hz, 2H), 9.45 (t, J=5.94 Hz, 1H), 13.26 (s, 1H); MS m/z (MH⁺) 517.1

### Example 65

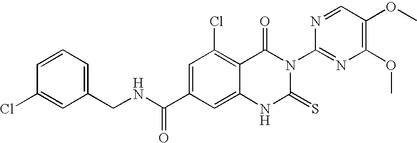

### 5-Chloro-N-(3-chlorobenzyl)-3-[4,5-dimethoxy-2-pyrimidinyl]-4-oxo-2-thioxo-1,2,3,4-tetrahydro-7-quinazolinecarboxamide

### 65a) 5-Chloro-3-[4,5-dimethoxy-2-pyrimidinyl]-4-oxo-2-thioxo-1,2,3,4-tetrahydro-7-quinazolinecarboxylic acid

To a solution of dimethyl 2-chloro-6-isothiocyanato-1,4-benzenedicarboxylate (63b, 250 mg, 0.88 mmol) in DMF (1 mL) was added 4,5-dimethoxy-2-pyrimidinamine (136 mg, 0.88 mmol). The mixture was stirred at 60° C. overnight, then 10% sodium hydroxide (1 mL) was added and the mixture was stirred at rt for 2 hours. The mixture was acidified with 6N hydrochloric acid to pH ˜4 and the resulting precipitate was filtered and washed with water, then dried in vacuo to furnish the title compound (305 mg, 0.77 mmol) which was used without purification. MS m/z (MH⁺) 394.8

### 65b) 5-Chloro-N-(3-chlorobenzyl)-3-[4,5-dimethoxy-2-pyrimidinyl]-4-oxo-2-thioxo-1,2,3,4-tetrahydro-7-quinazolinecarboxamide

To a solution of 5-chloro-3-[4,5-dimethoxy-2-pyrimidinyl]-4-oxo-2-thioxo-1,2,3,4-tetrahydro-7-quinazolinecarboxylic acid (65a, 100 mg, 0.25 mmol) in DMF (1.2 mL) was added DIEA (36 mg, 0.28 mmol) and HATU (106 mg, 0.28 mmol). After stirring for 30 min, 3-chlorobenzylamine (35 mg, 0.25 mmol) was added and the mixture was stirred at rt overnight, then purified by reversed-phase HPLC to provide the product (38.7 mg) as a TFA salt. ¹H NMR (400 MHz, DMSO-d₆) δ ppm: 3.92 (s, 3H), 3.96 (s, 3H), 4.50 (d, J=5.81 Hz, 2H), 7.30-7.42 (m, 4H), 7.87 (d, J=2.27 Hz, 2H), 8.39 (s, 1H), 9.48 (s, 1H), 13.42 (br. s., 1H); MS m/z (MH⁺) 518.0

### Example 66

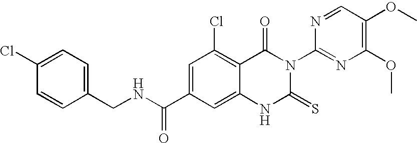

### 5-Chloro-N-(4-chlorobenzyl)-3-[4,5-dimethoxy-2-pyrimidinyl]-4-oxo-2-thioxo-1,2,3,4-tetrahydro-7-quinazolinecarboxamide

To a solution of 5-chloro-3-[4,5-dimethoxy-2-pyrimidinyl]-4-oxo-2-thioxo-1,2,3,4-tetrahydro-7-quinazolinecarboxylic acid (65a, 100 mg, 0.25 mmol) in DMF (1.2 mL) was added DIEA (36 mg, 0.28 mmol) and HATU (106 mg, 0.28 mmol). After stirring for 30 min, 4-chlorobenzylamine (35 mg, 0.25 mmol) was added and the mixture was stirred at rt overnight, then purified by reversed-phase HPLC to provide the product (40.5 mg) as a TFA salt. ¹H NMR (400 MHz, DMSO-d₆) δ ppm: 3.91 (s, 3H), 3.96 (s, 3H), 4.48 (d, J=5.81 Hz, 2H), 7.35-7.44 (m, 4H), 7.86 (d, J=3.03 Hz, 2H), 8.38 (s, 1H), 9.46 (s, 1H), 13.42 (br. s., 1H); MS m/z (MH⁺) 518.1

### Example 67

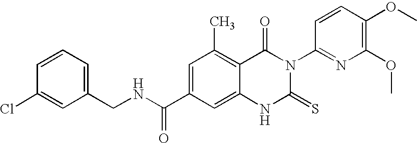

### N-(3-Chlorobenzyl)-3-[5,6-dimethoxy-2-pyridinyl]-5-methyl-4-oxo-2-thioxo-1,2,3,4-tetrahydro-7-quinazolinecarboxamide

### 67a) dimethyl 2-amino-6-methyl-1,4-benzenedicarboxylate

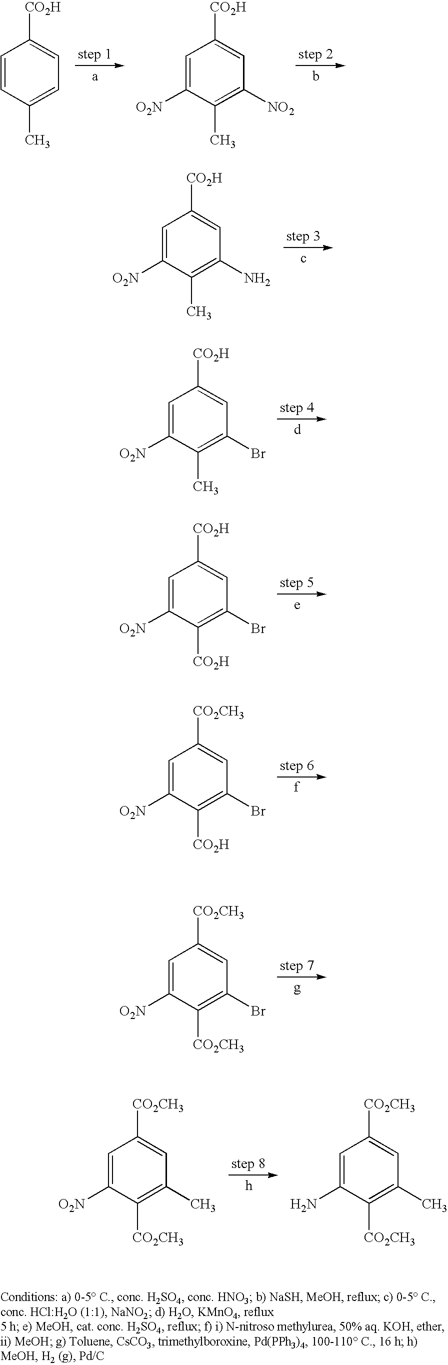

Step 1: 4-Methyl benzoic acid (50 g, 368 mmole) was dissolved in conc. sulfuric acid (500 ml) and conc. nitric acid (200 ml) was added slowly 18-20° C. over a period of 3 h. After addition, reaction mixture was then allowed to come to room temperature and stirred for 2 h. During this period, the dark viscous reaction mixture turned yellow in color with some amount of yellow solid precipitation. TLC revealed the disappearance of starting material. The reaction mixture was poured over crushed ice water (1 kg) and stirred for 30 minutes. The pale yellow solid obtained was filtered, washed with water (100 ml) and dried under vacuum to yield 70 g of 4-methyl-3,5-dinitrobenzoic acid (83%). ¹H-NMR in DMSO-d₆δ ppm: 2.52 (3H, s, CH₃), 8.58 (2H, s, Ar—H).

Step 2: A solution of 4-methyl-3,5-dinitrobenzoic acid (70 g, 307 mmole) in methanol (750 ml) was warmed to 60° C. and a 30% solution of sodium hydrogen sulfide (133 ml, 539 mmole) was added slowly over a period of 30 minutes. The reaction mixture was refluxed for 3 h. Reaction mixture was evaporated to dryness and resulting residue was treated with water (500 ml). The mixture was acidified to pH 3 using dil. HCl. The precipitated product was extracted with ethyl acetate (3×250 ml) and the ethyl acetate layer was dried and evaporated to yield 50 g of 3-amino-4-methyl-5-nitrobenzoic acid as yellow solid (83%). ¹H-NMR in DMSO-d₆δ ppm: 2.14 (3H, s, CH₃), 5.85 (2H, bs, NH₂), 7.41 (1H, s, Ar—H), 7.56 (1H, s, Ar—H), 13.31 (1H, bs, COON).

Step 3: To a suspension of 3-amino-4-methyl-5-nitrobenzoic acid (50 g, 254 mmole) in a mixture of conc. hydrochloric acid (350 ml) and water (350 ml) was added between 0-5° C. a solution of sodium nitrite (18.6 g, 270 mmole) in water (30 ml) over a period of 15 to 20 minutes. After stirring at 0-5° C. for another 30 minutes, the reaction mixture was then slowly added to a cold cuprous bromide (73.21 g, 516 mmole) solution in conc. hydrochloric acid (220 ml) with stirring. The reaction mixture was stirred at 0-5° C. for 30 minutes and then at room temperature for another 30 minutes. Finally, after stirring at 30-35° C. for 30 minutes, precipitated solid was filtered, washed with water (50 ml) and dried under vacuum to yield 58 g of 3-bromo-4-methyl-5-nitrobenzoic acid as pink solid (88%). ¹H-NMR in CD₃OD-d₄δ ppm: 2.6 (3H, s, CH₃), 8.21 (1H, d, J=1.4 Hz, Ar—H), 8.27 (1H, d, J=1.2 Hz, Ar—H).

Step 4: To a solution of KMnO₄(176.85 g, 1.12 mole) in water (2650 ml) was added at ambient temperature 3-bromo-4-methyl-5-nitrobenzoic acid (97 g, 373 mmole) and the mixture was then heated to reflux. After every 3 h, additional lots of KMnO4 (58.95 g, 373 mmole; 117.9 g, 746 mmole; 58.95 g, 373 mmole) were added and thereaftere refluxing continued for a further 5 h. The reaction mixture was cooled to room temperature and filtered through celite. The filtrate was concentrated to approximately 400 ml on a rotavap. The concentrated aqueous mixture was cooled to 0-5° C. and acidified to pH 2 with conc. HCl. The precipitated solid was filtered and washed with water (50 ml) to yield 85 g of 2-bromo-6-nitroterephthalic acid as an off-white solid after drying (79%). ¹H-NMR in DMSO-d₆δ ppm: 8.12 (1H, s, Ar—H) 8.31 (1H, s, Ar—H).

Step 5: To a suspension of 2-bromo-6-nitroterephthalic acid (80 g, 276 mmole) in methanol (2000 ml) was added at ambient temperature conc. sulfuric acid (25 ml) and the mixture refluxed for 10 h. The reaction mixture was filtered and concentrated on a rota-yap. The resulting residue was dissolved in ethyl acetate (800 ml) and washed with water (2×50 ml). The organic layer was dried over Na₂SO₄and evaporated under vacuum to yield 70 g of 2-bromo-6-nitro-terephthalic acid 4-methyl ester as colorless solid (83%). ¹H-NMR in CD₃OD-d₄δ ppm: 3.97 (3H, s, OCH₃), 8.35 (1H, d, J=1.2 Hz, Ar—H), 8.59 (1H, d, J=1.2 Hz, Ar—H).

Step 6: To a solution of 2-bromo-6-nitro-terephthalic acid 4-methyl ester (25 g, 82 mmole) in methanol (50 ml) maintained between 0-5° C. was added an ethereal solution of diazomethane (340 ml) [prepared from 50% aq. KOH (156 ml) and N-nitroso methylurea (34 g, 330 mmole)] over a period of 30 minutes. The reaction mixture was stirred between 0-5° C. for 1 h and then allowed to come to room temperature. Excess diazomethane was quenched by adding acetic acid. The reaction mixture was dried over Na₂SO₄and concentrated under vacuum to yield 19 g of dimethyl 2-bromo-6-nitroterephthalate (73%) as a colorless solid. ¹H-NMR in CDCl₃δ ppm: 4.01 (3H, s, OCH₃), 4.03 (3H, s, OCH₃), 8.36 (1H, s, Ar—H), 8.7 (1H, s, Ar—H).

Step 7: To a solution of dimethyl 2-bromo-5-nitroterephthalate (12 g; 38 mmole) in toluene (50 ml) maintained at ambient temperature were added cesium carbonate (36 g; 110 mmole), tetrakis(triphenylphosphine)palladium (4.32 g; 3.8 mmole) and trimethylboroxine (5.28 ml; 38 mmole) sequentially under an inert atmosphere (nitrogen). The mixture was heated to 100-110° C. for 8 h. Another lot of trimethylboroxine (5.28 ml; 38 mmole) was added and the mixture was heated at 100-110° C. for another 8 h. The reaction mass was cooled to ambient temperature and filtered. The filtrate was concentrated on a rotavap and the resulting brown solid (11 g) was purified by column chromatography over silica gel (2.5% ethyl acetate in hexane) to yield 5.8 g (60%) of dimethyl 2-methyl-6-nitroterephthalate as colorless solid. ¹H-NMR in CDCl₃δ ppm: 2.47 (3H, s, CH₃), 3.99 (6H, s, OCH₃), 8.19 (1H, s, Ar—H), 8.6 (1H, s, Ar—H).

Step 8: Mixture of dimethyl 2-methyl-6-nitroterephthalate (5.7 g; 22.4 mmoles), 5% Pd/C (0.7 g) in methanol (228 ml) was hydrogenated under a hydrogen pressure of 40 psi for 2 h in a Parr hydrogenator. The mixture was filtered under nitrogen and filtrate was concentrated to get 4.2 g (84%) of dimethyl 2-amino-6-methylterephthalate as yellow solid. ¹H NMR in DMSO-d₆δ ppm: 2.44 (3H, s, CH₃), 3.94 (3H, s, OCH₃), 3.96 (3H, s, OCH₃), 5.15 (2H, bs, NH₂), 8.45 (1H, d, J=1.0 Hz, Ar—H), 8.55 (1H, d, J=1.2 Hz, Ar—H).

### 67b) Dimethyl 2-isothiocyanato-6-methyl-1,4-benzenedicarboxylate

To a biphasic mixture of dimethyl 2-amino-6-methyl-1,4-benzenedicarboxylate (67a, 1.00 g, 4.48 mmol) in chloroform (12 mL) and sat aqueous sodium bicarbonate (12 mL), thiophosgene (618 mg, 5.38 mmol) was added dropwise. The mixture was stirred vigorously at rt overnight. The phases were separated and the aqueous layer was extracted with DCM. The combined organic layer was dried (MgSO₄), filtered, and concentrated to furnish the title compound (1.18 g, 4.45 mmol) which was used without purification.

### 67c) 3-[5,6-Dimethoxy-2-pyridinyl]-5-methyl-4-oxo-2-thioxo-1,2,3,4-tetrahydro-7-quinazolinecarboxylic acid

To a solution of dimethyl 2-isothiocyanato-6-methyl-1,4-benzenedicarboxylate (67b, 250 mg, 0.94 mmol) in DMF (2 mL) was added 4,5-dimethoxy-2-pyridinamine (145 mg, 0.94 mmol). The mixture was stirred at 60° C. overnight, then 10% sodium hydroxide (1 mL) was added and the mixture was stirred at rt for 2 hours. The mixture was acidified with 6N hydrochloric acid to pH ˜4 and the resulting precipitate was filtered and washed with water, then dried in vacuo to furnish the title compound (296 mg, 0.79 mmol) which was used without purification. MS m/z (MH⁺) 373.7

### 67d) N-(3-Chlorobenzyl)-3-[5,6-dimethoxy-2-pyridinyl]-5-methyl-4-oxo-2-thioxo-1,2,3,4-tetrahydro-7-quinazolinecarboxamide

To a solution of 3-[5,6-dimethoxy-2-pyridinyl]-5-methyl-4-oxo-2-thioxo-1,2,3,4-tetrahydro-7-quinazolinecarboxylic acid (67c, 100 mg, 0.27 mmol) in DMF (1.3 mL) was added DIEA (39 mg, 0.30 mmol) and HATU (113 mg, 0.30 mmol). After stirring for 30 min, 3-chlorobenzylamine (38 mg, 0.27 mmol) was added and the mixture was stirred at rt for 4 hours, then purified by reversed-phase HPLC to provide the product (79.2 mg) as a TFA salt. ¹H NMR (400 MHz, DMSO-d₆) δ ppm: 2.69 (s, 3H), 3.79 (s, 3H), 3.85 (s, 3H), 4.49 (d, J=5.81 Hz, 2H), 7.00 (d, J=8.08 Hz, 1H), 7.30-7.42 (m, 4H), 7.45 (d, J=8.34 Hz, 1H), 7.59 (s, 1H), 7.76 (s, 1H), 9.34 (t, J=6.06 Hz, 1H), 13.09 (s, 1H); MS m/z (MH⁺) 497.1

### Example 68

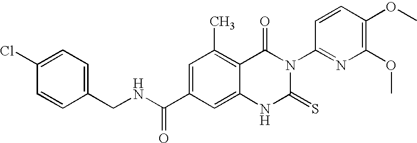

### N-(4-Chlorobenzyl)-3-[5,6-dimethoxy-2-pyridinyl]-5-methyl-4-oxo-2-thioxo-1,2,3,4-tetrahydro-7-quinazolinecarboxamide

To a solution of 3-[5,6-dimethoxy-2-pyridinyl]-5-methyl-4-oxo-2-thioxo-1,2,3,4-tetrahydro-7-quinazolinecarboxylic acid (67c, 100 mg, 0.27 mmol) in DMF (1.3 mL) was added DIEA (39 mg, 0.30 mmol) and HATU (113 mg, 0.30 mmol). After stirring for 30 min, 4-chlorobenzylamine (38 mg, 0.27 mmol) was added and the mixture was stirred at rt for 4 hours, then purified by reversed-phase HPLC to provide the product (42.1 mg) as a TFA salt. ¹H NMR (400 MHz, DMSO-d₆) δ ppm: 2.68 (s, 3H), 3.79 (s, 3H), 3.85 (s, 3H), 4.48 (d, J=5.81 Hz, 2H), 7.00 (d, J=8.08 Hz, 1H), 7.35-7.43 (m, 4H), 7.45 (d, J=8.34 Hz, 1H), 7.59 (s, 1H), 7.75 (s, 1H), 9.33 (s, 1H), 13.09 (s, 1H); MS m/z (MH⁺) 497.1

### Example 69

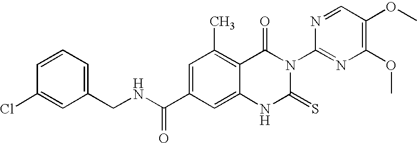

### N-(3-Chlorobenzyl)-3-[4,5-dimethyoxy-2-pyrimidinyl]-5-methyl-4-oxo-2-thioxo-1,2,3,4-tetrahydro-7-quinazolinecarboxamide

### 69a) 3-[4,5-Dimethoxy-2-pyrimidinyl]-5-methyl-4-oxo-2-thioxo-1,2,3,4-tetrahydro-7-quinazolinecarboxylic acid

To a solution of dimethyl 2-isothiocyanato-6-methyl-1,4-benzenedicarboxylate (67b, 200 mg, 0.75 mmol) in DMF (1 mL) was added 4,5-dimethoxy-2-pyrimidinamine (116 mg, 0.75 mmol). The mixture was stirred at 60° C. overnight, then 10% sodium hydroxide (1 mL) was added and the mixture was stirred at rt for 2 hours. The mixture was acidified with 6N hydrochloric acid to pH ˜4 and the resulting precipitate was filtered and washed with water, then dried in vacuo to furnish the title compound (237 mg, 0.63 mmol) which was used without purification. MS m/z (MH⁺) 374.8

### 69b N-(3-Chlorobenzyl)-) 3-[4,5-dimethoxy-2-pyrimidinyl]-5-methyl-4-oxo-2-thioxo-1,2,3,4-tetrahydro-7-quinazolinecarboxamide

To a solution of 3-[4,5-dimethoxy-2-pyrimidinyl]-5-methyl-4-oxo-2-thioxo-1,2,3,4-tetrahydro-7-quinazolinecarboxylic acid (69a, 100 mg, 0.27 mmol) in DMF (1.2 mL) was added DIEA (39 mg, 0.30 mmol) and HATU (113 mg, 0.30 mmol). After stirring for 30 min, 3-chlorobenzylamine (38 mg, 0.27 mmol) was added and the mixture was stirred at rt overnight, then purified by reversed-phase HPLC to provide the product (36.1 mg) as a TFA salt. ¹H NMR (400 MHz, DMSO-d₆) δ ppm: 2.70 (s, 3H), 3.91 (s, 3H), 3.96 (s, 3H), 4.50 (d, J=6.06 Hz, 2H), 7.30-7.42 (m, 4H), 7.62 (s, 1H), 7.77 (s, 1H), 8.37 (s, 1H), 9.36 (t, J=5.94 Hz, 1H), 13.24 (s, 1H); MS m/z (MH⁺) 498.1

### Example 70

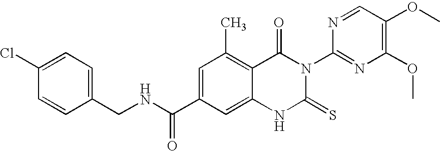

### N-(4-Chlorobenzyl)-3-[4,5-dimethoxy-2-pyrimidinyl]-5-methyl-4-oxo-2-thioxo-1,2,3,4-tetrahydro-7-quinazolinecarboxamide

To a solution of 3-[4,5-dimethoxy-2-pyrimidinyl]-5-methyl-4-oxo-2-thioxo-1,2,3,4-tetrahydro-7-quinazolinecarboxylic acid (69a, 100 mg, 0.27 mmol) in DMF (1.2 mL) was added DIEA (39 mg, 0.30 mmol) and HATU (113 mg, 0.30 mmol). After stirring for 30 min, 4-chlorobenzylamine (38 mg, 0.27 mmol) was added and the mixture was stirred at rt overnight, then purified by reversed-phase HPLC to provide the product (45.9 mg) as a TFA salt. ¹H NMR (400 MHz, DMSO-d₆) δ ppm: 2.69 (s, 3H), 3.91 (s, 3H), 3.96 (s, 3H), 4.48 (d, J=5.81 Hz, 2H), 7.35-7.44 (m, 4H), 7.61 (s, 1H), 7.76 (s, 1H), 8.37 (s, 1H), 9.35 (t, J=5.94 Hz, 1H), 13.24 (s, 1H); MS m/z (MH⁺) 498.1

### Example 71

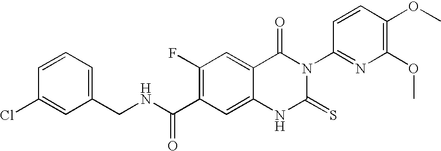

### N-(3-Chlorobenzyl)-3-[5,6-dimethoxy-2-pyridinyl]-6-fluoro-4-oxo-2-thioxo-1,2,3,4-tetrahydro-7-quinazolinecarboxamide

### 71a) dimethyl 2-amino-5-fluoro-1,4-benzenedicarboxylate

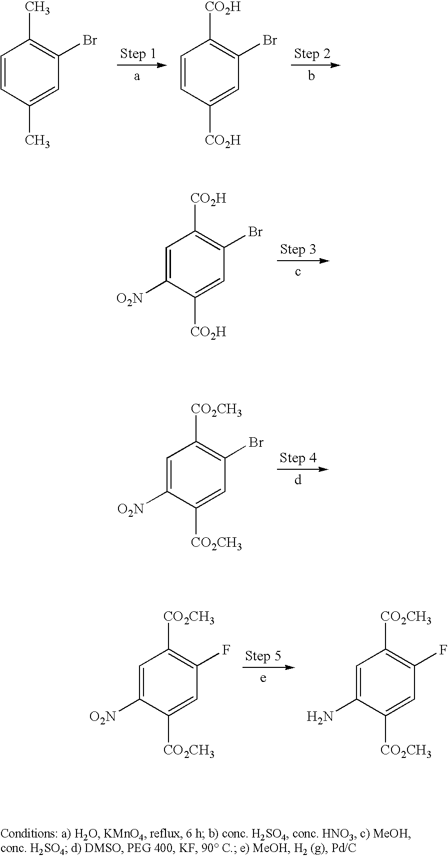

Step 1: A mixture of 2-bromo-p-xylene (18.5 g, 100 mmole) and KMnO₄(15.8 g; 100 mmole) in water (225 ml) was refluxed for 2 h under stirring. After the disappearance of KMnO₄-color, TLC showed the presence of starting material. Additional KMnO₄(15.8 g; 100 mmole) was added and refluxing continued for 2 h. TLC showed the presence of starting material, another lot of KMnO₄(15.8 g; 100 mmole) was added and refluxing continued for 2 h. TLC showed the presence of starting material, however, the reaction was worked up. Mixture was cooled to RT and filtered. Filtrate was extracted with ethyl acetate (2×25 ml). Ethyl acetate layer was dried and evaporated to recover 6.15 g (33%) of the starting material. The aqueous filtrate was concentrated to half volume on a rotavap. The concentrated aqueous mixture was cooled to 0-5° C. and acidified to pH 2 with conc. HCl. The precipitated solid was filtered and washed with water and dried to yield 11.39 g (47%) of 2-bromo terephthalic acid as a colorless solid. ¹H NMR in CD₃OD-d₄ δ ppm: 7.86 (1H, d, J=7.8 Hz, Ar—H), 8.05 (1H, dd, J=8.4 Hz & 1.6 Hz, Ar—H), 8.28 (1H, d, J=1.6 Hz, Ar—H).

Step 2: 2-Bromo terephthalic acid (13.8 g, 56.3 mmole) was slowly added under stirring to conc. H₂SO₄(78 ml) at 0-5° C. over 5 minutes. To the resulting mixture was added 1:1 mixture of conc. H₂SO₄and conc. HNO₃(15 ml) dropwise over 20 min. at 0-5° C. The mixture was then heated to 100° C. for 2 h. After cooling and stirring for 18 h at ambient temperature, mixture was poured into 100 g of ice-water. The resulting colorless solid was filtered and dried. The solid was recrystallized from ethanol to give 10.5 g (64%) of 2-bromo-5-nitroterephthalic acid. ¹H NMR in CD₃OD-d₄δ ppm: 3.96 (3H, s, OCH3) 4.01 (3H, s, OCH3) 8.16 (1H, s, ArH) 8.41 (1H, s, ArH).

Step 3: To a suspension of 2-bromo-5-nitroterephthalic acid (10.5 g; 36.2 mmole) in methanol (200 ml) was added dropwise conc H₂SO₄(5 ml) at rt. The mixture was refluxed for 18 h. TLC showed the disappearance of starting material and formation of non-polar product along with small amount of monoester. Methanol was distilled out on a rotavap and the resulting solid was stirred with water (25 ml), filtered and washed with water. The wet solid was dissolved in ethyl acetate (100 ml) and washed with saturated solution of NaHCO₃(25 ml). The organic layer was dried and evaporated to get 8 g (69%) of dimethyl 2-bromo-5-nitroterephthalate as a colorless solid. ¹H NMR in CD₃OD-d₄δ ppm: 3.96 (3H, s, OCH3) 4.01 (3H, s, OCH₃) 8.16 (1H, s, ArH) 8.41 (1H, s, ArH).

Step 4: A mixture of dimethyl 2-bromo-5-nitroterephthalate (8.5 g; 26.7 mmole), PEG 400 (1.92 g) and KF (5.35 g, 92.1 mmole) in DMSO (250 ml) was heated at 90° C. for 8 h. ¹H-NMR of the reaction mass showed disappearance of starting material along with the formation of a phenolic impurity. Reaction mixture was quenched with water (500 ml) and the mass was extracted with ethyl acetate (3×100 ml). Combined ethyl acetate extract was washed with water (25 ml), dried and evaporated to get an oily residue which after silica gel column chromatography purification gave 4.5 g of product contaminated with the phenolic impurity as revealed by ¹H-NMR. The column purified material was washed with 10% sodium hydroxide solution to yield 3.2 g of dimethyl 2-fluoro-5-nitroterephthalate as yellow solid (46%). ¹H-NMR, CDCl₃δ ppm: 3.96 (3H, s, COOCH₃), 3.99 (3H, s, COOCH₃), 7.45 (1H, d, J=9.2 Hz, Ar—H), 8.59 (1H, d, J=5.8 Hz, Ar—H).

Step 5: A mixture of dimethyl 2-fluoro-5-nitroterephthalate (0.5 g; 2 mmoles) in toluene (30 ml), containing iron (0.9 g, 16.2 mmole) was heated to reflux. Under reflux condition, acetic acid (0.3 ml) was added slowly over a period of 1 h and refluxing continued for 2 h. TLC revealed the presence of starting material, so another lot of acetic acid (0.3 ml) was added over a period of 1 h and refluxing continued for 2 h. Addition of acetic acid was repeated again, after which TLC revealed the disappearance of the starting material. The reaction mixture was cooled to rt and filtered through celite. The filtrate was concentrated on a rotavap and resulting residue was subjected to column chromatography purification over silica gel to yield 0.25 g of dimethyl 2-amino-5-fluoroterephthalate as a yellow solid (55%). ¹H-NMR, CDCl₃-d₄δ ppm: 3.88 (3H, s, COOCH₃), 3.91 (3H, s, COOCH₃), 5.6 (2H, br s, NH₂), 7.18 (1H, d, J=5.8 Hz, Ar—H), 7.36 (1H, d, J=11.8 Hz, Ar—H).

71b) Dimethyl 2-fluoro-5-isothiocyanato-1,4-benzenedicarboxylate

To a biphasic mixture of dimethyl 2-amino-5-fluoro-1,4-benzenedicarboxylate (71a, 400 mg, 1.76 mmol) in chloroform (8 mL) and sat aqueous sodium bicarbonate (5 mL), thiophosgene (263 mg, 2.29 mmol) was added dropwise. The mixture was stirred vigorously at rt overnight. The phases were separated and the aqueous layer was extracted with DCM. The combined organic layer was dried (MgSO₄), filtered, and concentrated to furnish the title compound (467 mg, 1.73 mmol) which was used without purification.

### 71c) 3-[5,6-Dimethoxy-2-pyridinyl]-6-fluoro-4-oxo-2-thioxo-1,2,3,4-tetrahydro-7-quinazolinecarboxylic acid

To a solution of dimethyl 2-fluoro-5-isothiocyanato-1,4-benzenedicarboxylate (71b, 200 mg, 0.74 mmol) in DMF (1.2 mL) was added 4,5-dimethoxy-2-pyridinamine (114 mg, 0.74 mmol). The mixture was stirred at rt for 4 hours, then 10% sodium hydroxide (0.6 mL) was added and the mixture was stirred at rt for 2 hours. The mixture was acidified with 6N hydrochloric acid to pH ˜4 and concentrated to a residue which was washed with water, centrifuged, and the supernatent discarded. The residue was dissolved in DMSO, filtered and purified by reversed-phase HPLC to furnish the title compound (190 mg). MS m/z (MH⁺) 377.9

### 71d N-(3-Chlorobenzyl)-)-3-[5,6-dimethoxy-2-pyridinyl]-6-fluoro-4-oxo-2-thioxo-1,2,3,4-tetrahydro-7-quinazolinecarboxamide

To a solution of 3-[5,6-dimethoxy-2-pyridinyl]-6-fluoro-4-oxo-2-thioxo-1,2,3,4-tetrahydro-7-quinazolinecarboxylic acid (71c, 60 mg, 0.16 mmol) in DMF (0.8 mL) was added DIEA (41 mg, 0.32 mmol) and HATU (122 mg, 0.32 mmol). After stirring for 60 min, 3-chlorobenzylamine (45 mg, 0.32 mmol) was added and the mixture was stirred at rt overnight, then purified by reversed-phase HPLC to provide the product (42.8 mg) as a TFA salt. ¹H NMR (400 MHz, DMSO-d₆) δ ppm: 3.78 (s, 3H), 3.86 (s, 3H), 4.51 (d, J=6.06 Hz, 2H), 7.02 (d, J=8.08 Hz, 1H), 7.31-7.44 (m, 4H), 7.46 (d, J=8.34 Hz, 1H), 7.65 (d, J=5.56 Hz, 1H), 7.80 (d, J=9.35 Hz, 1H), 9.29 (t, J=5.81 Hz, 1H), 13.24 (s, 1H); MS m/z (MH⁺) 501.0

### Example 72

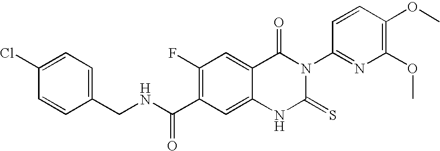

### N-(4-Chlorobenzyl)-3-[5,6-dimethoxy-2-pyridinyl]-6-fluoro-4-oxo-2-thioxo-1,2,3,4-tetrahydro-7-quinazolinecarboxamide

To a solution of 3-[5,6-dimethoxy-2-pyridinyl]-6-fluoro-4-oxo-2-thioxo-1,2,3,4-tetrahydro-7-quinazolinecarboxylic acid (71c, 60 mg, 0.16 mmol) in DMF (0.8 mL) was added DIEA (41 mg, 0.32 mmol) and HATU (122 mg, 0.32 mmol). After stirring for 60 minutes, 4-chlorobenzylamine (45 mg, 0.32 mmol) was added and the mixture was stirred at rt overnight, then purified by reversed-phase HPLC to provide the product (32.4 mg) as a TFA salt. ¹H NMR (400 MHz, DMSO-d₆) δ ppm: 3.78 (s, 3H), 3.86 (s, 3H), 4.49 (d, J=6.06 Hz, 2H), 7.02 (d, J=8.08 Hz, 1H), 7.35-7.48 (m, 5H), 7.64 (d, J=5.56 Hz, 1H), 7.79 (d, J=9.35 Hz, 1H), 9.27 (t, J=5.68 Hz, 1H), 13.24 (s, 1H); MS m/z (MH⁺) 501.2

### Example 73

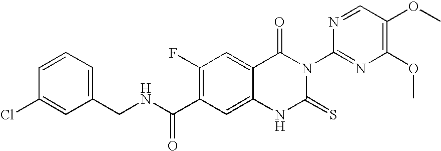

### N-(3-Chlorobenzyl)-3-[4,5-dimethoxy-2-pyrimidinyl]-6-fluoro-4-oxo-2-thioxo-1,2,3,4-tetrahydro-7-quinazolinecarboxamide

### 73a) 3-[4,5-Dimethoxy-2-pyrimidinyl]-6-fluoro-4-oxo-2-thioxo-1,2,3,4-tetrahydro-7-quinazolinecarboxylic acid

To a solution of dimethyl 2-fluoro-5-isothiocyanato-1,4-benzenedicarboxylate (71b, 200 mg, 0.74 mmol) in DMF (1.3 mL) was added 4,5-dimethoxy-2-pyrimidinamine (115 mg, 0.74 mmol). The mixture was stirred at 65° C. overnight, then 10% sodium hydroxide (0.6 mL) was added and the mixture was stirred at rt for 1 hr. The mixture was acidified with 6N hydrochloric acid to pH ˜4 and concentrated to a residue which was washed with water, centrifuged, and the supernatent was discarded. The residue was dissolved in DMSO, filtered, and purified by reversed-phase HPLC to furnish the title compound (130 mg). MS m/z (MH⁺) 378.9

### 73b) N-(3-Chlorobenzyl)-3-[4,5-dimethoxy-2-pyrimidinyl]-6-fluoro-4-oxo-2-thioxo-1,2,3,4-tetrahydro-7-quinazolinecarboxamide

To a solution of 3-[4,5-dimethoxy-2-pyrimidinyl]-6-fluoro-4-oxo-2-thioxo-1,2,3,4-tetrahydro-7-quinazolinecarboxylic acid (73a, 60 mg, 0.16 mmol) in DMF (0.8 mL) was added DIEA (41 mg, 0.32 mmol) and HATU (122 mg, 0.32 mmol). After stirring for 60 min, 3-chlorobenzylamine (45 mg, 0.32 mmol) was added and the mixture was stirred at rt overnight, then purified by reversed-phase HPLC to provide the product (53.5 mg) as a TFA salt. ¹H NMR (400 MHz, DMSO-d₆) δ ppm: 3.91 (s, 3H), 3.97 (s, 3H), 4.51 (d, J=6.06 Hz, 2H), 7.30-7.44 (m, 4H), 7.66 (d, J=5.56 Hz, 1H), 7.84 (d, J=9.35 Hz, 1H), 8.39 (s, 1H), 9.31 (t, J=5.94 Hz, 1H), 13.40 (s, 1H); MS m/z (MH⁺) 501.9

### Example 74

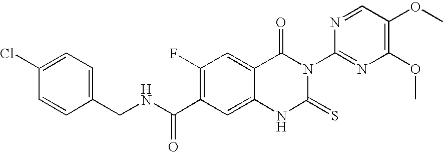

### N-(4-Chlorobenzyl)-3-[4,5-dimethoxy-2-pyrimidinyl]-6-fluoro-4-oxo-2-thioxo-1,2,3,4-tetrahydro-7-quinazolinecarboxamide

To a solution of 3-[4,5-dimethoxy-2-pyrimidinyl]-6-fluoro-4-oxo-2-thioxo-1,2,3,4-tetrahydro-7-quinazolinecarboxylic acid (73a, 60 mg, 0.16 mmol) in DMF (0.8 mL) was added DIEA (41 mg, 0.32 mmol) and HATU (122 mg, 0.32 mmol). After stirring for 60 min, 4-chlorobenzylamine (45 mg, 0.32 mmol) was added and the mixture was stirred at rt overnight, then purified by reversed-phase HPLC to provide the product (17.5 mg) as a TFA salt. ¹H NMR (400 MHz, DMSO-d₆) δ ppm: 3.91 (s, 3H), 3.97 (s, 3H), 4.49 (d, J=5.81 Hz, 2H), 7.36-7.46 (m, 4H), 7.65 (d, J=5.56 Hz, 1H), 7.83 (d, J=9.09 Hz, 1H), 8.39 (s, 1H), 9.28 (t, J=5.94 Hz, 1H), 13.39 (s, 1H); MS m/z (MH⁺) 501.9

### Example 75

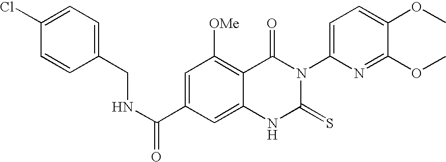

### N-(4-chlorobenzyl)-3-(5,6-dimethoxy-2-pyridinyl)-5-methoxy-4-oxo-2-thioxo-1,2,3,4-tetrahydro-7-quinazolinecarboxamide

### 75a) dimethyl 2-amino-6-(methyloxy)-1,4-benzenedicarboxylate

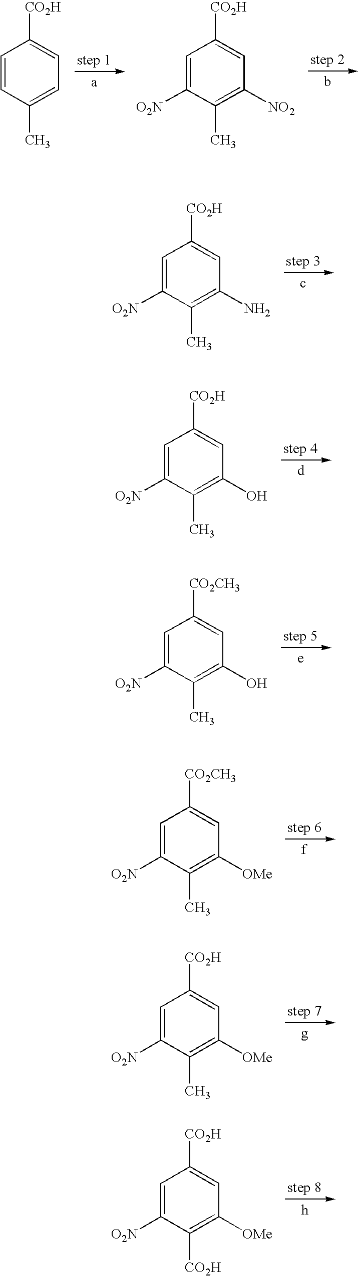

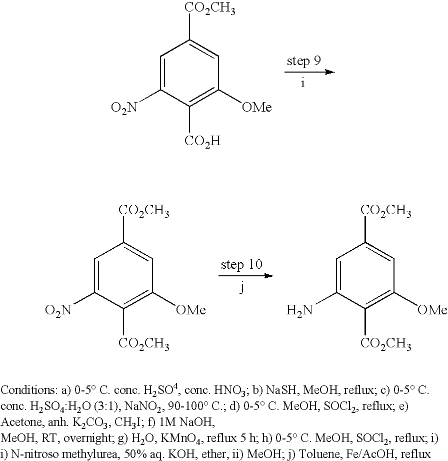

Step 1: 4-Methyl benzoic acid (50 g, 368 mmole) was dissolved in conc. sulfuric acid (500 ml) and conc. nitric acid (200 ml) was added slowly 18-20° C. over a period of 3 h. After addition, reaction mixture was then allowed to come to room temperature and stirred for 2 h. During this period, the dark viscous reaction mixture turned yellow in color with some amount of yellow solid precipitated. TLC revealed disappearance of starting material. Reaction mixture was poured over crushed ice water (1 kg) and stirred for 30 minutes. Pale yellow solid obtained was filtered, washed with water (100 ml) and dried under vacuum to yield 70 g of 4-methyl-3,5-dinitrobenzoic acid (83%). ¹H-NMR in DMSO-d₆δ ppm: 2.52 (3H, s, CH₃), 8.58 (2H, s, Ar—H).

Step 2: A solution of 4-methyl-3,5-dinitrobenzoic acid (70 g, 307 mmole) in methanol (750 ml) was warmed to 60° C. and a 30% solution of sodium hydrogen sulfide (133 ml, 539 mmole) was added slowly over a period of 30 minutes. The reaction mixture was refluxed for 3 h. The reaction mixture was evaporated to dryness and resulting residue was treated with water (500 ml). The mixture was acidified to pH 3 using dil. HCl. The precipitated product was extracted with ethyl acetate (3×250 ml) and the ethyl acetate layer was dried and evaporated to yield 50 g of 3-amino-4-methyl-5-nitrobenzoic acid as yellow solid (83%). ¹H-NMR in DMSO-d₆δ ppm: 2.14 (3H, s, CH₃), 5.85 (2H, bs, NH₂), 7.41 (1H, s, Ar—H), 7.56 (1H, s, Ar—H), 13.31 (1H, bs, COOH).

Step 3: A mixture of conc. sulfuric acid (367.5 ml) and water (117.6 ml) was heated to 90-100° C. and 3-amino-4-methyl-5-nitrobenzoic acid (29.4 g, 149 mmole) was added in small portion over a period of 30 minutes. Reaction mixture was then cooled to 0-5° C. and a solution of sodium nitrite (20.7 g, 300 mmole) in water (117.6 ml) was added over a period of 60 minutes. After addition, reaction mixture was stirred at 0-5° C. for another 30 minutes. The reaction mixture was then slowly allowed to come to 15-20° C. and then heated to 90-100° C. on a preheated oil-bath for 2-3 h. It was cooled to RT and water (630 ml) was added. Precipitated solid was filtered and dried under vacuum to yield 15 g of 3-hydroxy-4-methyl-5-nitrobenzoic acid as buff colored solid (51%). ¹H-NMR in DMSO-d₆δ ppm: 2.29 (3H, s, CH₃), 7.65 (1H, s, Ar—H), 7.78 (1H, s, Ar—H), 10.77 (1H, s, OH).

Step 4: At 0-5° C., to a solution of 3-hydroxy-4-methyl-5-nitrobenzoic acid (28 g, 141 mmole) in methanol (280 ml), thionyl chloride (15.5 ml, 212 mmole) was added dropwise over a period of 30 minutes. After the addition, the reaction mixture was brought to room temperature and then refluxed for 4 h. The reaction mixture was concentrated under vacuum. The resulting solid residue was dissolved in ethyl acetate (500 ml) and washed with sodium bicarbonate solution. Ethyl acetate extract was dried over Na₂SO₄and concentrated under vacuum to yield 30 g of methyl 3-hydroxy-4-methyl-5-nitrobenzoate as colorless solid (quantitative). The crude product was used as such for the next step without characterization.

Step 5: To a mixture of methyl 3-hydroxy-4-methyl-5-nitrobenzoate (30 g, 141 mmole), and K₂CO₃(38.9 g; 282 mmole) in acetone (300 ml) under inert atmosphere (nitrogen) was added methyl iodide (22.2 g, 156 mmole) at ambient temperature. The mixture was stirred overnight at the same temperature. The reaction mixture was filtered and acetone was removed on a rotavap. The resulting residue was dissolved in ethyl acetate (500 ml) and washed with dil. HCl. The organic phase was dried over Na₂SO₄and concentrated under vacuum to yield 30 g of methyl 3-methoxy-4-methyl-5-nitrobenzoate as off-white solid (94%). ¹H NMR in DMSO-d₆δ ppm: 2.33 (3H, s, CH₃), 3.91 (3H, s, OCH₃), 3.97 (3H, s, OCH₃), 7.7 (1H, s, Ar—H), 7.96 (1H, s, Ar—H).

Step 6: To a solution of methyl 3-methoxy-4-methyl-5-nitrobenzoate (30 g, 132 mmole) in methanol (200 ml) was added at room temperature 1M NaOH solution (158 ml, 158 mmole) and stirred overnight. The reaction mixture was concentrated on a rotavap and the resulting mixture was cooled to 5-10° C. and acidified with dil HCl to pH 2. Precipitated solid was filtered, washed with water and partly dried under vacuum. Partially dried colorless 3-methoxy-4-methyl-5-nitrobenzoic acid, weighing 30 g was used as such for the next step. ¹H NMR in DMSO-d₆δ ppm: 2.32 (3H, s, CH3) 3.95 (3H, s, OCH3) 7.69 (1H, s, ArH) 7.93 (1H, s, ArH).

Step 7: To a solution of KMnO₄(44.56 g, 282 mmole) in water (675 ml) was added at room temperature 3-methoxy-4-methyl-5-nitrobenzoic acid (30 g, 141 mmole) and the mixture was heated to reflux. After 2 h and 4 h of refluxing another lot of KMnO4 44.56 g (282 mmole) and 22.28 g (141 mmole) were added respectively. After complete consumption of starting material, the reaction mixture was cooled to room temperature and filtered through celite. The filtrate was concentrated to ca. 200 ml on a rotavap. The concentrated aqueous mixture was cooled to 0-5° C. and acidified to pH 2 with conc. HCl. The precipitated solid was filtered and washed with water to yield 15 g of 2-methoxy-6-nitroterephthalic acid as a colorless solid after drying (44%). ¹H NMR in DMSO-d₆δ ppm: 3.9 (3H, s, OCH₃), 7.81 (1H, s, Ar—H), 8.06 (1H, s, Ar—H).

Step 8: To a solution of 2-methoxy-6-nitroterephthalic acid (5 g, 21 mmole) in methanol (280 ml) maintained at 0-5° C. was added dropwise thionyl chloride (2.4 ml, 32 mmole) over a period of 5 minutes. The mixture was slowly brought to ambient temperature and was then refluxed for 4 h. The reaction mixture was concentrated under vacuum. The resulting solid residue was dissolved in ethyl acetate (150 ml) and washed with saturated Na₂CO₃solution. Ethyl acetate extract was dried over Na₂SO₄and evaporated to yield 5 g of 2-methoxy-6-nitro-terephthalic acid 4-methyl ester as colorless solid (93%). ¹H NMR in DMSO-d₆δ ppm: 3.93 (3H, s, OCH₃), 3.97 (3H, s, OCH₃), 7.92 (1H, s, Ar—H) 8.17 (1H, s, Ar—H).

Step 9: To a solution of 2-methoxy-6-nitro-terephthalic acid 4-methyl ester (7 g, 27 mmole) in methanol (100 ml) was added between 0-5° C. an ethereal solution of diazomethane (100 ml) [prepared from 50% aq. KOH (45 ml) and N-nitroso methylurea (11.3 g, 108 mmole)] over a period of 30 minutes. The reaction mixture was stirred between 0-5° C. for 1 h and then allowed to come to room temperature. Excess of diazomethane was then quenched by adding acetic acid. The reaction mixture was dried over Na₂SO₄and concentrated to yield 7 g of dimethyl 2-methoxy-6-nitroterephthalate (96%) as a pale yellow solid. ¹H-NMR in CDCl₃δ ppm: 3.98 (3H, s, OCH₃), 3.99 (3H, s, OCH₃), 4.36 (3H, s, OCH₃), 7.88 (1H, d, J=1.2 Hz, Ar—H), 8.4 (1H, d, J=1.2 Hz, Ar—H).

Step 10: To a solution of dimethyl 2-methoxy-6-nitroterephthalate (9.45 g, 35 mmole) in toluene (400 ml) was added at room temperature iron (20 g, 357 mmole) and the mixture was heated to reflux. Under reflux condition, acetic acid (33 ml) was added slowly over a period of 1 h and refluxing continued for 2 h. Two more lots of acetic acid (33 ml each) were added after a gap of 2 h for complete conversion. The reaction mass was cooled to room temperature and filtered through celite. The filtrate was concentrated on a rotavap and the resulting residue was subjected to column chromatographic purification over silica gel to yield 4.3 g of dimethyl 2-amino-6-methoxyterephthalate as yellow solid (51%). ¹H-NMR in CDCl₃δ ppm: 3.86 (3H, s, OCH₃), 3.89 (3H, s, OCH₃), 3.9 (3H, s, OCH₃), 5.05 (2H, bs, NH₂), 6.86 (1H, d, J=1.2 Hz, Ar—H), 6.97 (1H, d, J=1.2 Hz, Ar—H).

### 75b) Dimethyl 2-isothiocyanato-6-methoxy-1,4-benzenedicarboxylate

Thiophosgene (0.169 mL, 2.2 mmol, 1.1 eq.) was slowly added to a stirred mixture of dimethyl 2-amino-6-methoxy-1,4-benzenedicarboxylate (75a, 478 mg, 2.0 mmol, 1 eq.) in sat sodium bicarbonate (20 mL) and chloroform (20 mL) and stirred at rt for 2 hours. The phases were separated and the aqueous was extracted with DCM (3×20 mL). The combined organics were dried over anhydrous sodium sulfate, filtered and concentrated to give the title product (557 mg, yield 99%, 95% pure) which was used without purification. LC/MS: M+1=282

### 75c) 3-(5,6-Dimethoxy-2-pyridinyl)-5-methoxy-4-oxo-2-thioxo-1,2,3,4-tetrahydro-7-quinazolinecarboxylic acid

A mixture of dimethyl 2-isothiocyanato-6-methoxy-1,4-benzenedicarboxylate (75b, 0.557 g, 1.98 mmol, 1 eq.) and 5,6-dimethoxy-2-pyridinamine (0.305 g, 1.98 mmol, 1.0 eq.) in THF (20 mL) was heated at 50° C. for 2 hours, then continued stirring overnight. After adding 10% sodium hydroxide solution, the mixture was monitored with LCMS, and stirred for two hours then concentrated to dryness. The residue in DMSO was added dropwise into icy water and the precipitate was collected by filtration to give the title compound (yield, 587 mg, 76%, 100% purity by LCMS) which was used in the next step. LC/MS: M+1=390

### 75d) N-(4-Chlorobenzyl)-3-(5,6-dimethoxy-2-pyridinyl)-5-methoxy-4-oxo-2-thioxo-1,2,3,4-tetrahydro-7-quinazolinecarboxamide

HATU (84 mg, 0.22 mmol, 1.1 eq.) was added to a stirred solution of crude 3-(5,6-dimethoxy)-2-pyridinyl)-5-methoxy-4-oxo-2-thioxo-1,2,3,4-tetrahydro-7-quinazolinecarboxylic acid (75c, 78 mg, 0.20 mmol), 4-chlorobenzylamine (28.4 mg, 0.20 mmol, 1.0 eq.) and TEA (0.084 mL, 0.6 mmol, 3 eq.) in dry DMF (3 mL). The mixture was stirred at rt overnight Then added dropwise into cold water (30 mL). The solid was filtered off, dissolved in DMSO, filtered and the clear solution purified on HPLC under neutral condition (15-75% gradient in 8 minutes). (Yield: 36 mg, 35%). LC/MS: M+1=513. ¹H NMR (400 MHz, DMSO-d₆) δ ppm 3.77 (s, 3H) 3.85 (s, 3 H) 3.90 (s, 3H) 4.50 (d, J=5.81 Hz, 2H) 6.96 (d, J=8.08 Hz, 1H) 7.36-7.48 (m, 7H) 9.36 (t, J=5.94 Hz, 1H) 13.01 (s, 1H)

### Example 76

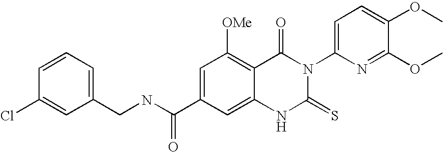

### N-(3-Chlorobenzyl)-3-(5,6-dimethoxy-2-pyridinyl)-5-methoxy-4-oxo-2-thioxo-1,2,3,4-tetrahydro-7-quinazolinecarboxamide

HATU (84 mg, 0.22 mmol, 1.1 eq.) was added to a stirred solution of the crude 3-(5,6-dimethoxy-2-pyridinyl)-5-methoxy-4-oxo-2-thioxo-1,2,3,4-tetrahydro-7-quinazolinecarboxylic acid (75c, 78 mg, 0.20 mmol), 3-chlorobenzylamine (28.4 mg, 0.20 mmol, 1.0 eq.) and TEA (0.084 mL, 0.6 mmol, 3 eq.) in dry DMF (3 mL). The mixture was stirred at rt overnight then added dropwise into cold water (30 mL). The solid was filtered off, dissolved in DMSO, filtered and the clear solution purified on HPLC under neutral condition (15-75% gradient in 8 minutes). (Yield, 55 mg 53%). LC/MS: M+1=513. ¹H NMR (400 MHz, DMSO-d₆δ ppm: 3.77 (s, 3H) 3.85 (s, 3H) 3.90 (s, 3H) 4.50 (d, J=5.81 Hz, 2H) 6.96 (d, J=8.08 Hz, 1H) 7.36-7.48 (m, 7H) 9.36 (t, J=5.94 Hz, 1H) 13.01 (s, 1H)

### Example 77

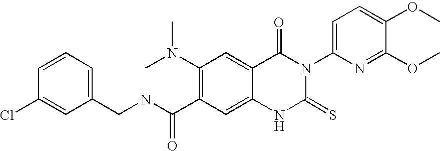

### N-(3-Chlorobenzyl-3-[5,6-dimethoxy)-2-pyridinyl]-6-(dimethylamino)-4-oxo-2-thioxo-1,2,3,4-tetrahydro-7-quinazolinecarboxamide

### 77a) dimethyl 2-amino-5-(dimethylamino)-1,4-benzenedicarboxylate

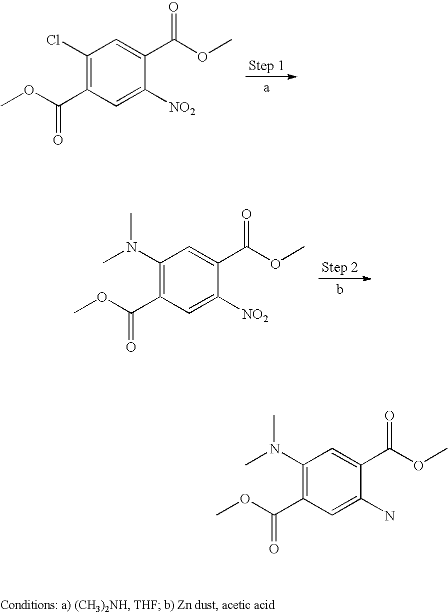

Step 1: To a tetrahydrofuran solution (2 mL) of dimethyl 2-chloro-5-nitro-1,4-benzenedicarboxylate (547 mg, 2 mmol) in a 5 mL microwave tube, was added dimethylamine (90 mg, 2.0 mmol) to give a yellow solution. The reaction was heated via Biotage Microwave reactor at 85° C. for 15 minutes and checked by LCMS. The reaction mixture was concentrated, diluted with ethyl acetate (75 mL), washed with water (10 mL), dried over anhydrous sodium sulfate, filtered and concentrated to give the title product (564 mg, 100%) which was used without purification. LC/MS: M+1=283

Step 2: In a 25 mL round-bottomed flask was added the dimethyl ester (13a, 564 mg, 2 mmol), zinc dust (1.3 g, 20 mmol) and acetic acid (10 mL) to give a yellow suspension. The reaction was stirred for 30 minutes at which time LCMS (m+1=252.8) showed that the reaction was complete. The reaction mixture was diluted with ethyl acetate (25 mL), filtered through celite and rinsed with ethyl acetate (10 mL). The filtrate was concentrated to dryness, and then characterized by LCMS and NMR. Yield: 505 mg, 100%. LC/MS: M+1=253

### 77b) Dimethyl 2-dimethylamino-5-isothiocyanato-1,4-benzenedicarboxylate

Thiophosgene (0.169 mL, 2.2 mmol, 1.1 eq.) was slowly added to a stirred solution of dimethyl 2-amino-5-dimethylamino-1,4-benzenedicarboxylate (77a, 505 mg, 2.0 mmol, 1 eq.) in saturated sodium bicarbonate solution (20 mL) and chloroform (20 mL) and the mixture was stirred at rt for 2.5 hours. The phases were separated and the aqueous was extracted with DCM (3×25 mL). The combined organics were dried over anhydrous sodium sulfate, filtered and concentrated to give the title product (0.505 g, 86%) which was used without purification. LC/MS: M+1=295

### 77c) 3-(5,6-Dimethoxy-2-pyridinyl)-6-dimethylamino-4-oxo-2-thioxo-1,2,3,4-tetrahydro-7-quinazolinecarboxylic acid

A mixture of dimethyl 2-dimethylamino-5-isothiocyanato-1,4-benzenedicarboxylate (77b, 0.505 g, 1.72 mmol, 1 eq.) and 5,6-dimethoxy-2-pyridinamine (0.265 g, 1.72 mmol, 1.0 eq.) in THF (20 mL) was heated at 50° C. for overnight. After adding 10% sodium hydroxide solution, the mixture was monitored with LCMS, and stirred for two hours then concentrated to dryness. The residue in DMSO was dropped slowly into icy water and the precipitate collected by filtration. The title product (408 mg, 59%) was obtained with 100% purity and used in the next step. LC/MS: M+1=403

### 77d) N-(3-Chlorobenzyl)-3-(5,6-dimethyloxy-2-pyridinyl)-6-dimethylamino-4-oxo-2-thioxo-1,2,3,4-tetrahydro-7-quinazolinecarboxamide

3-Chlorobenzylamine (53 mg, 0.373 mmol, 1.0 eq.), HATU (184 mg, 0.48 mmol, 1.3 eq.) and TEA (57 mg, 0.56 mmol, 1.5 eq.) were added to a stirred solution of 3-(5,6-dimethoxy-2-pyridinyl)-6-dimethylamino-4-oxo-2-thioxo-1,2,3,4-tetrahydro-7-quinazolinecarboxylic acid (77c, 150 mg, 0.373 mmol) in dry DMF (1.5 mL). The mixture was stirred at rt overnight then added dropwise into cold water (30 mL). The solid was filtered off, dissolved in DMSO, filtrated and purified on Gilson HPLC (15-75% gradient in 8 minutes) to give the title compound. (yield: 39 mg, 20%). LC/MS: M+1=527. ¹H NMR (400 MHz, DMSO-d₆) δ ppm 2.71 (s, 6H) 3.78 (s, 3H) 3.86 (s, 3H) 4.51 (d, J=6.06 Hz, 2H) 7.00 (d, J=8.08 Hz, 1H) 7.36 (d, J=1.26 Hz, 1H) 7.34 (t, J=2.15 Hz, 1H) 7.39-7.49 (m, 4H) 7.53 (s, 1H) 7.57 (s, 1H) 9.42 (s, 1H) 13.09 (s, 1H)

### Example 78

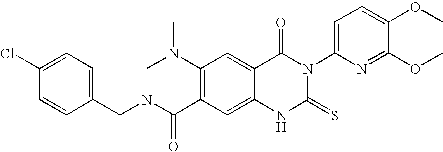

### N-(4-Chlorobenzyl)-3-(5,6-dimethoxy-2-pyridinyl)-6-dimethylamino-4-oxo-2-thioxo-1,2,3,4-tetrahydro-7-quinazolinecarboxamide

HATU (123 mg, 0.323 mmol, 1.3 eq.) was added to a stirred solution of the crude 3-(5,6-dimethoxy-2-pyridinyl)-6-dimethylamino-4-oxo-2-thioxo-1,2,3,4-tetrahydro-7-quinazolinecarboxylic (77c, 100 mg, 0.248 mmol), 4-chlorobenzylamine (35.2 mg, 0.248 mmol, 1.0 eq.) and TEA (0.052 mL, 0.373 mmol, 1.5 eq.) in dry DMF (2 mL). The mixture was stirred at rt overnight then added dropwise into cold water (35 mL). The solid was filtered off, dissolved in DMSO, filtered and the clear solution was purified on HPLC under neutral condition (15-75% gradient in 8 minutes). (Yield: 26.5 mg, 20.3%). LC/MS: M+1=527. ¹H NMR (400 MHz, DMSO-d₆) δ ppm 2.70 (s, 6H) 3.78 (s, 3H) 3.86 (s, 3H) 4.48 (d, J=6.06 Hz, 2H) 6.98 (s, 1H) 7.37-7.49 (m, 3H) 7.43 (d, J=3.03 Hz, 2H) 7.51 (s, 1H) 7.59 (s, 1H) 9.44 (s, 1H) 13.08 (s, 1H)

### Example 79

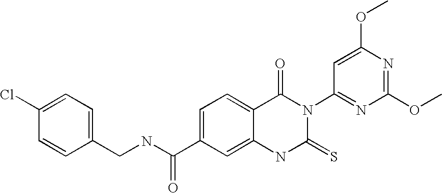

### N-(4-Chlorobenzyl)-3-[2,6-dimethoxy-4-pyrimidinyl]-4-oxo-2-thioxo-1,2,3,4-tetrahydro-7-quinazolinecarboxamide

### 79a) 3-[2,6-Dimethoxy-4-pyrimidinyl]-4-oxo-2-thioxo-1,2,3,4-tetrahydro-7-quinazolinecarboxylic acid

To a solution of dimethyl 2-isothiocyanato-1,4-benzenedicarboxylate (1a, 100 mg, 0.398 mmol, 1 eq.) in dry DMSO (1 ml) was added 2,6-dimethoxy-4-pyrimidinamine (62 mg, 0.398 mmol, 1 eq.). The resultant translucent brown mixture was heated to 65° C. overnight with stirring. Upon the addition of aqueous sodium hydroxide (NaOH, 0.6 ml, 10N) the solution became translucent green and within 5 minutes changed to cloudy yellow. The mixture was diluted with water (2 ml) and adjusted to <pH 2 with concentrated HCl. The solution became colorless before a pale yellow solid precipitated out. The solid was collected by vacuum filtration, washed with water (14 ml) and dried in-vacuo to yield the title compound in 99% purity. Yield: not calculated. LC/MS: M+1=361

### 79b) N-(4-Chlorobenzyl)-3-[2,6-dimethoxy-4-pyrimidinyl]-4-oxo-2-thioxo-1,2,3,4-tetrahydro-7-quinazolinecarboxamide

A stirred solution of 3-[2,6-dimethoxy-4-pyrimidinyl]-4-oxo-2-thioxo-1,2,3,4-tetrahydro-7-quinazolinecarboxylic acid (79a, 143 mg, 0.398 mmol, 1 eq.) in dry DMF (1 ml) were added 4-chlorobenzylamine (48.4 μl, 0.438 mmol, 1.1 eq.), DIEA (76 μl 0.438 mmol, 1.1 eq.), and HATU (166 mg, 0.438 mmol, 1.1 eq.) in order. The mixture was stirred at rt less than 1 hour then poured into ice water (6 ml) which became cloudy. The mixture was then diluted with more water (4 ml), shaken and left to stand until product precipitated out. The solid was collected by vacuum filtration, washed with water and purified by HPLC using acidic conditions (35-65% gradient over 8 minutes) to yield the title compound as an off white solid. Yield: 23 mg, 12%; LC/MS: M+1=484 ¹H-NMR (400 MHz, DMSO-d₆) δ ppm 3.90 (s, 3H), 3.99 (s, 3H), 4.48 (d, J=5.81 Hz, 2H), 6.81 (s, 1H), 7.33-7.45 (m, 4H), 7.79 (dd, J=8.34, 1.26 Hz, 1H), 7.89 (d, J=1.01 Hz, 1H), 8.06 (d, J=8.34 Hz, 1H), 9.41 (t, J=5.94 Hz, 1H), 13.36 (s, 1H)

### Example 80

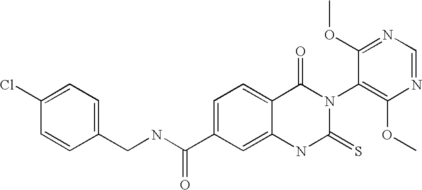

### N-(4-Chlorobenzyl)-3-[4,6-dimethoxy-5-pyrimidinyl]-4-oxo-2-thioxo-1,2,3,4-tetrahydro-7-quinazolinecarboxamide

### 80a) 3-[4,6-Dimethoxy-5-pyrimidinyl]-4-oxo-2-thioxo-1,2,3,4-tetrahydro-7-quinazolinecarboxylic acid

To a rapidly stirred solution of 4,6-dimethoxy-5-pyrimidinamine (185 mg, 1.19 mmol, 1 eq.) in dry DMSO (1 ml) was added sodium hydride (NaH, 2.8 mg, 1 eq.) as a dry powder. After the closed system stirred at rt 10 minutes a solution of dimethyl 2-isothiocyanato-1,4-benzenedicarboxylate (1a, 300 mg, 1.19 mmol, 1 eq.) in dry DMSO (1.5 ml) was added dropwise and stirring continued at rt overnight. LCMS gave confirmation that starting material had been consumed yielding a mixture of desired product and a thiourea-intermediate. The pH was raised with sodium hydroxide (1 ml, 10N). After stirring at rt for 10 minutes the pH was slowly lowered with concentrated hydrochloric acid (1.1 ml). The reaction was poured into water, shaken and left standing at rt until the product precipitated out. The solid was collected with vacuum filtration, washed with water and dried in-vacuo to yield the title compound as an off white powder. Yield: not calculated. LC/MS: M+1=361

### 80b) (4-Chlorobenzyl)-3-[4,6-dimethoxy-5-pyrimidinyl]-4-oxo-2-thioxo-1,2,3,4-tetrahydro-7-quinazolinecarboxamide

To a stirred solution of 3-[4,6-dimethoxy-5-pyrimidinyl]-4-oxo-2-thioxo-1,2,3,4-tetrahydro-7-quinazolinecarboxylic acid (80a, 123 mg, 0.342 mmol, 1 eq.) in dry DMF (3 ml) were added DIEA (65 μl, 0.376 mmol, 1.1 eq.), and HATU (143 mg, 0.376 mmol, 1.1 eq.) After stirring at rt for 20 minutes, 4-chlorobenzylamine (48.4 μl, 0.438 mmol, 1.1 eq.) was added. The mixture was stirred at rt for 1.5 hours then poured into ice water (6 ml) and stirred until a solid precipitated out. The solid was collected by vacuum filtration, washed with water and purified by HPLC using acidic conditions (40-55% gradient over 8 minutes) to yield the title compound as an off white solid. Yield: 6 mg, 3.6%; LC/MS: M+1=484; ¹H NMR (400 MHz, DMSO-d₆) δ ppm 3.90 (d, J=1.01 Hz, 6H), 4.49 (d, J=6.06 Hz, 2H), 7.39 (dd, J=11.24, 0.63 Hz, 4H), 7.81 (d, J=8.34 Hz, 1H), 7.90 (s, 1H), 8.06 (d, J=8.34 Hz, 1H), 8.61 (d, J=1.01 Hz, 1H), 9.41 (t, J=5.81 Hz, 1H), 13.50 (s, 1H)

### Example 81

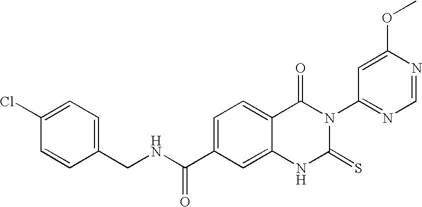

### N-(4-Chlorobenzyl)-3-[6-methoxy-4-pyrimidinyl]-4-oxo-2-thioxo-1,2,3,4-tetrahydro-7-quinazolinecarboxamide

### 81a) 3-[6-Methoxy-4-pyrimidinyl]-4-oxo-2-thioxo-1,2,3,4-tetrahydro-7-quinazolinecarboxylic acid

To solution of 6-methoxy-4-pyrimidinamine (185 mg, 1.19 mmol, 1 eq.) in dry DMF (1 ml) was added a solution of dimethyl 2-isothiocyanato-1,4-benzenedicarboxylate (1a, 200 mg, 0.796 mmol, 1 eq.) in dry DMSO (2 ml) and the solution was heated to 65° C. 2.5 days with stirring. The pH was raised with aqueous sodium hydroxide (2 ml, 10M) and stirred for 2 minutes before the pH was slowly lowered with concentrated hydrochloric acid (2.2 ml). The reaction was poured into 30 ml of water and stirred for 10 minutes until the product precipitated out. The solid was collected with vacuum filtration, washed with water and dried in-vacuo to yield the title compound as an off white powder. Yield: not calculated. LC/MS: M+1=331

### 81b) N-(4-Chlorobenzyl)-3-[6-methoxy-4-pyrimidinyl]-4-oxo-2-thioxo-1,2,3,4-tetrahydro-7-quinazolinecarboxamide

To a stirred solution of 3-[6-methoxy-4-pyrimidinyl]-4-oxo-2-thioxo-1,2,3,4-tetrahydro-7-quinazolinecarboxylic acid (81a, 236 mg, 0.796 mmol, 1 eq.) in dry DMF (8 ml) were added DIEA (152 μl, 0.876 mmol, 1.1 eq.), and HATU (333 mg, 0.876 mmol, 1.1 eq.) After stirring at rt for 10 minutes, 4-chlorobenzylamine (107 μl, 0.876 mmol, 1.1 eq.) was added. The mixture was stirred at rt for 1 hour then poured into ice water (6 ml) and stirred until a solid precipitated. The solid was collected by vacuum filtration, washed with water and purified by HPLC using acidic conditions (30-65% gradient over 8 minutes) to yield the title compound as an off white solid. Yield: 20 mg, 36.1%; LC/MS: M+1=454; ¹H NMR (400 MHz, DMSO-d₆) δ ppm 4.02 (s, 3H), 4.49 (d, J=5.81 Hz, 2H), 7.21 (d, J=1.01 Hz, 1H), 7.33-7.45 (m, 4H), 7.79 (dd, J=8.34, 1.52 Hz, 1H), 7.89 (d, J=1.26 Hz, 1H), 8.07 (d, J=8.34 Hz, 1H), 8.92 (d, J=1.01 Hz, 1H), 9.42 (t, J=6.06 Hz, 1H), 13.39 (s, 1H)

### Example 82

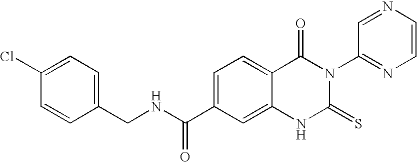

### N-(4-Chlorobenzyl)-4-oxo-3-(2-pyrazinyl)-2-thioxo-1,2,3,4-tetrahydro-7-quinazolinecarboxamide

### 82a) Methyl 2-amino-4-(4-chlorobenzylaminocarbonyl)benzoate

To a stirred suspension of 3-amino-4-(methoxycarbonyl)benzoic acid (20 g, 0.10 mol, 1 eq.) in DMF (205 mL) was added HATU (43 g, 0.11 mol, 1.1 eq.) and DIEA (19.6 mL, 0.11 mol, 1.1 eq.) and the mixture stirred at rt for 10 min until all dissolved. To this stirred solution was added a 9M solution of 4-chlorobenzylamine (13.7 ml, 0.11 mol, 1.1 eq.) in DMF (12 ml) via syringe. The solution was stirred at rt overnight, and after confirmation by LCMS that the reaction was complete the product was precipitated from ice water (2 L, crush ice, DI water). The product was collected by vacuum filtration and dried in a vacuum oven at 30° C. for 48 hours to yield the title product as a pale cream powder which was used without further purification. Yield: 30 g, 94%; LC/MS: M+1=319; ¹H NMR (400 MHz, DMSO-d₆) δ ppm 3.81 (s, 3H), 4.42 (d, J=6.06 Hz, 2H), 6.80 (s, 2H), 6.96 (dd, J=8.34, 1.77 Hz, 1H), 7.26 (d, J=1.52 Hz, 1H), 7.29-7.36 (m, 2H), 7.36-7.43 (m, 2H), 7.76 (d, J=8.59 Hz, 1H), 9.06 (t, 1H)

### 82b) Methyl 4-(4-chlorobenzylaminocarbonyl)-2-isothiocyanatobenzoate

To a rapidly stirred solution of methyl 2-amino-4-(4-chlorobenzylaminocarbonyl)benzoate (82a, 500 mg, 1.57 mmol, 1 eq) in sat sodium bicarbonate (8 ml) and chloroform (8 ml), thiophosgene was added dropwise (0.145 ml, 1.88 mmol, 1.2 eq). After stirring at rt overnight the product was extracted with DCM (3×15 ml) and the combined organic layers were dried over anhydrous sodium sulfate, filtered and concentrated then dried in vacuo. The resulting light tan solid was used without further purification, 99% purity. Yield: 513 mg, 91%; LC/MS: M+1=361

### 82c) N-(4-Chlorobenzyl)-4-oxo-3-(2-pyrazinyl)-2-thioxo-1,2,3,4-tetrahydro-7-quinazolinecarboxamide

To a rapidly stirred solution of methyl 4-(4-chlorobenzylaminocarbonyl)-2-isothiocyanatobenzoate (82b, 50 mg, 0.138 mmol, 1 eq) in DMSO (0.5 ml) under an inert atmosphere was, added sodium hydride as a dry powder (3.3 mg, 0.138 mmol, 1 eq). After the resulting mixture was stirred for 5 min at rt, a DMSO solution (0.5 ml) of 2-pyrazinamine (13 mg, 0.138 mmol, 1 eq) was added dropise. The reaction mixture was stirred at rt overnight then precipitated with hydrochloric acid (1 ml, 1M). The solid was recrystallisation from methanol (MeOH, 5 ml) then purified by HPLC under acidic conditions (35-60% gradient over 8 minutes) to produce the title compound as a pale yellow solid. Yield: 12 mg, 20.6%; LC/MS: M+1=424; ¹H-NMR (400 MHz, DMSO-d₆) δ ppm 4.49 (d, J=5.81 Hz, 2H), 7.34-7.48 (m, 4H), 7.81 (dd, J=8.34, 1.52 Hz, 1H), 7.92 (d, J=1.01 Hz, 1H), 8.09 (d, J=8.08 Hz, 1H), 8.75 (s, 2H), 8.84 (s, 1H), 9.43 (t, J=5.94 Hz, 1H), 13.48 (br. s., 1H)

### Example 83

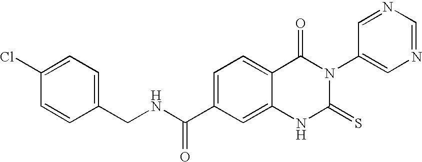

### N-(4-Chlorobenzyl)-4-oxo-3-(5-pyrimidinyl)-2-thioxo-1,2,3,4-tetrahydro-7-quinazolinecarboxamide

To a rapidly stirred solution of methyl 4-(4-chlorobenzylaminocarbonyl)-2-isothiocyanatobenzoate (82b, 100 mg, 0.277 mmol, 1 eq) in DMSO (1 ml) under an inert atmosphere, was added sodium hydride as a dry powder (6.6 mg, 0.277 mmol, 1 eq). After the resulting mixture was stirred for 5 minutes at rt, a DMSO solution (1 ml) of 5-pyrimidinamine (26.3 mg, 0.277 mmol, 1 eq) was added dropwise. The reaction mixture was stirred at rt overnight then precipitated with aqueous hydrochloric acid (1 ml, 1M). The solid was purified by HPLC under acidic conditions (35-60% gradient over 8 minutes) to produce the title compound as a white solid. Yield: 34 mg, 30.6%; LC/MS: M+1=424; ¹H-NMR (400 MHz, DMSO-d₆) δ ppm 4.49 (d, J=6.06 Hz, 2H), 7.33-7.47 (m, 4H), 7.82 (dd, J=8.34, 1.52 Hz, 1H), 7.92 (d, J=1.26 Hz, 1H), 8.09 (d, J=8.08 Hz, 1H), 8.75 (s, 2H), 8.84 (s, 1H), 9.43 (t, J=5.94 Hz, 1H), 13.48 (s, 1H)

### Example 84

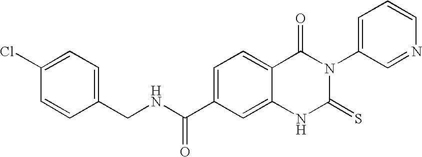

### N-(4-Chlorobenzyl)-4-oxo-3-(3-pyridinyl)-2-thioxo-1,2,3,4-tetrahydro-7-quinazolinecarboxamide

A mixture of methyl 4-(4-chlorobenzylaminocarbonyl)-2-isothiocyanatobenzoate (82b, 60 mg, 0.166 mmol, 1.0 eq.) and 3-pyridinamine (0.031 g, 0.33 mmol, 2.0 eq.) in THF (1.5 mL) was heated at 55° C. for two hours. The cooled reaction was diluted with methanol (1 mL), heated to boiling and cooled to precipitate the product which was filtered off and washed with cold methanol. Yield: 20.1 mg, 100% purity. Yield: 29%; LC/MS: M+1=423; ¹H NMR (400 MHz, DMSO-d₆) δ ppm 4.49 (s, 2H) 7.40 (m, 4H) 7.55 (s, 1H) 7.79 (s, 1H) 7.90 (s, 1H) 8.05 (s, 1H) 8.51 (s, 1H) 8.60 (s, 1H) 9.40 (s, 1H) 13.31 (s, 1H)

### Example 85

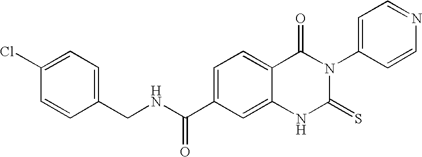

### N-(4-Chlorobenzyl)-4-oxo-3-(4-pyridinyl)-2-thioxo-1,2,3,4-tetrahydro-7-quinazolinecarboxamide

A mixture of methyl 4-(4-chlorobenzylaminocarbonyl)-2-isothiocyanatobenzoate (82b, 60 mg, 0.166 mmol, 1 eq.) and 4-pyridinamine (0.031 g, 0.33 mmol, 2.0 eq.) in THF (1.5 mL) was heated at 55° C. for two hours, and then heated at 65° C. overnight. The cooled reaction was diluted with methanol (1 mL), heated to boiling and cooled to precipitate the product which was filtered off and washed with cold methanol. Yield: 30.1 mg, 43%; 100% purity. LC/MS: M+1=423; 1H NMR (400 MHz, DMSO-d₆) δ ppm 4.47 (s, 2H) 7.40 (m, 4H) 7.56 (s, 2H) 7.78 (s, 1H) 7.89 (s, 1H) 8.06 (s, 1H) 8.79 (m, 2H) 9.40 (s, 1H) 13.37 (s, 1H)

### Example 86

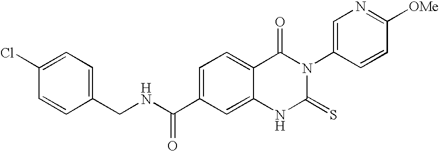

### N-(4-Chlorobenzyl)-3-[6-methoxy-3-pyridinyl]-4-oxo-2-thioxo-1,2,3,4-tetrahydro-7-quinazolinecarboxamide

The mixture of methyl 4-(4-chlorobenzylaminocarbonyl)-2-isothiocyanatobenzoate (82b, 72 mg, 0.2 mmol, 1 eq.) and 6-methoxy-3-pyridinamine (24.8 mg, 0.2 mmol, 1.0 eq.) in THF (2 mL) was heated at 55° C. for two hours. The cooled mixture was purified on HPLC under neutral condition (15-75% gradient in 8 minutes). Yield: 20.6 mg, 23%, 100% purity; LC/MS: M+1=453; 1H NMR (400 MHz, DMSO-d₆) δ ppm 3.90 (s, 3H) 4.48 (s, 2H) 6.95 (s, 1H) 7.40 (m, 4H) 7.68 (s, 1H) 7.77 (s, 1H) 7.89 (s, 1H) 8.06 (s, 2H) 9.40 (s, 1H) 13.27 (s, 1H)

### Example 87

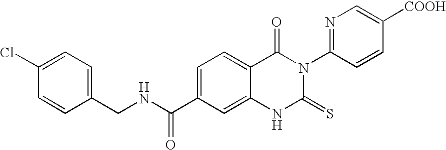

### 6-[7-(4-Chlorobenzylaminocarbonyl)-4-oxo-2-thioxo-1,4-dihydro-3(2H)-quinazolinyl]-3-pyridinecarboxylic acid

A mixture of methyl 4-(4-chlorobenzylaminocarbonyl)-2-isothiocyanatobenzoate (82b, 0.237 g, 0.66 mmol, 1 eq.) and methyl 6-amino-3-pyridinecarboxylate (0.100 g, 0.66 mmol, 1.0 eq.) in DMF (1.5 mL) was heated at 60° C. overnight with stirring. Sodium hydroxide solution (2N) was added and ester hydrolysis was monitored with LCMS. After stirring for two hours, the reaction was diluted with DMSO (1 mL) and purified on HPLC (15-75% gradient in 8 minutes) under acidic condition to give 55.1 mg of the title compound. Yield: 55.1 mg, 18%; LC/MS: M+1=467; 1H NMR (400 MHz, DMSO-d₆) δ ppm 4.48 (s, 2H) 7.40 (s, 4H) 7.70 (s, 1H) 7.80 (s, 1H) 7.92 (s, 1H) 8.07 (s, 1H) 8.47 (s, 1H) 9.09 (s, 1H) 9.42 (s, 1H) 13.38 (s, 1H)

### Example 88

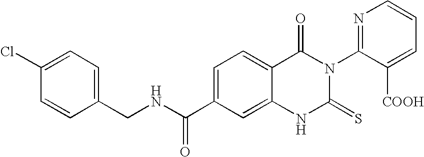

### 2-[7-(4-Chlorobenzylaminocarbonyl)-4-oxo-2-thioxo-1,4-dihydro-3(2H)-quinazolinyl]-3-pyridinecarboxylic acid

A mixture of methyl 4-(4-chlorobenzylaminocarbonyl)-2-isothiocyanatobenzoate (82b, 108 mg, 0.3 mmol, 1 eq.) and 2-amino-3-pyridinecarboxylic acid (42 mg, 0.3 mmol, 1.0 eq.) in DMF (2 mL) was heated at 120° C. with stirring in a microwave reactor. This reaction mixture was diluted with DMSO (1 mL), filtered and purified on (HPLC 15-75% gradient in 8 minutes) under acidic condition to give the title product. Yield: 20.2 mg, 14%, 97% purity; LC/MS: M+1=466.8; 1H NMR (400 MHz, DMSO-d₆) δ ppm 4.49 (s, 2H) 7.39 (m, 4H) 7.69 (s, 1H) 7.80 (s, 1H) 7.91 (s, 1H) 8.09 (s, 1H) 8.50 (s, 1H) 8.81 (s, 1H) 9.42 (m, 1H) 13.35 (s, 1H) 13.35 (s, 1H)

### Example 89

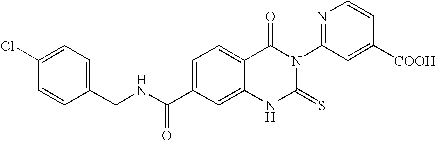

### 2-[7-(4-Chlorobenzylaminocarbonyl)-4-oxo-2-thioxo-1,4-dihydro-3(2H)-quinazolinyl]-4-pyridinecarboxylic acid

A mixture of methyl 4-(4-chlorobenzylaminocarbonyl)-2-isothiocyanatobenzoate (82b, 180 mg, 0.5 mmol, 1 eq.) and ethyl 2-amino-4-pyridinecarboxylate (83 mg, 0.5 mmol, 1.0 eq.) in DMF (2 mL) was heated at 100° C. overnight with stirring. Sodium hydroxide solution (2N) was added and ester hydrolysis was monitored with LCMS. After stirring for two hours, the reaction was diluted with DMSO (1 mL) and purified on HPLC (15-75% gradient in 8 minutes) under acidic condition to give the title product. Yield: 20.1 mg, 9%; LC/MS: M+1=466.8; 1H NMR (400 MHz, DMSO-d₆) δ ppm 4.51 (s, 2H) 7.39 (m, 4H) 7.76 (s, 1H) 7.90 (s, 2H) 7.99 (s, 1H) 8.06 (s, 1H) 8.76 (s, 1H) 9.41 (t, 1H) 13.33 (s, 1H)

### Example 90

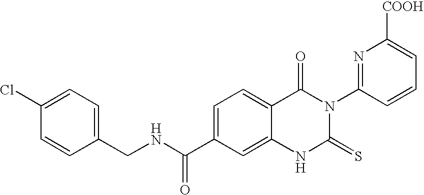

### 6-[7-({(4-Chlorobenzyl)amino}carbonyl)-4-oxo-2-thioxo-1,4-dihydro-3(2H)-quinazolinyl]-2-pyridinecarboxylic acid

A mixture of methyl 4-(4-chlorobenzylaminocarbonyl)-2-isothiocyanatobenzoate (82b, 180 mg, 0.5 mmol, 1 eq.) and methyl 6-amino-2-pyridinecarboxylate (76 mg, 0.5 mmol, 1.0 eq.) in DMF (2 mL) was heated at 100° C. overnight with stirring. Sodium hydroxide solution (2N) was added and ester hydrolysis was monitored with LCMS. After stirring for two hours at 60° C., the reaction was diluted with DMSO (1 mL) and purified on HPLC (30-60% gradient in 8 minutes) under acidic condition to give the title product. Yield: 20.1 mg, 9%; LC/MS: M+1=466.8; 1H NMR (400 MHz, DMSO-d₆) δ ppm 4.48 (s, 2H) 7.39 (m, 4H) 7.80 (s, 2H) 7.90 (s, 1H) 8.05 (s, 1H) 8.18 (s, 2H) 9.44 (t, 1H) 13.33 (s, 1H)

### Example 91

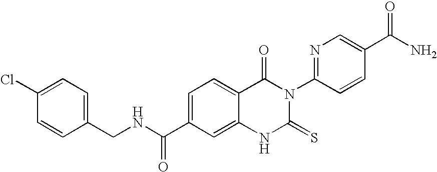

### 3-[5-(Aminocarbonyl)-2-pyridinyl]-N-(4-chlorobenzyl)-4-oxo-2-thioxo-1,2,3,4-tetrahydro-7-quinazolinecarboxamide

A mixture of methyl 4-(4-chlorobenzyl)aminocarbonyl)-2-isothiocyanatobenzoate (82b, 144 mg, 0.4 mmol, 1 eq.) and 6-amino-3-pyridinecarboxamide (54 mg, 0.4 mmol, 1.0 eq.) in DMSO (2 mL) was heated at 100° C. with stirring in a microwave reactor. The reaction mixture was diluted with DMSO (6 mL), filtered and purified on HPLC (10-90% gradient system in 8 minutes) under acidic condition to give the title product. Yield: 23.1 mg, 12%; LC/MS: M+1=466; 1H NMR (400 MHz, DMSO-d₆) δ ppm 3.81 (s, 2H) 4.48 (d, J=6.32 Hz, 2H) 7.35-7.42 (m, 4H) 7.86 (dd, J=8.34, 1.52 Hz, 1H) 7.97 (d, J=8.34 Hz, 1H) 8.26 (dd, J=8.72, 2.40 Hz, 1H) 8.45 (d, J=1.52 Hz, 1H) 8.80 (s, 1H) 9.27 (t, 1H) 11.33 (s, 1H) 13.92 (s, 1H)

### Example 92

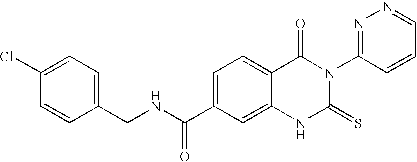

### N-(4-Chlorobenzyl)-4-oxo-3-(3-pyridazinyl)-2-thioxo-1,2,3,4-tetrahydro-7-quinazolinecarboxamide

A mixture of methyl 4-(4-chlorobenzylaminocarbonyl)-2-isothiocyanatobenzoate (82b, 144 mg, 0.4 mmol, 1 eq.) and 3-pyridazinamine (38 mg, 0.4 mmol, 1.0 eq.) in DMSO (2 mL) was heated overnight at 60° C. with stirring. This mixture was diluted with DMSO (1 mL), filtered and purified on HPLC under acidic condition to give the title product. Yield: 20.6 mg, 12.1%; LC/MS: M+1=424; 1H NMR (400 MHz, DMSO-d₆) δ ppm 4.50 (s, 2H) 7.40 (s, 4H) 7.82 (s, 1H) 7.95 (s, 3H) 8.09 (s, 1H) 9.33 (s, 1H) 9.39-9.45 (m, 1H) 13.47 (s, 1H)

### Example 93

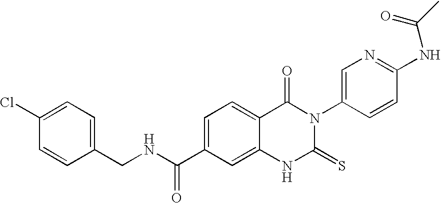

### 3-[6-(Acetylamino)-3-pyridinyl]-N-(4-chlorobenzyl)-4-oxo-2-thioxo-1,2,3,4-tetrahydro-7-quinazolinecarboxamide

A mixture of methyl 4-(4-chlorobenzylaminocarbonyl)-2-isothiocyanatobenzoate (82b, 144 mg, 0.4 mmol, 1 eq.) and N-(5-amino-2-pyridinyl)acetamide (60.4 mg, 0.4 mmol, 1.0 eq.) in DMSO (2 mL) was heated at 60° C. with stirring for 3 hours. The reaction mixture was diluted with DMSO (1 mL), filtered and purified on HPLC (10-90% gradient system in 8 minutes) under acidic condition to give the title product. Yield: 26 mg, 11%; LC/MS: M+1=480; 1H NMR (400 MHz, DMSO-d₆) δ ppm 4.49 (s, 2H) 7.40 (s, 4H) 7.72-7.79 (m, 2H) 7.90 (s, 1H) 8.08 (s, 1H) 8.17 (s, 1H) 8.23 (s, 1H) 9.37-9.41 (m, 1H) 10.67 (s, 1H) 13.30 (s, 1H)

### Example 94

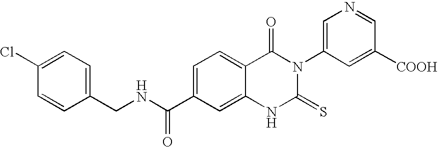

### 5-[7-(4-Chlorobenzylaminocarbonyl)-4-oxo-2-thioxo-1,4-dihydro-3(2H)-quinazolinyl]-3-pyridinecarboxylic acid

A mixture of methyl 4-(4-chlorobenzylaminocarbonyl)-2-isothiocyanatobenzoate (82b, 144 mg, 0.4 mmol, 1 eq.) and 5-amino-3-pyridinecarboxylic acid (55.2 mg, 0.4 mmol, 1.0 eq.) in DMSO (2 mL) was heated overnight at 100° C. with stirring. The reaction mixture was diluted with DMSO (3 mL), filtere and purified on HPLC (10-90% gradient system in 8 minutes) under acidic condition to give the title product. Yield: 12.8 mg, 7%; LC/MS: M+1=466.8; 1H NMR (400 MHz, DMSO-d₆) δ ppm 4.49 (s, 2H) 7.40 (s, 4H) 7.79 (s, 1H) 7.91 (s, 1H) 8.07 (s, 1H) 8.33 (s, 1H) 8.76 (s, 1H) 9.10 (s, 1H) 9.41 (s, 1H) 13.39 (s, 1H)

### Example 95

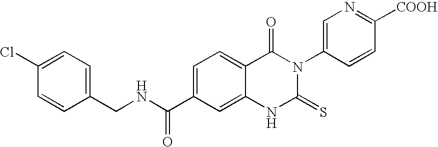

### 5-[7-(4-Chlorobenzylaminocarbonyl)-4-oxo-2-thioxo-1,4-dihydro-3(2H)-quinazolinyl]-2-pyridinecarboxylic acid

A mixture of methyl 4-(4-chlorobenzylaminocarbonyl)-2-isothiocyanatobenzoate (82b, 144 mg, 0.4 mmol, 1 eq.) and 5-amino-2-pyridinecarboxylic acid (55.2 mg, 0.4 mmol, 1.0 eq.) in DMSO (2 mL) was heated overnight at 100° C. with stirring. The reaction mixture was diluted with DMSO (3 mL), filtered and purified on HPLC (10-90% gradient system in 8 minutes) under acidic condition to give the title product. Yield: 22.5 mg, 9%; LC/MS: M+1=466.8; 1H NMR (400 MHz, DMSO-d₆δ ppm 4.48 (s, 2H) 7.40 (s, 4H) 7.80 (s, 1H) 7.91 (s, 1H) 8.02 (s, 1H) 8.06 (s, 1H) 8.19 (s, 1H) 8.67 (s, 1H) 9.40-9.43 (m, 1H) 13.39 (s, 1H)

### Example 96

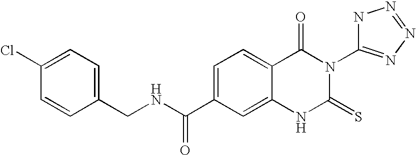

### N-(4-Chlorobenzyl-4-oxo-3-(1H-tetrazol-5-yl)-2-thioxo-1,2,3,4-tetrahydro-7-quinazolinecarboxamide

1H-Tetrazol-5-amine (281 mg, 3.3 mmol) was dissolved in DMSO (5 ml). Sodium hydride (95% by weight, 76 mg, 3 mmol) was added portionwise at room temperature with gas evolution. Methyl 4-[(4-chlorobenzylamino)carbonyl]-2-isothiocyanatobenzoate (82b, 361 mg, 1 mmol) was added to the reaction and the solution was stirred at rt overnight then poured into 1N HCl. Ethyl acetate was added and the mixture was shaken and filtered. The filtered solid was rinsed with ethyl acetate, 1N HCl, water, and hexanes and then air dried to give the title compound. Yield: 312 mg, 75%; LC/MS: M+1=414. ¹H-NMR (400 MHz, DMSO-d₆) δ ppm: 13.62 (s, 1H), 9.43 (t, J=6 Hz, 1H), 8.08 (d, J=8 Hz, 1H), 7.90 (d, J=1 Hz, 1H), 7.81 (dd, J=8, 1 Hz, 1H), 7.42 (d, J=8 Hz, 2H), 7.37 (d, J=8 Hz, 2H), 4.49 (d, J=6 Hz, 2H)

### Example 97

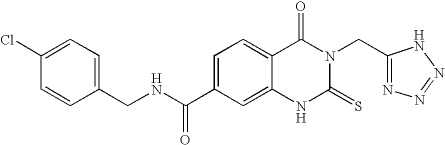

### N-(4-Chlorobenzyl)-4-oxo-3-(1H-tetrazol-5-ylmethyl)-2-thioxo-1,2,3,4-tetrahydro-7-quinazolinecarboxamide

A mixture of methyl 4-(4-chlorobenzylaminocarbonyl)-2-isothiocyanatobenzoate (82b, 280 mg) and 1 mmol (1H-tetrazol-5-ylmethyl)amine in DMF (5 ml) was heated at 70° C. for 1 hour and then added to a mixture of 1N hydrochloric acid and ethyl acetate. The precipitated solid was filtered, rinsed with water and ethyl acetate and dried to give N-(4-chlorobenzyl)-4-oxo-3-(1H-tetrazol-5-ylmethyl)-2-thioxo-1,2,3,4-tetrahydro-7-quinazolinecarboxamide. Yield: 95 mg, 29%; LC/MS: M+1=428; ¹H-NMR (400 MHz, DMSO-d₆) δ ppm: 13.38 (s, 1H), 9.40 (t, J=6 Hz, 1H), 8.09 (d, J=8 Hz, 1H), 7.89 (d, J=1 Hz, 1H), 7.80 (dd, J=8, 1 Hz, 1H), 7.42 (d, J=8 Hz, 2H), 7.37 (d, J=8 Hz, 2H), 5.91 (s, 2H), 4.48 (d, J=6 Hz, 2H)

### Example 98

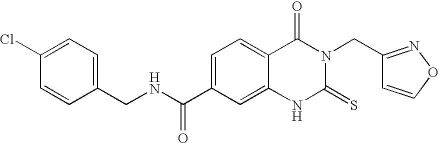

### N-(4-Chlorobenzyl)-3-(3-isoxazolylmethyl)-4-oxo-2-thioxo-1,2,3,4-tetrahydro-7-quinazolinecarboxamide

A mixture of methyl 4-(4-chlorobenzylaminocarbonyl)-2-isothiocyanatobenzoate (82b, 280 mg) and 1 mmol 3-(isoxazolylmethyl)amine was heated at 70° C. for 1.5 hours in DMF (5 ml) and triethylamine (0.15 ml) and then added to a mixture of 1N hydrochloric acid and ethyl acetate. The precipitated solid was filtered, rinsed with water and ethyl acetate and dried to give N-(4-chlorobenzyl)-3-(3-isoxazolylmethyl)-4-oxo-2-thioxo-1,2,3,4-tetrahydro-7-quinazolinecarboxamide. Yield: 100 mg, 30%; LC/MS: M+1=427; ¹H-NMR (400 MHz, DMSO-d₆) δ ppm: 13.28 (s, 1H), 9.38 (t, J=6 Hz, 1H), 8.84 (d, J=1 Hz, 1H), 8.06 (d, J=8 Hz, 1H), 7.87 (d, J=1 Hz, 1H), 7.78 (dd, J=8, 1 Hz, 1H), 7.41 (d, J=8 Hz, 2H), 7.36 (d, J=8 Hz, 2H), 6.59 (d, J=1 Hz, 1H), 5.72 (s, 2H), 4.48 (d, J=6 Hz, 2H) 8:1 isomers

### Example 99

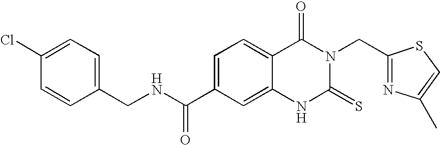

### N-(4-Chlorobenzyl)-3-[(4-methyl-1,3-thiazol-2-yl)methyl]-4-oxo-2-thioxo-1,2,3,4-tetrahydro-7-quinazolinecarboxamide

A mixture of methyl 4-(4-chlorobenzylaminocarbonyl)-2-isothiocyanatobenzoate (82b, 175 mg) and [(4-methyl-1,3-thiazol-2-yl)methyl]amine (175 mg) in DMSO (5 ml) was heated at 70° C. for 1 hour then added to 1N hydrochloric acid and the precipitate filtered off. The solid was rinsed with 1N hydrochloric acid and water and dried to give N-(4-chlorobenzyl)-3-[(4-methyl-1,3-thiazol-2-yl)methyl]-4-oxo-2-thioxo-1,2,3,4-tetrahydro-7-quinazolinecarboxamide. Yield: 220 mg, 99%; LC/MS: M+1=457; ¹H-NMR (400 MHz, DMSO-d₆) δ ppm: 13.30 (s, 1H), 9.38 (t, J=6 Hz, 1H), 8.07 (d, J=8 Hz, 1H), 7.86 (d, J=1 Hz, 1H), 7.78 (dd, J=8, 1 Hz, 1H), 7.41 (d, J=8 Hz, 2H), 7.36 (d, J=8 Hz, 2H), 7.16 (d, J=1 Hz, 1H), 5.89 (s, 2H), 4.48 (d, J=6 Hz, 2H), 2.30 (d, J=1 Hz, 3H)

### Example 100

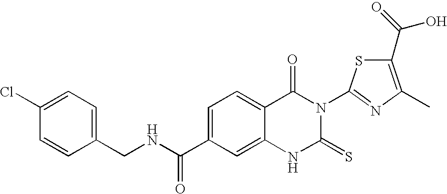

### 2-[7-(4-Chlorobenzylaminocarbonyl)-4-oxo-2-thioxo-1,4-dihydro-3(2H)-quinazolinyl]-4-methyl-1,3-thiazole-5-carboxylic acid

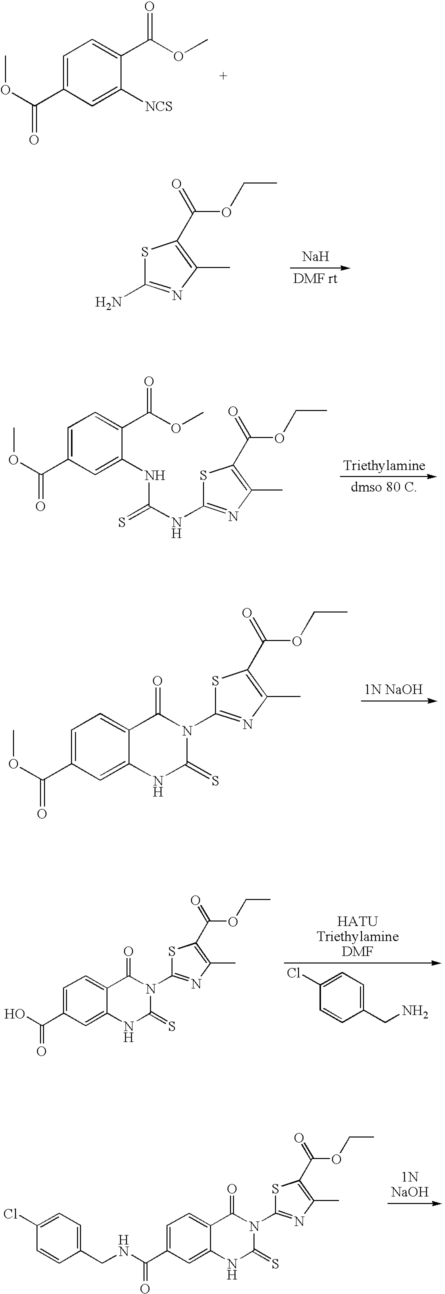

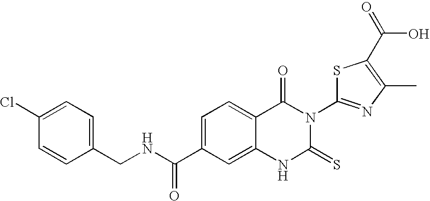

Dimethyl 2-isothiocyanato-1,4-benzenedicarboxylate (1a) and ethyl 2-amino-4-methyl-1,3-thiazole-5-carboxylate (2 mmol of each) were stirred at rt in DMSO (5 mL) and sodium hydride (2 mmol, 95%) was added slowly with gas evolution. The reaction was stirred for 1 hour and acidified with 1N hydrochloric acid. The solid was filtered off, rinsed with water and air dried. The solid was added to triethylamine (0.3 mL) and DMSO (5 mL) and heated at 80° C. for 2 hours. The reaction was acidified with 1N hydrochloric acid, filtered, rinsed with water and air dried. The solid was suspended in water (10 ml) and sodium hydroxide (2.8 ml) and ethanol (3 ml) were added and the reaction was stirred at rt overnight. LCMS showed 35% of the desired monoacid. The reaction was acidified with hydrochloric acid, filtered, rinsed with water and air dried. The solid was dissolved in DMF (10 ml) together with HATU (850 mg) and triethylamine (1 ml) then 4-chlorobenzylamine (300 mg) was added, the reaction was stirred at rt for one hour, acidified with 1N hydrochloric acid and extracted with ethyl acetate. The organic layer was washed with 1N, sat. sodium bicarbonate, and brine, dried over Na₂SO₄, and evaporated to give a solid (450 mg) that was 35% desired amide by LCMS. The solid was dissolved in methanol (3 ml) and added to water (10 ml). 1N Sodium hydroxide (2 ml) was added and the reaction was mixed at rt for 3 hours and then acidified with 1N hydrochloric acid, filtered, and air dried. The solid in DMSO was purified by HPLC to give 2-[7-(4-chlorobenzylaminocarbonyl)-4-oxo-2-thioxo-1,4-dihydro-3(2H)-quinazolinyl]-4-methyl-1,3-thiazole-5-carboxylic acid. Yield: 28 mg, 3%; LC/MS: M+1=487; ¹H-NMR (400 MHz, DMSO-d6) δ ppm: 13.46 (s, 1H), 9.42 (t, J=6 Hz, 1H), 8.06 (d, J=8 Hz, 1H), 7.87 (d, J=1 Hz, 1H), 7.79 (dd, J=8, 1 Hz, 1H), 7.42 (d, J=8 Hz, 2H), 7.37 (d, J=8 Hz, 2H), 4.49 (d, J=6 Hz, 2H), 2.65 (s, 3H)

### Example 101

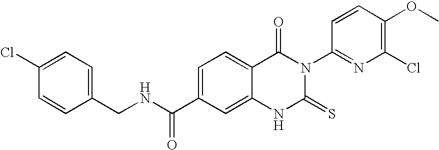

### 3-[6-Chloro-5-methoxy-2-pyridinyl]-N-(4-chlorobenzyl)-4-oxo-2-thioxo-1,2,3,4-tetrahydro-7-quinazolinecarboxamide

A rapidly stirred solution of 6-chloro-5-methoxy-2-pyridinamine (97 mg, 1.1 eq) and methyl 4-[(4-chlorobenzylamino)carbonyl]-2-isothiocyanatobenzoate (82b, 200 mg, 0.554 mmol, 1 eq.) in DMSO (2.5 ml) was heated to 65° C. under an inert atmosphere (N₂). LCMS confirmed consumption of the limiting reagent (82b) after 12 hours The reaction solution was filtered through a Teflon syringe filter and purified by reverse phase high pressure liquid chromatography (HPLC, acetonitrile/0.8% NaOH water, 15-75% gradient, 8 min, 50 ml/min) to produce the title compound as an off white solid. Yield: 78 mg, 29%, LC/MS: M+1=478, ¹H NMR (400 MHz, DMSO-d₆) δ ppm 3.98 (s, 3H), 4.49 (d, J=5.81 Hz, 2H), 7.32-7.47 (m, 4H), 7.55 (d, J=8.34 Hz, 1H), 7.75-7.85 (m, 2H), 7.89 (d, J=1.26 Hz, 1H), 8.06 (d, J=8.34 Hz, 1H), 9.41 (t, J=5.94 Hz, 1H), 13.32 (br. s., 1H)

### Example 102

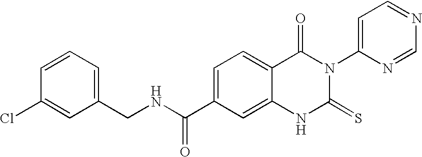

### N-(3-Chlorobenzyl)-4-oxo-3-(4-pyrimidinyl)-2-thioxo-1,2,3,4-tetrahydro-7-quinazolinecarboxamide

### 102a) Methyl 2-amino-4-(3-chlorobenzylaminocarbonyl)benzoate

To a stirred suspension of 3-amino-4-(methoxycarbonyl)benzoic acid (1.0 g, 5.13 mmol, 1 eq.) in DMF (7 mL) was added HATU (2.1 g, 0.5.64 mmol, 1.1 eq.) and DIEA (0.98 mL, 0.11 mol, 1.1 eq.) and the mixture was stirred at rt for 5 minutes until all dissolved. The resulting solution was added to 3-chlorobenzylamine (0.69 ml, 5.64 mmol, 1.1 eq.) in DMF (3 ml) via syringe and stirred at rt for 30 minutes when LCMS showed that the reaction was complete. The solution was added dropwise to ice-water with stirring and the precipitate collected by vacuum filtration, washed with ice-water and dried under vacuum to yield the title product which was used without further purification (94% purity by LCMS). Yield: 1.52 g, 93%; LC/MS: M+1=319; 1H NMR (400 MHz, DMSO-d₆) δ ppm 3.81 (s, 3H) 4.44 (d, J=6.06 Hz, 2H) 6.81 (s, 2H) 6.97 (d, J=8.59 Hz, 1H) 7.24-7.40 (m, 5H) 7.77 (d, J=8.59 Hz, 1H) 9.07 (t, J=6.06 Hz, 1H)

### 102b) Methyl 4-(3-chlorobenzylaminocarbonyl)-2-isothiocyanatobenzoate

To a rapidly stirred solution of methyl 2-amino-4-(3-chlorobenzylaminocarbonyl)benzoate (102a, 318 mg, 1.0 mmol, 1 eq) in sat sodium bicarbonate (5 mL) and chloroform (5 mL), thiophosgene (0.092 ml, 1.2 mmol, 1.2 eq) was added dropwise. After stirring at rt for 30 minutes, the product was extracted with DCM and the combined organic layers were dried over anhydrous sodium sulfate and concentrated then dried in vacuo. The resulting light tan solid was used without further purification. Yield: 362 mg, 98%, 97% purity; LC/MS: M+1=361; 1H NMR (400 MHz, DMSO-d₆) δ ppm 3.91 (s, 3H) 4.49 (d, J=6.06 Hz, 2H) 7.26-7.43 (m, 4H) 7.94 (dd, J=8.08, 1.77 Hz, 1H) 8.02-8.10 (m, 2H) 9.35 (t, J=6.06 Hz, 1H)

### 102c) N-(3-Chlorobenzyl)-4-oxo-3-(4-pyrimidinyl)-2-thioxo-1,2,3,4-tetrahydro-7-quinazolinecarboxamide

A mixture of 4-(3-chlorobenzylaminocarbonyl)-2-isothiocyanatobenzoate (102b, 100 mg, 0.278 mmol, 1 eq.) and 4-pyrimidinamine (26.4 mg, 0.278 mmol, 1.0 eq.) in DMSO (2 mL) was heated overnight at 80° C. with stirring. DIEA (1 mL) was added to the reaction mixture and stirred at rt for 10 minutes. The mixture was filtered and purified by HPLC (10-90% gradient system in 8 minutes) under acidic condition to give the title product. Yield: 15.4 mg, 13%; LCMS M+H=424; ¹H NMR (400 MHz, DMSO-d₆) δ ppm 4.51 (d, J=5.81 Hz, 2H) 7.28-7.45 (m, 4H) 7.78 (dd, J=5.05, 1.26 Hz, 1H) 7.81 (dd, J=8.34, 1.52 Hz, 1H) 7.91 (d, J=1.52 Hz, 1H) 8.08 (d, J=8.34 Hz, 1H) 9.06 (d, J=5.05 Hz, 1H) 9.33 (s, 1H) 9.43 (t, J=5.94 Hz, 1H) 13.44 (s, 1H)

### Example 103

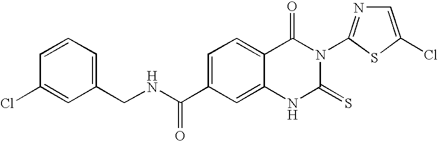

### N-(3-Chlorobenzyl)-3-(5-chloro-1,3-thiazol-2-yl)-4-oxo-2-thioxo-1,2,3,4-tetrahydro-7-quinazolinecarboxamide

To a rapidly stirred solution of 5-chloro-1,3-thiazol-2-amine (142 mg, 1.5 eq) in DMSO (3.5 ml) was added sodium hydride (NaH, 15.9 mg, 1.2 eq) and the vessel was quickly capped to minimize exposure to air. After stirring at rt for 10 minutes, methyl 4-[(3-chlorobenzylamino)carbonyl]-2-isothiocyanatobenzoate (102b, 200 mg, 0.554 mmol, 1 eq.) was added as a solution in DMSO (0.369M) via a syringe. The reaction mixture was stirred rapidly at rt overnight. LCMS indicated the reaction had not progressed so the mixture was heated to 60° C. with stirring. Reaction progress was monitored by LCMS and after consumption of the limiting reagent (102b, 48 hours) the reaction solution was filtered through a Teflon syringe filter and purified by reverse phase high pressure liquid chromatography (HPLC, 25-70% gradient, 8 min, 50 ml/min) to produce the title compound as an off white solid. Yield: 24 mg, 9.2%, LC/MS: M+1=463, ¹H NMR (400 MHz, DMSO-d₆) δ ppm 4.50 (d, J=5.81 Hz, 2H), 7.27-7.44 (m, 4H), 7.79 (dd, J=8.21, 1.39 Hz, 1H), 7.87 (d, J=1.26 Hz, 1H), 7.93 (s, 1H), 8.06 (d, J=8.34 Hz, 1H), 9.42 (t, J=6.06 Hz, 1H), 13.44 (s, 1H)

### Example 104

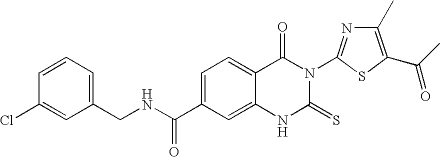

### 3-(5-Acetyl-4-methyl-1,3-thiazol-2-yl)-N-(3-chlorobenzyl)-4-oxo-2-thioxo-1,2,3,4-tetrahydro-7-quinazolinecarboxamide

To a rapidly stirred solution of 1-(2-amino-4-methyl-1,3-thiazol-5-yl)ethanone (130 mg, 1.5 eq) in DMSO (3.5 ml) was added sodium hydride (NaH, 15.9 mg, 1.2 eq), the vessel was quickly capped to minimize exposure to air and stirred at rt for 10 minutes. Methyl 4-[(3-chlorobenzylamino)carbonyl]-2-isothiocyanatobenzoate (102b, 200 mg, 0.554 mmol, 1 eq.) was added as a solution in DMSO (0.369M) via a syringe. The reaction mixture was stirred rapidly at rt overnight. LCMS indicated the reaction had not progressed so the mixture was heated to 60° C. with stirring for 48 hours after which LCMS indicated no change in reaction progress. The temperature was increased to 80° C. with stirring and after an additional 24 hours, consumption of the limiting reagent (102b) was confirmed by LCMS. The reaction solution was filtered through a Teflon syringe filter and purified by reverse phase high pressure liquid chromatography (HPLC, 35-60% gradient, 8 min, 50 ml/min) to produce the title compound as an off white solid. Yield: 67 mg, 25%, LC/MS: M+1=485, ¹H NMR (400 MHz, MeOD-d₄) δ ppm 2.60 (s, 3H), 2.68 (s, 3H), 4.50 (d, J=6.06 Hz, 2H), 7.28-7.43 (m, 4H), 7.79 (dd, J=8.21, 1.39 Hz, 1H), 7.87 (d, J=1.01 Hz, 1H), 8.06 (d, J=8.34 Hz, 1H), 9.43 (t, J=5.94 Hz, 1H), 13.46 (s, 1H)

### Example 105

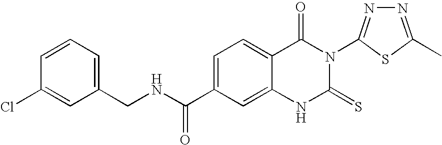

### N-[(3-chlorophenyl)methyl]-3-(5-methyl-1,3,4-thiadiazol-2-yl)-4-oxo-2-thioxo-1,2,3,4-tetrahydro-7-quinazolinecarboxamide

To a rapidly stirred solution of 5-methyl-1,3,4-thiadiazol-2-amine (96 mg, 1.5 eq) in DMSO (3.5 ml) was added sodium hydride (NaH, 15.9 mg, 1.2 eq), the vessel was quickly capped to minimize exposure to air and stirred at rt for 10 minutes. Methyl 4-[(3-chlorobenzylamino)carbonyl]-2-isothiocyanatobenzoate (102b, 200 mg, 0.554 mmol, 1 eq.) was added as a solution in DMSO (0.369M) via a syringe. The reaction mixture was rapidly stirred at rt overnight. LCMS indicated the reaction had not progressed so the reaction mixture was heated to 60° C. with stirring. LCMS confirmed consumption of the limiting reagent (1b) after 48 hours. The reaction solution was filtered through a Teflon syringe filter and purified by reverse phase high pressure liquid chromatography (HPLC, 35-60% gradient, 8 min, 50 ml/min) to produce the title compound as an off white solid. Yield: 72 mg, 29%, LC/MS: M+1=443, ¹H NMR (400 MHz, DMSO-d₆) δ ppm 2.81 (s, 3H), 4.51 (d, J=6.06 Hz, 2H), 7.24-7.49 (m, 4H), 7.80 (dd, J=8.34, 1.52 Hz, 1H), 7.88 (d, J=1.26 Hz, 1H), 8.07 (d, J=8.08 Hz, 1H), 9.43 (t, J=5.94 Hz, 1H), 13.50 (s, 1H)

### Example 106

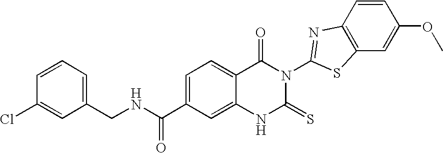

### N-[(3-chlorophenyl)methyl]-3-[6-(methyloxy)-1,3-benzothiazol-2-yl]-4-oxo-2-thioxo-1,2,3,4-tetrahydro-7-quinazolinecarboxamide

To a rapidly stirred solution of 6-methoxy-1,3-benzothiazol-2-amine (150 mg, 1.5 eq) in DMSO (3.5 ml) was added sodium hydride (NaH, 15.9 mg, 1.2 eq). The vessel was quickly capped to minimize exposure to air and stirred at rt for 10 minutes. Methyl 4-[(3-chlorobenzylamino)carbonyl]-2-isothiocyanatobenzoate (102b, 200 mg, 0.554 mmol, 1 eq.) was added as a solution in DMSO (0.369M) via a syringe. The reaction mixture was rapidly stirred at rt overnight. LCMS indicated the reaction had not progressed so the reaction mixture was heated to 60° C. with stirring, After 48 hours LCMS confirmed consumption of the limiting reagent (102b). The reaction solution was filtered through a Teflon syringe filter and purified by reverse phase high pressure liquid chromatography (HPLC, 30-70% gradient, 8 min, 50 ml/min) to produce the title compound as an off white solid. Yield: 55 mg, 19.3%, LC/MS: M+1=509, ¹H NMR 1H NMR (400 MHz, DMSO-d₆) δ ppm 3.87 (s, 3H), 4.51 (d, J=5.81 Hz, 2H), 7.17 (dd, J=8.84, 2.53 Hz, 1H), 7.28-7.46 (m, 4H), 7.76 (d, J=2.78 Hz, 1H), 7.80 (dd, J=8.34, 1.26 Hz, 1H), 7.89 (d, J=1.01 Hz, 1H), 7.94 (d, J=9.09 Hz, 1H), 8.08 (d, J=8.08 Hz, 1H), 9.44 (t, J=6.06 Hz, 1H), 13.47 (s, 1H)

### Example 107

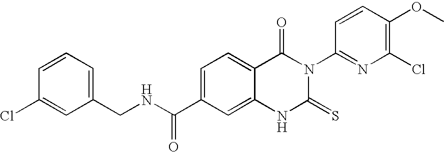

### 3-[6-Chloro-5-methoxy-2-pyridinyl]-N-(3-chlorobenzyl)-4-oxo-2-thioxo-1,2,3,4-tetrahydro-7-quinazolinecarboxamide

A rapidly stirred solution of 6-chloro-5-methoxy-2-pyridinamine (150 mg, 1.3 eq) and methyl 4-[(3-chlorobenzylamino)carbonyl]-2-isothiocyanatobenzoate (102b, 200 mg, 0.554 mmol, 1 eq.) in DMSO (2 ml) was heated to 80° C. LCMS confirmed consumption of the limiting reagent (102b) after 12 hours. The reaction solution was filtered through a Teflon syringe filter and purified by reverse phase high pressure liquid chromatography (HPLC, 30-70% gradient, 8 min, 50 ml/min) to produce the title compound as an off white solid. Yield: 68 mg, 25%, LC/MS: M+1=487, ¹H NMR (400 MHz, DMSO-d₆) δ ppm 3.99 (s, 3H), 4.51 (d, J=5.81 Hz, 2H), 7.26-7.47 (m, 4H), 7.55 (d, J=8.59 Hz, 1H), 7.79 (dd, J=8.21, 1.39 Hz, 2H), 7.90 (d, J=1.01 Hz, 1H), 8.07 (d, J=8.34 Hz, 1H), 9.43 (t, J=5.94 Hz, 1H), 13.33 (s, 1H)

### Example 108

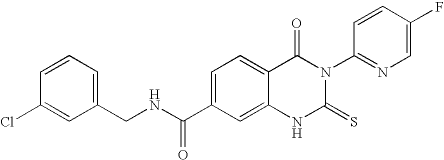

### N-(3-Chlorobenzyl)-3-(5-fluoro-2-pyridinyl)-4-oxo-2-thioxo-1,2,3,4-tetrahydro-7-quinazolinecarboxamide

A rapidly stirred solution of 5-fluoro-2-pyridinamine (81 mg, 1.3 eq) and methyl 4-[(3-chlorobenzylamino)carbonyl]-2-isothiocyanatobenzoate (102b, 200 mg, 0.554 mmol, 1 eq.) in DMSO (2 ml) was heated to 80° C. LCMS confirmed consumption of the limiting reagent (102b) after 12 hours. The reaction solution was filtered through a Teflon syringe filter and purified by reverse phase high pressure liquid chromatography (HPLC, 30-70% gradient, 8 min, 50 ml/min) to produce the title compound as an off white solid. Yield: 55 mg, 22%, LC/MS: M+1=441, ¹H NMR (400 MHz, DMSO-d₆) δ ppm 4.51 (d, J=5.81 Hz, 2H), 7.29-7.46 (m, 4H), 7.64 (dd, J=8.72, 4.17 Hz, 1H), 7.80 (dd, J=8.34, 1.52 Hz, 1H), 7.91 (d, J=1.26 Hz, 1H), 7.97 (td, J=8.46, 3.03 Hz, 1H), 8.07 (d, J=8.08 Hz, 1H), 8.62 (d, J=3.03 Hz, 1H), 9.42 (t, J=5.94 Hz, 1H), 13.33 (s, 1H)

### Example 109

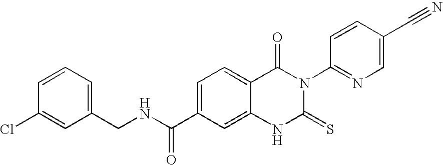

### N-(3-Chloropbnyzl)-3-(5-cyano-2-pyridinyl)-4-oxo-2-thioxo-1,2,3,4-tetrahydro-7-quinazolinecarboxamide

A rapidly stirred solution of 6-amino-3-pyridinecarbonitrile (86 mg, 1.3 eq) and methyl 4-[(3-chlorobenzylamino)carbonyl]-2-isothiocyanatobenzoate (102b, 200 mg, 0.554 mmol, 1 eq.) in DMSO (2 ml) was heated to 80° C. LCMS confirmed consumption of the limiting reagent (102b) after 12 hours. The reaction solution was filtered through a Teflon syringe filter and purified by reverse phase high pressure liquid chromatography (HPLC, 30-70% gradient, 8 min, 50 ml/min) to produce the title compound as an off white solid. Yield: 27 mg, 11%, LC/MS: M+1=448, ¹H NMR (400 MHz, DMSO-d₆) δ ppm 4.51 (d, J=6.06 Hz, 2H), 7.28-7.46 (m, 4H), 7.74-7.87 (m, 2H), 7.91 (d, J=1.01 Hz, 1H), 8.08 (d, J=8.08 Hz, 1H), 8.56 (dd, J=8.21, 2.15 Hz, 1H), 9.13 (dd, J=2.27, 0.76 Hz, 1H), 9.43 (t, J=5.94 Hz, 1H), 13.43 (s, 1H)

### Example 110

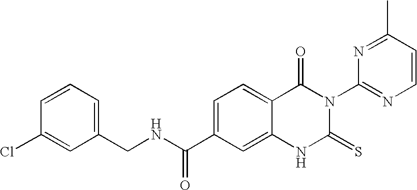

### N-(3-Chlorobenzyl)-3-(4-methyl-2-pyrimidinyl)-4-oxo-2-thioxo-1,2,3,4-tetrahydro-7-quinazolinecarboxamide

A rapidly stirred solution of 4-methyl-2-pyrimidinamine (86 mg, 1.3 eq) and methyl 4-[(3-chlorobenzylamino)carbonyl]-2-isothiocyanatobenzoate (102b, 200 mg, 0.554 mmol, 1 eq.) in DMSO (2 ml) was heated to 80° C. LCMS confirmed consumption of the limiting reagent (102b) after 12 hours. The reaction solution was filtered through a Teflon syringe filter and purified by reverse phase high pressure liquid chromatography (HPLC, 20-60% gradient, 8 min, 50 ml/min) to produce the title compound as an off white solid. Yield: 27 mg, 11%, LC/MS: M+1=438, ¹H NMR (400 MHz, DMSO-d₆) δ ppm 2.55 (s, 3H), 4.51 (d, J=6.06 Hz, 2H), 7.27-7.46 (m, 4H), 7.54 (d, J=5.05 Hz, 1H), 7.81 (dd, J=8.34, 1.26 Hz, 1H), 7.92 (d, J=1.26 Hz, 1H), 8.09 (d, J=8.34 Hz, 1H), 8.86 (d, J=5.05 Hz, 1H), 9.43 (t, J=5.94 Hz, 1H), 13.42 (s, 1H)

### Example 111

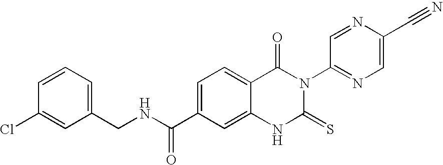

### N-(3-Chlorobenzyl)-3-(5-cyano-2-pyrazinyl)-4-oxo-2-thioxo-1,2,3,4-tetrahydro-7-quinazolinecarboxamide

To a rapidly stirred solution of 5-amino-2-pyrazinecarbonitrile (87 mg, 1.3 eq) under an inert atmosphere (N₂) and in DMSO (2 ml) was added sodium hydride (NaH, 13.3 mg, 1 eq) and stirred at rt for 10 minutes. A solution of methyl 4-[(3-chlorobenzylamino)carbonyl]-2-isothiocyanatobenzoate (102b, 200 mg, 0.554 mmol, 1 eq.) in DMSO (1 ml) was added via a syringe and the mixture stirred at rt. LCMS confirmed consumption of the limiting reagent (102b) after 12 hours. The reaction solution was filtered through a Teflon syringe filter and purified by reverse phase high pressure liquid chromatography utilizing basic eluent (HPLC, Phenomenex Gemini 10u C18 110A, 50×100 mm 10 micron column, acetonitrile/0.1% NH4OH/water eluant, 7-47% gradient, 23 min, 147 ml/min) to produce the title compound as an yellow solid. Yield: 27 mg, 11%, LC/MS: M+1=449, ¹H NMR (600 MHz, DMSO-d₆) δ ppm 4.51 (d, J=6.18 Hz, 2H), 7.33 (t, J=6.89 Hz, 2H), 7.36-7.44 (m, 2H), 7.65 (d, J=6.65 Hz, 1H), 7.83 (s, 1H), 7.99 (d, J=8.08 Hz, 1H), 8.94 (br. s., 1H), 9.29 (s, 1H), 9.35 (br. s., 1H)

### Example 112

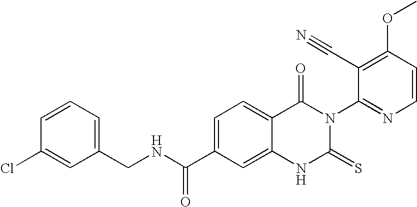

### N-(3-Chlorobenzyl)-3-[3-cyano-4-methoxy-2-pyridinyl]-4-oxo-2-thioxo-1,2,3,4-tetrahydro-7-quinazolinecarboxamide

A rapidly stirred solution of 2-amino-4-methoxy-3-pyridinecarbonitrile (91 mg, 1.1 eq) and methyl 4-[(3-chlorobenzylamino)carbonyl]-2-isothiocyanatobenzoate (102b, 200 mg, 0.554 mmol, 1 eq.) in DMSO (2 ml) was heated to 65° C. LCMS confirmed consumption of the limiting reagent (102b) after 12 hours. The reaction solution was filtered through a Teflon syringe filter and purified by reverse phase high pressure liquid chromatography (HPLC, Phenomenex Gemini 10u C18 110A, 50×100 mm 10 micron column, acetonitrile/0.1% NH4OH/water eluant, 7-47% gradient, 23 min, 147 ml/min) to produce the title compound as an off white solid. Yield: 25 mg, 9.3%, LC/MS: M+1=478, ¹H NMR (400 MHz, DMSO-d₆) δ ppm 4.12 (s, 3H), 4.51 (d, J=5.81 Hz, 2H), 7.36 (dd, J=17.56, 7.96 Hz, 4H), 7.51 (d, J=6.32 Hz, 1H), 7.84 (d, J=7.83 Hz, 1H), 7.93 (s, 1H), 8.12 (d, J=8.08 Hz, 1H), 8.79 (d, J=6.06 Hz, 1H), 9.45 (t, J=5.81 Hz, 1H), 13.65 (s, 1H)

### Example 113

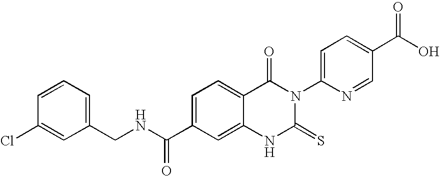

### 6-[7-(3-Chlorobenzylaminocarbonyl)-4-oxo-2-thioxo-1,4-dihydro-3(2H)-quinazolinyl]-3-pyridinecarboxylic acid

### 113a) DMHB resin-bound 3-chlorobenzylamine

To a mixture of DMHB resin (10 g, 1.5 mmol/g loading, 15 mmol) in NMP1 (20 mL), was added 3-chlorobenzylamine (10.6 g, 75 mmol), HOAc (10 mL), and sodium triacetoxyborohydride (19 g, 90 mmol). The mixture was shaken at rt overnight (releasing the pressure for a few times at the beginning of reaction to avoid pressure build-up) and was then washed with NMP (100 mL×3), DCM (100 mL×3), methanol (100 mL×3) and DCM (100 mL×3). The resulting resin was dried under vacuum overnight to yield DMHB resin bound 3-chlorobenzylamine (12.7 g).

### 113b) DMHB resin bound methyl 2-amino-4-(3-chlorobenzylaminocarbonyl)benzoate

To 3-amino-4-(methoxycarbonyl)benzoic acid (2.65 g, 13.6 mmol) in DMF (60 mL) was added HATU (5.17 g, 13.6 mmol) and then DIEA (2.4 mL, 13.6 mmol). The mixture was stirred at RT for 10 min and then added to a 125-mL-shaker containing the above DMHB resin bound 3-chlorobenzylamine (113a, 3.0 g, 1.26 mmol/g (theoretical loading), 3.78 mmol). The mixture was shaken at rt overnight and was then washed successively with DMF (20 mL×2), water (20 mL×2), DMF (20 mL×2), DCM (20 mL×2), methanol (20 mL×2), DCM (20 mL×2) and methanol (20 mL×2). The resulting resin was dried under vacuum for overnight to yield DMHB resin bound methyl 2-amino-4-(3-chlorobenzylaminocarbonyl)benzoate (3.85 g). An analytical amount of the resin was cleaved with 40% of TFA in DCE for 10 min. The resulting solution was concentrated in vacuo and dissolved in 0.5 mL of methanol for LCMS analysis. LCMS showed 100% purity; MS (ESI): 318.5 [M+H]⁺.

### 113c) DMHB resin bound methyl 4-(3-chlorobenzylaminocarbonyl)-2-isothiocyanatobenzoate

DMHB resin bound methyl 2-amino-4-({[(3-chlorophenyl)methyl]amino}carbonyl)benzoate (113b, 3 g, (theoretical loading 1.03 mmol/g), 3.09 mmol) in chloroform (40 mL) and sat aqueous sodium bicarbonate solution (40 ml) was shaken at RT for 10 minutes. Thiophosgene (1.2 mL, 15.45 mmol) was added slowly then the mixture was shaken at rt overnight. The resin was washed with chloroform (20 mL×2), water (20 mL×2), methanol (20 mL×2), DCM (20 mL×2), methanol (20 mL×2) and DCM (20 mL×2). The resulting resin was dried under vacuum for overnight to yield DMHB resin bound methyl 4-(3-chlorobenzylaminocarbonyl)-2-isothiocyanatobenzoate (3.0 g). An analytical amount of the resin was cleaved with 40% of TFA in DCE for 10 minutes. The resulting solution was concentrated in vacuo and dissolved in 0.5 mL of methanol for LCMS analysis. LCMS showed 93% purity; MS (ESI): 361 [M+H]⁺.

### 113d) 6-[7-(3-Chlorobenzylaminocarbonyl)-4-oxo-2-thioxo-1,4-dihydro-3(2H)-quinazolinyl]-3-pyridinecarboxylic acid

To DMHB resin bound methyl 4-(3-chlorobenzylaminocarbonyl)-2-isothiocyanatobenzoate (113c, 300 mg, (theoretical loading 0.99 mmol/g), 0.297 mmol) in DMSO (4 ml) in a vial was added 6-amino-3-pyridinecarboxylic acid (123 mg, 0.891 mmol) and the mixture was shaken at 60° C. overnight. 2M sodium hydroxide (0.05 ml) was added and the resulting mixture was shaken at 60° C. overnight. The resin was washed twice each with DMSO, water, methanol, DCM, and methanol and dried under vacuum to give DMHB resin bound 6-[7-(3-chlorobenzylaminocarbonyl)-4-oxo-2-thioxo-1,4-dihydro-3(2H)-quinazolinyl]-3-pyridinecarboxylic acid.

To the above resin bound 6-[7-(3-chlorobenzylaminocarbonyl)-4-oxo-2-thioxo-1,4-dihydro-3(2H)-quinazolinyl]-3-pyridinecarboxylic acid was added 40% TFA in DCM (4 mL) and shaken at rt for 30 min. The resin was filtered and rinsed with DCE and the filtrate was concentrated to dryness. The residue was dissolved in DMSO and purified by HPLC under acidic condition to yield the title compound. Yield: 12.7 mg, 9.2%; MS, M+H+=467; 1H NMR (400 MHz, DMSO-d₆) δ ppm 4.51 (d, J=5.56 Hz, 2H) 7.28-7.44 (m, 4H) 7.70 (d, J=8.84 Hz, 1H) 7.80 (dd, J=8.21, 1.64 Hz, 1H) 7.91 (d, J=1.52 Hz, 1H) 8.08 (d, J=8.08 Hz, 1H) 8.46 (dd, J=8.08, 2.27 Hz, 1H) 9.08 (d, J=2.27 Hz, 1H) 9.42 (t, J=6.19 Hz, 1H) 13.37 (s, 1H) 13.64 (br. s., 1H)

### Biological Background:

The following references set out information about the target enzymes, HIF prolyl hydroxylases, and methods and materials for measuring inhibition of same by small molecules.
M. Hirsilä, P. Koivunen, V. Günzler, K. I. Kivirikko, and J. Myllyharju “Characterization of the Human Prolyl 4-Hydroxylases That Modify the Hypoxia-inducible Factor” J. Biol. Chem., 2003, 278, 30772-30780.
C. Willam, L. G. Nicholls, P. J. Ratcliffe, C. W. Pugh, P. H. Maxwell “The prolyl hydroxylase enzymes that act as oxygen sensors regulating destruction of hypoxia-inducible factor α” Advan. Enzyme Regul., 2004, 44, 75-92
M. S. Wiesener, J. S. Jürgensen, C. Rosenberger, C. K. Scholze, J. H. Hörstrup, C. Warnecke, S. Mandriota, I. Bechmann, U. A. Frei, C. W. Pugh, P. J. Ratcliffe, S. Bachmann, P. H. Maxwell, and K.-U. Eckardt “Widespread hypoxia-inducible expression of HIF-2α in distinct cell populations of different organs” FASEB J., 2003, 17, 271-273.
S. J. Klaus, C. J. Molineaux, T. B. Neff, V. Guenzler-Pukall, I. Lansetmo Parobok, T. W. Seeley, R. C. Stephenson “Use of hypoxia-inducible factor α (HIFα) stabilizers for enhancing erythropoiesis” PCT Int. Appl. (2004), WO 2004108121 A1
C. Warnecke, Z. Zaborowska, J. Kurreck, V. A. Erdmann, U. Frei, M. Wiesener, and K.-U. Eckardt “Differentiating the functional role of hypoxia-inducible factor (HIF)-1α and HIF-2α (EPAS-1) by the use of RNA interference: erythropoietin is a HIF-2α target gene in Hep3B and Kelly cells” FASEB J., 2004, 18, 1462-1464.
   For the expression of EGLN3 see:
R. K. Bruick and S. L. McKnight “A Conserved Family of Prolyl-4-Hydroxylases That Modify HIF” Science, 2001, 294, 1337-1340.
   For the expression of HIF2α-CODD see:
a) P. Jaakkola, D. R. Mole, Y.-M. Tian, M. I. Wilson, J. Gielbert, S. J. Gaskell, A. von Kriegsheim, H. F. Hebestreit, M. Mukherji, C. J. Schofield, P. H. Maxwell, C. W. Pugh, P, J. Ratcliffe “Targeting of HIF-α to the von Hippel-Lindau Ubiquitylation Complex by O₂-Regulated Prolyl Hydroxylation” Science, 2001, 292, 468-472.
b) M. Ivan, K. Kondo, H. Yang, W. Kim, J. Valiando, M. Ohh, A. Salic, J. M. Asara, W. S. Lane, W. G. Kaelin Jr. “HIFα Targeted for VHL-Mediated Destruction by Proline Hydroxylation: Implications for O₂Sensing” Science, 2001, 292, 464-468.
   For the expression of VHL, elongin b and elongin c see:
A. Pause, S. Lee, R. A. Worrell, D. Y. T. Chen, W. H. Burgess, W. M. Linehan, R. D. Klausner “The von Hippel-Lindau tumor-suppressor gene product forms a stable complex with human CUL-2, a member of the Cdc53 family of proteins” Proc. Natl. Acad. Sci. USA, 1997, 94, 2156-2161.

### Biological Assay(s)

### EGLN3 Assay

### Materials:

His-MBP-EGLN3 (6HisMBPAttB1EGLN3(1-239)) was expressed in E. Coli and purified from an amylase affinity column. Biotin-VBC [6H is SumoCysVHL(2-213), 6HisSumoElonginB(1-118), and 6His SumoElonginC(1-112)] and His-GB1-HIF2α-CODD (6HisGB1tevHIF2A(467-572)) were expressed from E. Coli.

### Method:

Cy5-labelled HIF2α CODD, and a biotin-labeled VBC complex were used to determine EGLN3 inhibition. EGLN3 hydroxylation of the Cy5CODD substrate results in its recognition by the biotin-VBC. Addition of a Europium/streptavidin (Eu/SA) chelate results in proximity of Eu to Cy5 in the product, allowing for detection by energy transfer. A ratio of Cy5 to Eu emission (LANCE Ratio) is the ultimate readout, as this normalized parameter has significantly less variance than the Cy5 emission alone.

Then 50 nL of inhibitors in DMSO (or DMSO controls) were stamped into a 384-well low volume Corning NBS plate, followed by addition of 2.5μL of enzyme [50 mL buffer (50 mM HEPES/50 mM KCl)+1 mL of a 10 mg/mL BSA in buffer+6.25μL of a 10 mg/mL FeCl₂solution in water+100μL of a 200 mM solution of ascorbic acid in water+15.63μL EGLN3] or control [50 mL buffer+1 mL of a 10 mg/mL BSA in buffer+6.25μL of a 10 mg/mL FeCl₂solution in water+100μL of a 200 mM solution of ascorbic acid in water]. Following a 3 minutes incubation, 2.5μL of substrate [50 mL Buffer+68.6μL biotin-VBC+70.4μL Eu (at 710 μg/mL stock)+91.6μL Cy5CODD+50μL of a 20 mM solution of 2-oxoglutaric acid in water+0.3 mM CHAPS] was added and incubated for 30 minutes. The plate was loaded into a PerkinElmer Viewlux for imaging. For dose response experiments, normalized data were fit by ABASE/XC50 using the equation y=a+(b−a)/(1+(10̂x/10̂c)̂d), where a is the minimum % activity, b is the maximum % activity, c is the pIC₅₀, and d is the Hill slope.

The IC₅₀for exemplified compounds in the EGLN3 assay ranged from approximately 1-100 nanomolar. This range represents the data accumulated as of the time of the filing of this initial application. Later testing may show variations in IC₅₀data due to variations in reagents, conditions and variations in the method(s) used from those given herein above. So this range is to be viewed as illustrative, and not a absolute set of numbers.

### Measure Epo Protein Produced by Hep3B Cell Line Using ELISA Method.

Hep3B cells obtained from the American Type Culture Collection (ATCC) are seeded at 2×10̂4 cells/well in Dulbecco's Modified Eagle Medium (DMEM)+10% FBS in 96-well plates. Cells are incubated at 37 deg C/5% CO2/90% humidity (standard cell culture incubation conditions). After overnight adherence, medium is removed and replaced with DMEM without serum containing test compound or DMSO negative control. Following 48 hours incubation, cell culture medium is collected and assayed by ELISA to quantitate Epo protein.

The EC₅₀for exemplar compounds in the Hep3B ELISA assay ranged from approximately 1-20 micromolar using the reagents and under the conditions outlined herein above. This range represents the data accumulated as of the time of the filing of this initial application. Later testing may show variations in EC₅₀data due to variations in reagents, conditions and variations in the method(s) used from those given herein above. So this range is to be viewed as illustrative, and not a absolute set of numbers.

These compound are believed to be useful in therapy as defined above and to not have unacceptable or untoward effects when used in compliance with a permitted therapeutic regime.

The foregoing examples and assay have been set forth to illustrate the invention, not limit it. What is reserved to the inventors is to be determined by reference to the claims.

1. A compound of formula (I): 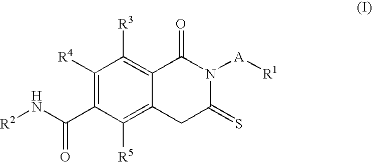
wherein:
R¹is an unsubstituted or substituted 4 to 8-membered mono-cyclic heteroaryl, a 9-11-membered bicyclic heteroaryl ring or an unsubstituted or substituted 4 to 8-membered heterocycloalkyl ring; each containing one or more hetero atoms selected from the group consisting of N, O and S; wherein a carbon atom in any of said rings can be substituted by one or more groups selected from the group consisting of C₁-C₆alkyl, halogen, —OR⁶, —CN, —C(O)R⁶, and —C(O)OR⁶;
A is a bond, or CR⁷R⁸, or NR⁶;
R²is aryl, C₁-C₁₀alkyl-aryl, heteroaryl, alkyl-heteroaryl, C₃-C₈cycloalkyl-heterocyclyl, C₁-C₁₀alkyl, C₂-C₁₀alkenyl, C₂-C₁₀alkynyl, C₃-C₈cycloalkyl, C₃-C₈heterocycloalkyl, C₅-C₈cycloalkenyl, C₂-C₁₀alkyl-R⁹;
R³, R⁴and R⁵each independently selected from the group consisting of hydrogen, nitro, cyano, halogen, CF₃, —C(O)R⁶, —C(O)OR⁶, —OR⁶, —SR⁶, —S(O)R⁶, —S(O)₂R⁶, —NR⁷R⁸, —CONR⁷R⁸, —N(R⁷)C(O)R⁶, —N(R⁷)C(O)OR⁶, —OC(O)NR⁷R⁸, —N(R⁷)C(O)N⁷R⁸, —P(O)(OR⁶)₂, —SO₂NR⁷R⁸, —N(R⁷)SO₂R⁶, C₁-C₁₀alkyl, C₂-C₁₀alkenyl, C₂-C₁₀alkynyl, C₃-C₈cycloalkyl, C₃-C₈heterocycloalkyl, C₅-C₈cycloalkenyl, aryl, and heteroaryl;
each R⁶is independently selected from the group consisting of hydrogen, C₁-C₁₀alkyl, C₃-C₁₀cycloalkyl, C₃-C₁₀heterocycloalkyl, aryl, and heteroaryl;
R⁷and R⁸are each independently selected from the group consisting of hydrogen, C₁-C₁₀alkyl, C₃-C₁₀cycloalkyl, C₃-C₁₀heterocycloalkyl, aryl, and heteroaryl; or R⁷and R⁸taken together with the nitrogen to which they are attached form a 5- or 6- or 7-membered saturated ring optionally containing one other heteroatom which is oxygen, nitrogen or sulphur;
R⁹is selected from the group consisting of nitro, cyano, halogen, —C(O)R⁶, —C(O)OR⁶, —OR⁶, —SR⁶, —S(O)R⁶, —S(O)₂R⁶, —NR⁷R⁸, —CONR⁷R⁸, —N(R⁷)C(O)R⁶, —N(R⁷)C(O)OR⁶, —OC(O)NR⁷R⁸, —N(R⁷)C(O)N⁷R⁸, —P(O)(OR⁶)₂, —SO₂NR⁷R⁸, —N(R⁷)SO₂R⁸; and
any carbon or heteroatom of R², R³, R⁴, R⁵, R⁶, R⁷, R⁸, R⁹, where possible, may be substituted with one or more substituents independently selected from the group consisting of C₁-C₆alkyl, aryl, heteroaryl, halogen, —OR⁶, —NR⁷R⁸, cyano, nitro, —C(O)R⁶, —C(O)OR⁶, —SR⁶, —S(O)R⁶, —S(O)₂R⁶, —CONR⁷R⁸, —N(R⁷)C(O)R⁶, —N(R⁷)C(O)OR⁶, —OC(O)NR⁷R⁸, —N(R⁷)C(O)NR⁷R⁸, —SO₂NR⁷R⁸, —N(R⁷)SO₂R⁶, C₂-C₁₀alkenyl, C₂-C₁₀alkynyl, C₃-C₈cycloalkyl, C₃-C₈heterocycloalkyl, C₅-C₈cycloalkenyl, aryl or heteroaryl, wherein R⁶, R⁷, and R⁸are the same as defined above;
or a pharmaceutically acceptable salt or solvate thereof.

2. A compound according to claim 1 wherein:
R¹is an unsubstituted or substituted 4 to 8-membered mono-cyclic heteroaryl or a 9-11-membered bicyclic heteroaryl ring;
A is a bond, CH₂;
R²is aryl, C₁-C₁₀alkyl-aryl, heteroaryl, C₁-C₁₀alkyl-heteroaryl, C₃-C₈cycloalkyl-heterocyclyl, hydrocarbyl, (C₂-C₁₀)alkyl-R⁹;
R³, R⁴and R⁵each independently selected from the group consisting of hydrogen, nitro, cyano, halogen, CF₃, —C(O)R⁶, —C(O)OR⁶, —OR⁶, —SR⁶, —S(O)R⁶, —S(O)₂R⁶, —NR⁷R⁸, —CONR⁷R⁸, —N(R⁷)C(O)R⁶, —N(R⁷)C(O)OR⁶, —OC(O)NR⁷R⁸, —N(R⁷)C(O)N⁷R⁸, —P(O)(OR⁶)₂, —SO₂NR⁷R⁸, —N(R⁷)SO₂R⁶, C₁-C₁₀alkyl, C₂-C₁₀alkenyl, C₂-C₁₀alkynyl, C₃-C₈cycloalkyl, C₃-C₈heterocycloalkyl, C₅-C₈cycloalkenyl, aryl, and heteroaryl;
each R⁶is independently selected from the group consisting of hydrogen, C₁-C₁₀alkyl, C₃-C₁₀cycloalkyl, C₃-C₁₀heterocycloalkyl, aryl, and heteroaryl;
R⁷and R⁸are each independently selected from the group consisting of hydrogen, C₁-C₁₀alkyl, C₃-C₁₀cycloalkyl, C₃-C₁₀heterocycloalkyl, aryl, and heteroaryl; or R⁷and R⁸taken together with the nitrogen to which they are attached form a 5- or 6- or 7-membered saturated ring optionally containing one other heteroatom which is oxygen, nitrogen or sulphur;
R⁹is selected from the group consisting of nitro, cyano, halogen, —C(O)R⁶, —C(O)OR⁶, —OR⁶, —SR⁶, —S(O)R⁶—NR⁷R⁸, —CONR⁷R⁸, —N(R⁷)C(O)R⁶, —N(R⁷)C(O)OR⁶, —OC(O)NR⁷R⁸, —N(R⁷)C(O)N⁷R⁸, —P(O)(OR⁶)₂, —SO₂NR⁷R⁸, —N(R⁷)SO₂R⁸;
any carbon or heteroatom of R², R³, R⁴, R⁵, R⁶, R⁷, R⁸, R⁹where possible, is substituted with one or more substituents independently selected from C₁-C₆alkyl, aryl, heteroaryl, halogen, —OR⁶, —NR⁷R⁸, cyano, nitro, —C(O)R⁶, —C(O)OR⁶, —SR⁶, —S(O)R⁶, —S(O)₂R⁶, —CONR⁷R⁸, —N(R⁷)C(O)R⁶, —N(R⁷)C(O)OR⁶, —OC(O)NR⁷R⁸, —N(R⁷)C(O)NR⁷R⁸, —SO₂NR⁷R⁸, —N(R⁷)SO₂R⁶, C₂-C₁₀alkenyl, C₂-C₁₀alkynyl, C₃-C₈cycloalkyl, C₃-C₈heterocycloalkyl, C₅-C₈cycloalkenyl, aryl or heteroaryl, wherein R⁶, R⁷, and R⁸are the same as defined above;
or a pharmaceutically acceptable salt or solvate thereof.

3. A compound according to claim 1 wherein:
R¹is an unsubstituted or substituted 4 to 8-membered mono-cyclic heteroaryl ring;
A is a bond, CH₂
R²is aryl, C₁-C₁₀alkyl-aryl;
R³, R⁴and R⁵each independently selected from the group consisting of hydrogen, nitro, cyano, halogen, CF₃, —C(O)R⁶, —C(O)OR⁶, —OR⁶, —SR⁶, —S(O)R⁶, —S(O)₂R⁶, —NR⁷R⁸, —CONR⁷R⁸, —N(R⁷)C(O)R⁶, —N(R⁷)C(O)OR⁶, —OC(O)NR⁷R⁸, —N(R⁷)C(O)N⁷R⁸, —P(O)(OR⁶)₂, —SO₂NR⁷R⁸, —N(R⁷)SO₂R⁶, C₁-C₁₀alkyl, C₂-C₁₀alkenyl, C₂-C₁₀alkynyl, C₃-C₈cycloalkyl, C₃-C₈heterocycloalkyl, C₅-C₈cycloalkenyl, aryl, and heteroaryl;
each R⁶is independently selected from the group consisting of hydrogen, C₁-C₁₀alkyl, C₃-C₁₀cycloalkyl, C₃-C₁₀heterocycloalkyl, aryl, and heteroaryl;
R⁷and R⁸are each independently selected from the group consisting of hydrogen, C₁-C₁₀alkyl, C₃-C₁₀cycloalkyl, C₃-C₁₀heterocycloalkyl, aryl, and heteroaryl; or R⁷and R⁸taken together with the nitrogen to which they are attached form a 5- or 6- or 7-membered saturated ring optionally containing one other heteroatom which is oxygen, nitrogen or sulphur;
or a pharmaceutically acceptable salt or solvate thereof.

4. A compound according to claim 1 wherein:
R¹is an unsubstituted or substituted 9- to 11-membered bi-cyclic heteroaryl ring;
A is a bond, CH₂
R²is aryl, C₁-C₁₀alkyl-aryl;
R³, R⁴and R⁵each independently selected from the group consisting of hydrogen, nitro, cyano, halogen, CF₃, —C(O)R⁶, —C(O)OR⁶, —OR⁶, —SR⁶, —S(O)R⁶, —S(O)₂R⁶, —NR⁷R⁸, —CONR⁷R⁸, —N(R⁷)C(O)R⁶, —N(R⁷)C(O)OR⁶, —OC(O)NR⁷R⁸, —N(R⁷)C(O)N⁷R⁸, —P(O)(OR⁶)₂, —SO₂NR⁷R⁸, —N(R⁷)SO₂R⁶, C₁-C₁₀alkyl, C₂-C₁₀alkenyl, C₂-C₁₀alkynyl, C₃-C₈cycloalkyl, C₃-C₈heterocycloalkyl, C₅-C₈cycloalkenyl, aryl, and heteroaryl;
each R⁶is independently selected from the group consisting of hydrogen, C₁-C₁₀alkyl, C₃-C₁₀cycloalkyl, C₃-C₁₀heterocycloalkyl, aryl, and heteroaryl;
R⁷and R⁸are each independently selected from the group consisting of hydrogen, C₁-C₁₀alkyl, C₃-C₁₀cycloalkyl, C₃-C₁₀heterocycloalkyl, aryl, and heteroaryl; or R⁷and R⁸taken together with the nitrogen to which they are attached form a 5- or 6- or 7-membered saturated ring optionally containing one other heteroatom which is oxygen, nitrogen or sulphur;
or a pharmaceutically acceptable salt or solvate thereof.

5. A compound according to claim 1 which is:
N-[(3-chlorophenyl)methyl]-3-(3,5-dichloro-2-pyridinyl)-4-oxo-2-thioxo-1,2,3,4-tetrahydro-7-quinazolinecarboxamide;
3-[3,5-bis(methyloxy)-2-pyridinyl]-N-[(3-chlorophenyl)methyl]-4-oxo-2-thioxo-1,2,3,4-tetrahydro-7-quinazolinecarboxamide;
3-[3,5-bis(methyloxy)-2-pyridinyl]-N-[(4-chlorophenyl)methyl]-4-oxo-2-thioxo-1,2,3,4-tetrahydro-7-quinazolinecarboxamide;
3-[4,6-bis(methyloxy)-2-pyrimidinyl]-N-[(3-chlorophenyl)methyl]-4-oxo-2-thioxo-1,2,3,4-tetrahydro-7-quinazolinecarboxamide;
3-[4,6-bis(methyloxy)-2-pyrimidinyl]-N-[(4-chlorophenyl)methyl]-4-oxo-2-thioxo-1,2,3,4-tetrahydro-7-quinazolinecarboxamide;
3-[4,5-bis(methyloxy)-2-pyrimidinyl]-N-[(3-chlorophenyl)methyl]-4-oxo-2-thioxo-1,2,3,4-tetrahydro-7-quinazolinecarboxamide;
3-[4,5-bis(methyloxy)-2-pyrimidinyl]-N-[(4-chlorophenyl)methyl]-4-oxo-2-thioxo-1,2,3,4-tetrahydro-7-quinazolinecarboxamide;
N-[(3-chlorophenyl)methyl]-3-(3-hydroxy-2-pyridinyl)-4-oxo-2-thioxo-1,2,3,4-tetrahydro-7-quinazolinecarboxamide;
N-[(4-chlorophenyl)methyl]-3-(3-hydroxy-2-pyridinyl)-4-oxo-2-thioxo-1,2,3,4-tetrahydro-7-quinazolinecarboxamide;
3-[5,6-bis(methyloxy)-2-pyridinyl]-N-[(3-chlorophenyl)methyl]-4-oxo-2-thioxo-1,2,3,4-tetrahydro-7-quinazolinecarboxamide;
3-[5,6-bis(methyloxy)-2-pyridinyl]-N-[(4-chlorophenyl)methyl]-4-oxo-2-thioxo-1,2,3,4-tetrahydro-7-quinazolinecarboxamide;
N-[(3-chlorophenyl)methyl]-4-oxo-3-(2-pyridinylmethyl)-2-thioxo-1,2,3,4-tetrahydro-7-quinazolinecarboxamide;
N-[(4-chlorophenyl)methyl]-4-oxo-3-(2-pyridinylmethyl)-2-thioxo-1,2,3,4-tetrahydro-7-quinazolinecarboxamide;
N-[(3-chlorophenyl)methyl]-4-oxo-3-(2-pyrimidinyl)-2-thioxo-1,2,3,4-tetrahydro-7-quinazolinecarboxamide;
N-[(4-chlorophenyl)methyl]-4-oxo-3-(2-pyrimidinyl)-2-thioxo-1,2,3,4-tetrahydro-7-quinazolinecarboxamide;
N-[(3-chlorophenyl)methyl]-3-[5-(methyloxy)-2-pyrimidinyl]-4-oxo-2-thioxo-1,2,3,4-tetrahydro-7-quinazolinecarboxamide;
N-[(4-chlorophenyl)methyl]-3-[5-(methyloxy)-2-pyrimidinyl]-4-oxo-2-thioxo-1,2,3,4-tetrahydro-7-quinazolinecarboxamide;
N-[(3-chlorophenyl)methyl]-4-oxo-3-(2-pyridinylamino)-2-thioxo-1,2,3,4-tetrahydro-7-quinazolinecarboxamide trifluoroacetate;
N-[(4-chlorophenyl)methyl]-4-oxo-3-(2-pyridinylamino)-2-thioxo-1,2,3,4-tetrahydro-7-quinazolinecarboxamide trifluoroacetate;
N-[(4-chlorophenyl)methyl]-4-oxo-3-(1,3-thiazol-2-ylmethyl)-2-thioxo-1,2,3,4-tetrahydro-7-quinazolinecarboxamide;
N-[(4-chlorophenyl)methyl]-4-oxo-3-(4-pyrimidinyl)-2-thioxo-1,2,3,4-tetrahydro-7-quinazolinecarboxamide;
N-[(3-chlorophenyl)methyl]-3-(4-hydroxy-2-pyridinyl)-4-oxo-2-thioxo-1,2,3,4-tetrahydro-7-quinazolinecarboxamide;
N-[(4-chlorophenyl)methyl]-3-(4-hydroxy-2-pyridinyl)-4-oxo-2-thioxo-1,2,3,4-tetrahydro-7-quinazolinecarboxamide;
N-[(4-chlorophenyl)methyl]-4-oxo-3-(2-pyridinyl)-2-thioxo-1,2,3,4-tetrahydro-7-quinazolinecarboxamide;
3-[4,6-bis(methyloxy)-2-pyrimidinyl]-N-{[4-(methylsulfonyl)phenyl]methyl}-4-oxo-2-thioxo-1,2,3,4-tetrahydro-7-quinazolinecarboxamide;
N-{[4-(aminosulfonyl)phenyl]methyl}-3-[4,6-bis(methyloxy)-2-pyrimidinyl]-4-oxo-2-thioxo-1,2,3,4-tetrahydro-7-quinazolinecarboxamide;
3-[4,6-bis(methyloxy)-2-pyrimidinyl]-N-({4-[(methylamino)sulfonyl]phenyl}methyl)-4-oxo-2-thioxo-1,2,3,4-tetrahydro-7-quinazolinecarboxamide;
3-[4,6-bis(methyloxy)-2-pyrimidinyl]-N-{[4-(4-morpholinylsulfonyl)phenyl]methyl}-4-oxo-2-thioxo-1,2,3,4-tetrahydro-7-quinazolinecarboxamide;
N-{[4-(acetylamino)phenyl]methyl}-3-[4,6-bis(methyloxy)-2-pyrimidinyl]-4-oxo-2-thioxo-1,2,3,4-tetrahydro-7-quinazolinecarboxamide;
3-{[({3-[4,6-bis(methyloxy)-2-pyrimidinyl]-4-oxo-2-thioxo-1,2,3,4-tetrahydro-7-quinazolinyl}carbonyl)amino]methyl}benzoic acid;
4-{[({3-[4,6-bis(methyloxy)-2-pyrimidinyl]-4-oxo-2-thioxo-1,2,3,4-tetrahydro-7-quinazolinyl}carbonyl)amino]methyl}benzoic acid;
3-[4,6-bis(methyloxy)-2-pyrimidinyl]-4-oxo-N-(2-thienylmethyl)-2-thioxo-1,2,3,4-tetrahydro-7-quinazolinecarboxamide;
3-[4,6-bis(methyloxy)-2-pyrimidinyl]-4-oxo-N-(4-pyridinylmethyl)-2-thioxo-1,2,3,4-tetrahydro-7-quinazolinecarboxamide;
3-[4,6-bis(methyloxy)-2-pyrimidinyl]-N-[3-(4-morpholinyl)propyl]-4-oxo-2-thioxo-1,2,3,4-tetrahydro-7-quinazolinecarboxamide trifluoroacetate;
3-[4,6-bis(methyloxy)-2-pyrimidinyl]-4-oxo-N-(4-piperidinylmethyl)-2-thioxo-1,2,3,4-tetrahydro-7-quinazolinecarboxamide trifluoroacetate;
3-[4,6-bis(methyloxy)-2-pyrimidinyl]-4-oxo-2-thioxo-1,2,3,4-tetrahydro-7-quinazolinecarboxamide;
3-[4,6-bis(methyloxy)-2-pyrimidinyl]-N-{[3-(dimethylamino)phenyl]methyl}-4-oxo-2-thioxo-1,2,3,4-tetrahydro-7-quinazolinecarboxamide;
3-[4,6-bis(methyloxy)-2-pyrimidinyl]-N,N-dimethyl-4-oxo-2-thioxo-1,2,3,4-tetrahydro-7-quinazolinecarboxamide;
3-[4,6-bis(methyloxy)-2-pyrimidinyl]-N-methyl-4-oxo-2-thioxo-1,2,3,4-tetrahydro-7-quinazolinecarboxamide;
3-[4,6-bis(methyloxy)-2-pyrimidinyl]-N-(1-methyl-4-piperidinyl)-4-oxo-2-thioxo-1,2,3,4-tetrahydro-7-quinazolinecarboxamide;
3-[4,5-bis(methyloxy)-2-pyrimidinyl]-4-oxo-N-[3-(phenylcarbonyl)phenyl]-2-thioxo-1,2,3,4-tetrahydro-7-quinazolinecarboxamide;
3-[4,5-bis(methyloxy)-2-pyrimidinyl]-4-oxo-N-[4-(phenyl carbonyl)phenyl]-2-thioxo-1,2,3,4-tetrahydro-7-quinazolinecarboxamide;
3-[4,5-bis(methyloxy)-2-pyrimidinyl]-4-oxo-N-{[4-(phenylcarbonyl)phenyl]methyl}-2-thioxo-1,2,3,4-tetrahydro-7-quinazolinecarboxamide;
3-[4,5-bis(methyloxy)-2-pyrimidinyl]-4-oxo-N-{[3-(phenylcarbonyl)phenyl]methyl}-2-thioxo-1,2,3,4-tetrahydro-7-quinazolinecarboxamide;
3-[4,5-bis(methyloxy)-2-pyrimidinyl]-4-oxo-N-(2-pyridinylmethyl)-2-thioxo-1,2,3,4-tetrahydro-7-quinazolinecarboxamide;
3-[4,5-bis(methyloxy)-2-pyrimidinyl]-4-oxo-N-(3-pyridinylmethyl)-2-thioxo-1,2,3,4-tetrahydro-7-quinazolinecarboxamide;
3-[4,5-bis(methyloxy)-2-pyrimidinyl]-4-oxo-N-(4-pyridinylmethyl)-2-thioxo-1,2,3,4-tetrahydro-7-quinazolinecarboxamide;
N-({3-[4,5-bis(methyloxy)-2-pyrimidinyl]-4-oxo-2-thioxo-1,2,3,4-tetrahydro-7-quinazolinyl}carbonyl)glycine;
3-[4,6-bis(methyloxy)-2-pyrimidinyl]-N-[(3-chlorophenyl)methyl]-6-methyl-4-oxo-2-thioxo-1,2,3,4-tetrahydro-7-quinazolinecarboxamide;
3-[4,6-bis(methyloxy)-2-pyrimidinyl]-N-[(4-chlorophenyl)methyl]-6-methyl-4-oxo-2-thioxo-1,2,3,4-tetrahydro-7-quinazolinecarboxamide;
3-[5,6-bis(methyloxy)-2-pyridinyl]-N-[(3-chlorophenyl)methyl]-6-methyl-4-oxo-2-thioxo-1,2,3,4-tetrahydro-7-quinazolinecarboxamide;
3-[5,6-bis(methyloxy)-2-pyridinyl]-N-[(4-chlorophenyl)methyl]-6-methyl-4-oxo-2-thioxo-1,2,3,4-tetrahydro-7-quinazolinecarboxamide;
3-[5,6-bis(methyloxy)-2-pyridinyl]-N-[(4-chlorophenyl)methyl]-6-(methyloxy)-4-oxo-2-thioxo-1,2,3,4-tetrahydro-7-quinazolinecarboxamide;
3-[5,6-bis(methyloxy)-2-pyridinyl]-N-[(3-chlorophenyl)methyl]-6-(methyloxy)-4-oxo-2-thioxo-1,2,3,4-tetrahydro-7-quinazolinecarboxamide;
3-[4,5-bis(methyloxy)-2-pyrimidinyl]-N-[(3-chlorophenyl)methyl]-6-(methyloxy)-4-oxo-2-thioxo-1,2,3,4-tetrahydro-7-quinazolinecarboxamide;
3-[4,5-bis(methyloxy)-2-pyrimidinyl]-N-[(4-chlorophenyl)methyl]-6-(methyloxy)-4-oxo-2-thioxo-1,2,3,4-tetrahydro-7-quinazolinecarboxamide;
3-[4,5-bis(methyloxy)-2-pyrimidinyl]-6-(methyloxy)-4-oxo-N-(4-pyridinylmethyl)-2-thioxo-1,2,3,4-tetrahydro-7-quinazolinecarboxamide;
3-[4,5-bis(methyloxy)-2-pyrimidinyl]-N-(3-chlorophenyl)-6-(methyloxy)-4-oxo-2-thioxo-1,2,3,4-tetrahydro-7-quinazolinecarboxamide;
3-[5,6-bis(methyloxy)-2-pyridinyl]-6-chloro-N-[(3-chlorophenyl)methyl]-4-oxo-2-thioxo-1,2,3,4-tetrahydro-7-quinazolinecarboxamide;
3-[5,6-bis(methyloxy)-2-pyridinyl]-6-chloro-N-[(4-chlorophenyl)methyl]-4-oxo-2-thioxo-1,2,3,4-tetrahydro-7-quinazolinecarboxamide;
3-[4,5-bis(methyloxy)-2-pyrimidinyl]-6-chloro-N-[(4-chlorophenyl)methyl]-4-oxo-2-thioxo-1,2,3,4-tetrahydro-7-quinazolinecarboxamide;
3-[4,5-bis(methyloxy)-2-pyrimidinyl]-6-chloro-N-[(3-chlorophenyl)methyl]-4-oxo-2-thioxo-1,2,3,4-tetrahydro-7-quinazolinecarboxamide;
3-[5,6-bis(methyloxy)-2-pyridinyl]-5-chloro-N-[(3-chlorophenyl)methyl]-4-oxo-2-thioxo-1,2,3,4-tetrahydro-7-quinazolinecarboxamide;
3-[5,6-bis(methyloxy)-2-pyridinyl]-5-chloro-N-[(4-chlorophenyl)methyl]-4-oxo-2-thioxo-1,2,3,4-tetrahydro-7-quinazolinecarboxamide;
3-[4,5-bis(methyloxy)-2-pyrimidinyl]-5-chloro-N-[(3-chlorophenyl)methyl]-4-oxo-2-thioxo-1,2,3,4-tetrahydro-7-quinazolinecarboxamide;
3-[4,5-bis(methyloxy)-2-pyrimidinyl]-5-chloro-N-[(4-chlorophenyl)methyl]-4-oxo-2-thioxo-1,2,3,4-tetrahydro-7-quinazolinecarboxamide;
3-[5,6-bis(methyloxy)-2-pyridinyl]-N-[(3-chlorophenyl)methyl]-5-methyl-4-oxo-2-thioxo-1,2,3,4-tetrahydro-7-quinazolinecarboxamide;
3-[5,6-bis(methyloxy)-2-pyridinyl]-N-[(4-chlorophenyl)methyl]-5-methyl-4-oxo-2-thioxo-1,2,3,4-tetrahydro-7-quinazolinecarboxamide;
3-[4,5-bis(methyloxy)-2-pyrimidinyl]-N-[(3-chlorophenyl)methyl]-5-methyl-4-oxo-2-thioxo-1,2,3,4-tetrahydro-7-quinazolinecarboxamide;
3-[4,5-bis(methyloxy)-2-pyrimidinyl]-N-[(4-chlorophenyl)methyl]-5-methyl-4-oxo-2-thioxo-1,2,3,4-tetrahydro-7-quinazolinecarboxamide;
3-[5,6-bis(methyloxy)-2-pyridinyl]-N-[(3-chlorophenyl)methyl]-6-fluoro-4-oxo-2-thioxo-1,2,3,4-tetrahydro-7-quinazolinecarboxamide;
3-[5,6-bis(methyloxy)-2-pyridinyl]-N-[(4-chlorophenyl)methyl]-6-fluoro-4-oxo-2-thioxo-1,2,3,4-tetrahydro-7-quinazolinecarboxamide;
3-[4,5-bis(methyloxy)-2-pyrimidinyl]-N-[(3-chlorophenyl)methyl]-6-fluoro-4-oxo-2-thioxo-1,2,3,4-tetrahydro-7-quinazolinecarboxamide;
3-[4,5-bis(methyloxy)-2-pyrimidinyl]-N-[(4-chlorophenyl)methyl]-6-fluoro-4-oxo-2-thioxo-1,2,3,4-tetrahydro-7-quinazolinecarboxamide;
3-[5,6-bis(methyloxy)-2-pyridinyl]-N-[(4-chlorophenyl)methyl]-5-(methyloxy)-4-oxo-2-thioxo-1,2,3,4-tetrahydro-7-quinazolinecarboxamide;
3-[5,6-bis(methyloxy)-2-pyridinyl]-N-[(3-chlorophenyl)methyl]-5-(methyloxy)-4-oxo-2-thioxo-1,2,3,4-tetrahydro-7-quinazolinecarboxamide;
3-[5,6-bis(methyloxy)-2-pyridinyl]-N-[(3-chlorophenyl)methyl]-6-(dimethylamino)-4-oxo-2-thioxo-1,2,3,4-tetrahydro-7-quinazolinecarboxamide;
3-[5,6-bis(methyloxy)-2-pyridinyl]-N-[(4-chlorophenyl)methyl]-6-(dimethylamino)-4-oxo-2-thioxo-1,2,3,4-tetrahydro-7-quinazolinecarboxamide;
3-[2,6-bis(methyloxy)-4-pyrimidinyl]-N-[(4-chlorophenyl)methyl]-4-oxo-2-thioxo-1,2,3,4-tetrahydro-7-quinazolinecarboxamide;
3-[4,6-bis(methyloxy)-5-pyrimidinyl]-N-[(4-chlorophenyl)methyl]-4-oxo-2-thioxo-1,2,3,4-tetrahydro-7-quinazolinecarboxamide;
N-[(4-chlorophenyl)methyl]-3-[6-(methyloxy)-4-pyrimidinyl]-4-oxo-2-thioxo-1,2,3,4-tetrahydro-7-quinazolinecarboxamide;
N-[(4-chlorophenyl)methyl]-4-oxo-3-(2-pyrazinyl)-2-thioxo-1,2,3,4-tetrahydro-7-quinazolinecarboxamide;
N-[(4-chlorophenyl)methyl]-4-oxo-3-(5-pyrimidinyl)-2-thioxo-1,2,3,4-tetrahydro-7-quinazolinecarboxamide;
N-[(4-chlorophenyl)methyl]-4-oxo-3-(3-pyridinyl)-2-thioxo-1,2,3,4-tetrahydro-7-quinazolinecarboxamide;
N-[(4-chlorophenyl)methyl]-4-oxo-3-(4-pyridinyl)-2-thioxo-1,2,3,4-tetrahydro-7-quinazolinecarboxamide;
N-[(4-chlorophenyl)methyl]-3-[6-(methyloxy)-3-pyridinyl]-4-oxo-2-thioxo-1,2,3,4-tetrahydro-7-quinazolinecarboxamide;
6-[7-({[(4-chlorophenyl)methyl]amino}carbonyl)-4-oxo-2-thioxo-1,4-dihydro-3(2H)-quinazolinyl]-3-pyridinecarboxylic acid;
2-[7-({[(4-chlorophenyl)methyl]amino}carbonyl)-4-oxo-2-thioxo-1,4-dihydro-3(2H)-quinazolinyl]-3-pyridinecarboxylic acid;
2-[7-({[(4-chlorophenyl)methyl]amino}carbonyl)-4-oxo-2-thioxo-1,4-dihydro-3(2H)-quinazolinyl]-4-pyridinecarboxylic acid;
6-[7-({[(4-chlorophenyl)methyl]amino}carbonyl)-4-oxo-2-thioxo-1,4-dihydro-3(2H)-quinazolinyl]-2-pyridinecarboxylic acid;
3-[5-(aminocarbonyl)-2-pyridinyl]-N-[(4-chlorophenyl)methyl]-4-oxo-2-thioxo-1,2,3,4-tetrahydro-7-quinazolinecarboxamide;
N-[(4-chlorophenyl)methyl]-4-oxo-3-(3-pyridazinyl)-2-thioxo-1,2,3,4-tetrahydro-7-quinazolinecarboxamide;
3-[6-(acetylamino)-3-pyridinyl]-N-[(4-chlorophenyl)methyl]-4-oxo-2-thioxo-1,2,3,4-tetrahydro-7-quinazolinecarboxamide;
5-[7-({[(4-chlorophenyl)methyl]amino}carbonyl)-4-oxo-2-thioxo-1,4-dihydro-3(2H)-quinazolinyl]-3-pyridinecarboxylic acid;
5-[7-({[(4-chlorophenyl)methyl]amino}carbonyl)-4-oxo-2-thioxo-1,4-dihydro-3(2H)-quinazolinyl]-2-pyridinecarboxylic acid;
N-[(4-chlorophenyl)methyl]-4-oxo-3-(1H-tetrazol-5-yl)-2-thioxo-1,2,3,4-tetrahydro-7-quinazolinecarboxamide;
N-[(4-chlorophenyl)methyl]-4-oxo-3-(1H-tetrazol-5-ylmethyl)-2-thioxo-1,2,3,4-tetrahydro-7-quinazolinecarboxamide;
N-[(4-chlorophenyl)methyl]-3-(3-isoxazolylmethyl)-4-oxo-2-thioxo-1,2,3,4-tetrahydro-7-quinazolinecarboxamide;
N-[(4-chlorophenyl)methyl]-3-[(4-methyl-1,3-thiazol-2-yl)methyl]-4-oxo-2-thioxo-1,2,3,4-tetrahydro-7-quinazolinecarboxamide;
2-[7-({[(4-chlorophenyl)methyl]amino}carbonyl)-4-oxo-2-thioxo-1,4-dihydro-3(2H)-quinazolinyl]-4-methyl-1,3-thiazole-5-carboxylic acid;
3-[6-chloro-5-(methyloxy)-2-pyridinyl]-N-[(4-chlorophenyl)methyl]-4-oxo-2-thioxo-1,2,3,4-tetrahydro-7-quinazolinecarboxamide;
N-[(3-chlorophenyl)methyl]-4-oxo-3-(4-pyrimidinyl)-2-thioxo-1,2,3,4-tetrahydro-7-quinazolinecarboxamide;
N-[(3-chlorophenyl)methyl]-3-(5-chloro-1,3-thiazol-2-yl)-4-oxo-2-thioxo-1,2,3,4-tetrahydro-7-quinazolinecarboxamide;
3-(5-acetyl-4-methyl-1,3-thiazol-2-yl)-N-[(3-chlorophenyl)methyl]-4-oxo-2-thioxo-1,2,3,4-tetrahydro-7-quinazolinecarboxamide;
N-[(3-chlorophenyl)methyl]-3-(5-methyl-1,3,4-thiadiazol-2-yl)-4-oxo-2-thioxo-1,2,3,4-tetrahydro-7-quinazolinecarboxamide;
N-[(3-chlorophenyl)methyl]-3-[6-(methyloxy)-1,3-benzothiazol-2-yl]-4-oxo-2-thioxo-1,2,3,4-tetrahydro-7-quinazolinecarboxamide;
3-[6-chloro-5-(methyloxy)-2-pyridinyl]-N-[(3-chlorophenyl)methyl]-4-oxo-2-thioxo-1,2,3,4-tetrahydro-7-quinazolinecarboxamide;
N-[(3-chlorophenyl)methyl]-3-(5-fluoro-2-pyridinyl)-4-oxo-2-thioxo-1,2,3,4-tetrahydro-7-quinazolinecarboxamide;
N-[(3-chlorophenyl)methyl]-3-(5-cyano-2-pyridinyl)-4-oxo-2-thioxo-1,2,3,4-tetrahydro-7-quinazolinecarboxamide;
N-[(3-chlorophenyl)methyl]-3-(4-methyl-2-pyrimidinyl)-4-oxo-2-thioxo-1,2,3,4-tetrahydro-7-quinazolinecarboxamide;
N-[(3-chlorophenyl)methyl]-3-(5-cyano-2-pyrazinyl)-4-oxo-2-thioxo-1,2,3,4-tetrahydro-7-quinazolinecarboxamide;
N-[(3-chlorophenyl)methyl]-3-[3-cyano-4-(methyloxy)-2-pyridinyl]-4-oxo-2-thioxo-1,2,3,4-tetrahydro-7-quinazolinecarboxamide;
6-[7-({[(3-chlorophenyl)methyl]amino}carbonyl)-4-oxo-2-thioxo-1,4-dihydro-3(2H)-quinazolinyl]-3-pyridinecarboxylic acid;
or a pharmaceutically acceptable salt thereof.

6. A method for treating anemia in a mammal, which method comprises administering an effective amount of a compound of formula (I) or a salt thereof according to claim 1 to a mammal suffering from anemia which can be treated by inhibiting HIF prolyl hydroxylases.

7. A pharmaceutical composition comprising a compound of formula (I) or a salt thereof according to claim 1 and one or more of pharmaceutically acceptable carriers, diluents and excipients.
